(19) United States
(12) Patent Application Publication
(10) Pub. No.: US 2019/0324040 A1
(43) Pub. Date: Oct. 24, 2019

(54) ZIKA VIRUS ANTIBODIES

(71) Applicants: UNIVERSITY OF MIAMI, Miami, FL (US); UNIVERSITY OF SÃO PAULO, State Of São Paulo (BR)

(72) Inventors: David Watkins, Miami, FL (US); Diogo Magnani, Miami, FL (US); Esper Kallas, Miami, FL (US); Sylvia Daunert, Miami, FL (US); Sapna Deo, Miami, FL (US); Emre Dikici, Miami, FL (US)

(21) Appl. No.: 16/345,943

(22) PCT Filed: Oct. 30, 2017
(86) PCT No.: PCT/US17/59129

§ 371 Date: Apr. 29, 2019

Related U.S. Application Data
(60) Provisional application No. 62/414,674, filed on Oct. 29, 2016.

(51) Int. Cl. G01N 33/569; C07K 14/18; C07K 16/10

(52) U.S. Cl. CPC G01N 33/56983; C07K 14/1825; G01N 2333/185; C07K 2317/21; C07K 2317/76; C07K 16/1081

(57) ABSTRACT

Provided herein are Zika virus (ZIKV) binding constructs, e.g., antibodies and antigen-binding fragments thereof, as well as related conjugates, polypeptides, nucleic acids, expression vectors, host cells, kits, and assay systems. Methods detecting ZIKV infection and/or ZIKV exposure and/or ZIKV immunity are provided.

### CROSS-REFERENCE TO RELATED APPLICATIONS

This application claims priority to U.S. Provisional Patent Application No. 62/414,674, filed on Oct. 29, 2016, which is incorporated by reference in its entirety.

### GRANT FUNDING

This invention was made with government support under Grant Nos. 4P01A1094420-05, each of which was awarded by the National Institutes of Health. The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 106,902 byte ACII (Text) file named “51017B_SeqUsting.txt”; created on Oct. 30, 2017.

### BACKGROUND

Zika virus (ZIKV) was isolated from a sentinel Indian rhesus macaque in the Zika forest of Uganda in 1947, although the first manuscript describing the virus was not published until 1952³⁻⁵. The initial descriptions of spontaneous and experimentally-induced human disease followed shortly thereafter^{6,7}. This virus belongs to the genus flavivirus and is related to Dengue virus (DENV), yellow fever virus (YFV), Japanese encephalitis virus (JEV), and west Nile virus (WNV)⁵. Different species of mosquitoes of the Aedes genus are vectors for ZIKV^{8,9}. The potential for the virus to infect the central nervous tissue of mammals was first described in 1971¹⁰. However, ZIKV remained a relatively minor and obscure cause of human disease for most of the second half of the 20^{th} century and was featured in a very limited number of scientific reports. In fact, it was not until 2007 that autochthonous human infection was described outside Africa and continental Asia—in the Federated States of Micronesia¹¹⁻¹³. At that time, the virus caused a mild and self-limited disease characterized by rash, conjunctivitis, and arthralgia and was thus easily confused with DENV or chikungunya virus (CHIKV)^{11,12}. The potential existed for the virus to continue migrating eastward and eventually reach the Americas as a mosquito-borne disease¹². A major change in the epidemiology and clinical manifestations of the virus took place during an outbreak in French Polynesia in late 2013-early 2014 with the first reports of perinatal transmission and association with Guillain-Barre syndrome¹⁴⁻¹⁷. This outbreak was complicated by concurrent outbreaks of DENV and CHIKV transmitted by the same Aedes vector and presenting with similar manifestations and, in some instances, simultaneous infection with ZIKV and DENV in the same patient^{18,19}. By this time, it was also becoming apparent that ZIKV can persist in body fluids such as urine, saliva, and semen beyond the short time (<7 days) that it is present in blood²⁰⁻²³. In fact, the first report of possible sexual transmission of ZIKV was published in 2011: a scientist who had become infected in Senegal in 2008 transmitted the virus to his wife upon his return to Colorado²⁴. The first instances of mosquito-borne transmission in the Americas came from Easter Island, Chile in 2014 and were closely followed by a report of ZIKV infection of eight Brazilian patients in early 2015^{25,26}. Since then, other reports from Brazil have chronicled a rapidly spreading epidemic that, once more, co-exists with transmission of DENV and CHIKV, and is characterized by fever, conjunctivitis, and a maculopapular rash²⁷⁻³¹. The epidemic has spread north with mosquito-borne transmission being reported as far north as Mexico with many nations in the Americas reporting such cases³²⁻³⁴. In early 2016, the first cases were reported on American territory in Puerto Rico³⁵. More ominously, there are reports of microcephaly and ocular damage in aborted fetuses from and infants born to mothers infected with ZIKV with the virus recovered from amniotic fluid, and placental and brain tissue^{2, 36-43}. ZIKV infection has been declared a global public health emergency by the World Health Organization^{44,45}. In the United States, the CDC has issued guidance for the management of the infection in the general population, pregnant women, and possibly affected infants as well as for the prevention of sexual transmission in view of new reports of a possible such occurrence⁴⁶⁻⁵¹. More recently, ZIKV transmission has been described in Miami⁵², suggesting that any region of the United States with Aedes could result in autochthonous spread.

In view of the foregoing, there is a need for rapid diagnostic assays for detecting ZIKV infection in humans. Such assays will allow women to make informed decisions about pregnancy and can assist in preventing sexual transmission of the virus. Rapid diagnostics for both the acute phase and convalescent phase will allow for prevention or control of ZIKV spread. It is particularly important to distinguish ZIKV infection from that of the structurally related dengue virus (DENV) in areas where DENV is endemic and ZIKV is increasing in prevalence. Regions with the highest incidence of ZIKV infection also tend to be resource-limited, so there is an urgent and unmet need for rapid, simple, and cost-effective diagnostics that can specifically identify ZIKV and ZIKV-specific antibody (Ab) responses in body fluids.

### SUMMARY

The present disclosure provides binding constructs, e.g., antibodies or antigen binding fragments thereof, that bind to a ZIKV (e.g., a ZIKV protein) and does not bind to a DENV (e.g., a DENV protein). In exemplary aspects, the binding constructs bind to ZIKV and do not bind to any other flavivirus, including, for example, DENV, YFV, JEV, and WNV. In exemplary aspects, the binding constructs bind to ZIKV and do not bind to the Togaviridae chikungunya virus (CHIKV). In exemplary aspects, the binding construct described herein binds to a ZIKV protein (a protein expressed by ZIKV). In exemplary aspects, the binding construct described herein binds to an epitope within SEQ ID NO: 17. In exemplary aspects, the ZIKV protein is membrane glycoprotein precursor M (SEQ ID NO: 55), or the mature form thereof (membrane glycoprotein M, SEQ ID NO: 56), or envelope protein E (SEQ ID NO: 57). In exemplary aspects, the binding construct comprises a non-human heavy chain constant region and/or a non-human light chain constant region.

The present disclosure provides a polypeptide comprising one or more (e.g., two, three, four, five, or six) of the amino acid sequences of SEQ ID NOs: 1-6, SEQ ID NOs: 21-26, SEQ ID NOs: 29-34, SEQ ID NOs: 37-42, SEQ ID NOs: 45-50 or SEQ ID NOs: 58-63. In exemplary aspects, the polypeptide comprises each of SEQ ID NOs: 1-6 or SEQ ID NOs: 21-26 or SEQ ID NOs: 29-34 or SEQ ID NOs: 37-42 or SEQ ID NOs: 45-50 or SEQ ID NOs: 58-63. In exemplary aspects, the polypeptide comprises SEQ ID NO: 9 and/or SEQ ID NO: 10 or SEQ ID NO: 27 and/or SEQ ID NO: 28 or SEQ ID NO: 35 and/or SEQ ID NO: 36 or SEQ ID NO: 43 and/or SEQ ID NO: 44 or SEQ ID NO: 51 and/or SEQ ID NO: 52 or SEQ ID NO: 64 and/or SEQ ID NO: 65 or SEQ ID NO: 67 and/or SEQ ID NO: 69.

Related nucleic acids encoding the polypeptides or binding constructs of the present disclosure and expression vectors comprising the nucleic acids are also provided herein. Host cells comprising the nucleic acid or the expression vector are further provided herein.

Kits comprising the binding constructs of the present disclosure are provided herein. In exemplary aspects, the kit comprises the binding construct and a solid support. Optionally, the kit comprises a capture molecule which binds to ZIKV.

Assay systems are further provided herein. In exemplary aspects, the assay system comprises a porous matrix comprising at least three zones, Zone A, Zone B, and Zone C, wherein Zone A comprises an antibody or antigen-binding fragment thereof as described herein, wherein the antibody or antigen binding fragment thereof is not bound to a ZIKV, Zone B comprises an antibody or antigen-binding fragment thereof as described herein, wherein the antibody or antigen-binding fragment thereof is bound to a ZIKV, and Zone C comprises a secondary antibody which binds the antibody or antigen-binding fragment thereof of Zone A and Zone B, optionally, wherein the secondary antibody binds to the Fc of the antibody of Zone A and Zone B.

Without being bound to any particular theory, the binding constructs of the present disclosure are particularly useful in diagnostic assays. Thus, the present disclosure provides diagnostic assays wherein one or more of the binding constructs is used. The diagnostic assays of the present disclosure in exemplary aspects detect both ZIKV and serological reactivity against ZIKV. Advantageously, the diagnostic assays provided herein are rapid, easy to use, and simple. Results in exemplary aspects are visualized by the eye in less than 1 hour and need minimal operator expertise. In exemplary aspects, no instrumentation is needed and labor time is reduced. The diagnostic assays of the present disclosure are in exemplary aspects stable and easily transported and have a long shelf life. Accordingly, the diagnostic assays are cost-effective and economical. The total cost of the reagents and materials for an exemplary embodiment of a diagnostic assay for the detection of either ZIKV virus or serological responses to ZIKV is about $2 per test. Advantageously, the diagnostic assay in exemplary aspects is used as a point-of-care (POC) assay.

The present disclosure accordingly provides a method of detecting a ZIKV infection in a subject. In exemplary aspects, the method comprises (i) contacting a sample obtained from the subject with an antibody, antigen-binding fragment, or polypeptide described herein, thereby forming a test mixture, and (ii) assaying the test mixture for a complex comprising ZIKV bound to the antibody, antigen-binding fragment, or polypeptide, wherein, when the complex is present in the test mixture, the subject is determined as having a ZIKV infection.

The present disclosure also provides a method of detecting ZIKV immunity in a subject. In exemplary aspects, the method comprises (i) adding a blood, plasma, or serum sample obtained from the subject to a solid support bound to a capture molecule that binds to ZIKV, (ii) adding a detection antibody comprising an antibody, antigen-binding fragment, or polypeptide described herein, (iii) adding a detection agent which binds to the detection antibody, and (iv) assaying for a signal from the detection agent, wherein, when the signal is detected, the subject is determined as not having ZIKV immunity and, when the signal is not detected, the subject is determined as having ZIKV immunity.

The present disclosure additionally provides a method of detecting a ZIKV infection and ZIKV immunity in a subject. In exemplary aspects, the method comprises adding a sample obtained from a subject to the assay system as described herein, wherein, when the assay system exhibits a single band in Zone C, the subject is determined as having neither a ZIKV infection nor ZIKV immunity, when the assay system exhibits a band in each of Zone A and Zone B, the subject is determined as having both a ZIKV infection and ZIKV immunity, and when the assay system exhibits a band in Zone B and a band is absent in Zone A, the subject is determined as not having a ZIKV infection but having ZIKV immunity.

The present disclosure further provides a method of assessing efficacy of a Zika virus (ZIKV) vaccine in a subject who has received a ZIKV vaccine. In exemplary aspects, the method comprises adding a sample obtained from the subject to the assay system as described herein, wherein, when the assay system exhibits (i) a band in each of Zone A and Zone B or (ii) a band in Zone B and a band is absent in Zone A, the ZIKV vaccine is determined as effective in the subject, and when the assay system exhibits a single band in Zone C, the ZIKV vaccine is determined as ineffective in the subject. In exemplary aspects, the method comprises (i) adding a blood, plasma, or serum sample obtained from the subject to a solid support bound to a capture molecule that binds to ZIKV, (ii) adding a detection antibody comprising an antibody, antigen-binding fragment, or polypeptide described herein, (iii) adding a detection agent which binds to the detection antibody, and (iv) assaying for a signal from the detection agent, wherein, when the signal is detected, the vaccine is determined as ineffective in the subject, and, when the signal is not detected, the vaccine is determined as effective in the subject.

The present disclosure additionally provides a method of treating or preventing a ZIKV infection in a subject. In exemplary aspects, the method comprises administering to the subject a pharmaceutical composition as described herein in an amount effective to treat or prevent the ZIKV injection in the subject. The present disclosure additionally provides a method of inducing an immune response against a ZIKV in a subject. In exemplary aspects, the method comprises administering to the subject a pharmaceutical composition as described herein in an amount effective to induce an immune response against a ZIKV in a subject.

### BRIEF DESCRIPTION OF THE DRAWINGS

FIG. 1 represents a graph of the binding activity of several antibody clones. Human serum 2 is a negative control. Human serum 4 is a position control. CC17 and CC21 bind to ZIKV and do not bind to any of the DENV subtypes.

FIG. 2 represents a detection scheme for determining the presence of ZIKV-specific Abs in the sera of individuals. Sera that do not contain ZIKA specific Abs will not block binding of the CC17 mAb. Only sera from individuals previously infected with ZIKV will block CC17 reactivity.

FIG. 3 represents a serological test of CC17 mAb which accurately predicts ZIKV exposure in 21 of 21 blinded samples. 21 plasma samples from flavivirus-infected individuals in Brazil were analyzed for prior ZIKV-seropositivity using patient plasma to compete with ZIKV binding by CC17 mAb. Patient samples were blinded and included (A) flavivirus-naïve (hu0002), (B) only DENV exposed (138, 01, 02, 04-06, 10-12, 14, 15, 18-20) (C) only ZIKV exposed (hu0004, 03, 09, 21), and (D) DENV and ZIKV exposed (07, 08, 13, 16, 17). Solid lines indicate ZIKV exposure and dashed lines indicate ZIKV naïve.

FIG. 4 represents a scheme for a microtiter-based ZIKV immunoassay using the antibodies of the present disclosure. Concanavalin A is the capture molecule. Detection antibody is an antibody of the present disclosure, e.g., CC17 mAb.

FIG. 5A is a graph of normalized absorbance vs. log of ZIKV particles (copies/mL). This graph demonstrates a dose dependent response of the commercially-available 4G2 antibody as the detection antibody.

FIG. 5B is a graph of normalized absorbance vs. log of ZIKV particles (copies/mL). This graph demonstrates a dose dependent response of the CC21 as the detection antibody.

FIG. 6 is an illustration of a device for the lateral flow assay which detects active ZIKV infection and prior ZIKV exposure.

FIG. 7A is an illustration of the principle behind the lateral flow assay for the detection of active ZIKV infection.

FIG. 7B is an illustration of the principle behind the lateral flow assay for the detection of prior exposure to ZIKV.

FIG. 8 is a graph of absorbance vs. detection antibody concentration (μg/ml). This graph demonstrates a dose dependent response of the CC17 or CC21 as the detection antibody.

FIG. 9 is a graph of viral infectivity neutralization vs. antibody concentration (μg/ml). This graph demonstrates a dose dependent response of the CC17 in neutralizing ZIKV.

FIG. 10 is an illustration of a microtiter-based, ELISA platform diagnostic assay for the direct detection of ZIKV. Shown are the components of the assay and the detection scheme for determining the presence of ZIKV− in an acute ZIKV infection.

FIG. 11A is a graph demonstrating the dose dependent response of CC17 mAb. FIG. 11B is a graph demonstrating the dose dependent response of CC21 mAb. FIG. 11C is a graph demonstrating the dose dependent response of CC4 mAb.

FIG. 12 is a checkerboard assay for the optimization of antibody concentrations.

FIG. 13A is a graph from a time optimization study for ZIKV. FIG. 13B is a graph from a time optimization study for primary antibody. FIG. 13C is a graph from a time optimization study for secondary antibody.

FIG. 14 is a chart demonstrating the extent of mAb binding to ZIKV and the four DENV serotypes was quantified using a virus capture ELISA. The ability of purified mAbs (1 μg/ml) to bind to captured DENV and ZIKV was assessed. Absorbance (Abs 450) values higher than three times the negative control wells were considered binders.

FIG. 15 is a graph demonstrating P1F12 mAb neutralizes ZIKV. P1F12 neutralization curves are presented as the reduction of Vero-cell infectivity measured by flow cytometry.

FIG. 16 demonstrates plasmablast-derived ZIKV-specific mAbs had low SHM levels. FIG. 16A) Number of amino acid mutations from heavy and light chain germline sequences. FIG. 16B) Amino acid alignment of P1F12 to germline genes shows no mutations downstream of cloning primer. Dots “.” indicate sequence identity to the germline gene (shown in each row). Dashes “-” indicate that the Ab does not align to the annotated germline gene sequence on that position. The CDR-H3 sequence is indicated in blue. FIG. 16C) Nucleotide alignment of P1F12 CDR-H3 junction to germline genes. Boxes indicate junctional diversity between V and D (N1), and D and J (N2) gene segments. Framework (FWR) and complementarity-determining regions (CDRs) boundaries are directly annotated on top of the Ab sequence. Antibody regions were determined using IMGT/V-QUEST.

FIG. 17 demonstrates P1F12 mAb binds to whole ZIKV, but not to DENV or recombinant ZIKV E protein. P1F12 binding determined by both Virus Capture ELISA (top panel) and recombinant E protein ELISA (bottom panel) (19 kDa protein without hydrophobic region). Control Absorbances: Whole Virus—Hu0004 (ZIKV+): 2.017, Hu002 (ZIKV−): 0.046. Control Absorbances rE: Whole Virus—Hu0004 (ZIKV+): 2.006, Hu002 (ZIKV−): 0.033.

FIG. 18 demonstrates that inhibition of ZIKV-P1F12 binding discriminates plasma from ZIKV and DENV exposures. A modified virus capture ELISA was conducted to assess the ability of plasma from 46 individuals to block the binding of P1F12 to whole ZIKV. Captured ZIKV was incubated with 1/10 diluted plasma from naïve (US and Brazil) and DENV+, YFV+ or ZIKV+ volunteers prior to addition of purified P1F12. Viral infection was determined by RT-PCR. ZIKV-bound P1F12 was detected using an HRP-conjugated secondary Ab specific for the rhesus IgG1 Fc region of recombinant P1F12. ZIKV+(blue circles), but not ZIKV− (gray circles) plasma inhibited binding of P1F12 mAb to ZIKV.

FIG. 19 demonstrates a P1F12 test assay scheme. 96-well ELISA plates are coated with the P3E11 mAb overnight. The P3E11 is also referenced herein as the CC30 mAb. The next day, plates are washed with PBS-T and blocked with 5% non-fat milk for 1 h at 37° C. The plates are then washed, ZIKV is added to each well, and the plates are incubated at room temperature for 1 h. Plates are washed again, patient plasma or serum is added to wells, and the plates are incubated for 1 h at 37° C. The plates are then washed, the P1F12 ZIKV-specific mAb is added, and incubated for 1 h at 37° C. During this step, if patient plasma was exposed to ZIKV, the patient plasma should block the binding of the P1F12 mAb. Next the plates are washed, a HRP detection mAb is added, and the plates are incubated at 37° C. for 1 h. Lastly, the wells are washed, TMB is used to develop, and the wells are read using a spectrophotometer.

FIG. 20 demonstrates longitudinal assessment of the P1F12 test. The complete and consistent blocking of the P1F12 mAb appears to at approximately day 15 post-onset of symptoms. The patients' ability to block P1F12 binding remains consistent for over a year in both ZIKV infections with and without a history of DENV infection. The dotted line at 0.1 on the y-axis represents the cutoff for ZIKV positivity in the P1F12 test.

FIG. 21 demonstrates True ZIKV-naïve samples. Human plasma and sera were collected from an FDA-approved blood banking site, with no known local ZIKV transmission and tested in the P1F12 test. The dotted line at 0.1 on the y-axis represents the cutoff for ZIKV positivity in the P1F12 test.

FIG. 22 demonstrates immunoglobulin fractionation of plasma samples. Human plasma IgM and IgG were separated using protein G coated agarose beads overnight, the unbound IgM flow through was collected, and the bound IgG fraction was eluted into an equivalent volume. The result of the fractionation was one IgM−/IgG+ fraction and one IgM+/IgG− fraction. The resulting fractions were then run in an ELISA detecting IgM or IgG within each fraction. Within the same ELISA total IgM and total IgG was determined from the unfractionated, original patient plasma.

FIG. 23 demonstrates P1F12 test results from fractionated human plasma. The IgM+/IgG− fraction, IgM−/IgG+ fraction, and whole plasma were all evaluated in a P1F12 test.

### DETAILED DESCRIPTION

### Binding Constructs

### Binding Targets and Epitopes

Provided herein are binding constructs (e.g., an antibody or antigen-binding fragment thereof) which specifically recognize a Zika virus (ZIKV) with minimal or no cross-reactivity to a Dengue virus (DENV). The binding constructs do not bind to any DENV subtype, including, e.g., DENV subtype 1, DENV subtype 2, DENV subtype 3, and DENV subtype 4. In exemplary aspects, the binding constructs bind to ZIKV and do not bind to any other flavivirus. In exemplary aspects, the binding constructs bind to ZIKV even in the presence of other flaviviruses, e.g., DENV, West Nile virus, Yellow fever virus, and the like.

In exemplary embodiments, the binding constructs bind to a ZIKV protein and do not bind to a DENV protein. In exemplary aspects, the binding construct does not bind to a protein of any one of DENV subtype 1, DENV subtype 2, DENV subtype 3, and DENV subtype 4. In exemplary aspects, the binding constructs bind to a protein of a ZIKV comprising the genome of GenBank Accession No. KU926309.1 (SEQ ID NO: 54) or other ZIKV isolates, including, but not limited to the ZIKV comprising a gene or genome of any one of GenBank Accession Nos. KU820897, KU922923, KU820898, KU853012, KU820899, KU744693, KU497555, KU707826, KU527068, KU365777, KU365778, KU365779, KU365780, KU312312, KU321639, AB908162, KU509998, KJ776791, KU681081, KU681082, and EU545988. In exemplary aspects, the ZIKV protein to which the binding constructs bind comprises a fragment of the sequence of SEQ ID NO: 53 or 54. In exemplary aspects, the ZIKV protein to which the binding constructs bind comprises a fragment of SEQ ID NO: 17 or SEQ ID NO: 18. In exemplary aspects, the binding constructs bind to a membrane glycoprotein precursor M (SEQ ID NO: 55), or the mature form thereof (membrane glycoprotein M, SEQ ID NO: 56), or envelope protein E (SEQ ID NO: 57). In exemplary aspects, the binding constructs bind to the ZIKV protein in a sample comprising blood, plasma, serum, urine, or saliva.

In exemplary aspects, the binding constructs bind to a ZIKV molecule which is other than a ZIKV protein. In exemplary aspects, the binding constructs bind to a sugar or lipid from ZIKV or a molecule that is induced by ZIKV infection.

For purposes herein, the phrase “binds to ZIKV”, or a similar phrase, means that the binding construct (e.g., antibody, or antigen-binding fragment) binds to an epitope of a ZIKV protein or ZIKV antigen, and the phrase “do not bind to any DENV subtype” or like phrase, means that the binding construct (e.g., antibody, or antigen-binding fragment) does not bind to an epitope of a DENV protein or DENV antigen. In exemplary aspects, the binding construct has an equilibrium association constant, KA, for ZIKV which is at least 10⁵ mol⁻¹, at least 10⁶ mol⁻¹, at least 10⁷ mol⁻¹, at least 10⁸ mol⁻¹, at least 10⁹ mol⁻¹, or at least 10¹⁰ mol⁻¹. In exemplary aspects, the binding construct has an equilibrium association constant, KA, for DENV which is less than 10³ mol⁻¹. In exemplary aspects, the KD of the binding constructs provided herein for ZIKV is about 1.0×10⁻⁶ or less, about 1.0×10⁻⁷ or less, about 1.0×10⁻⁸ or less, about 1.0×10⁻⁹ or less, about 1.0×10⁻¹⁰ or less. In exemplary aspects, the KD of the binding constructs provided herein for DENV is greater than or about 1.0×10⁻³. In exemplary aspects, the binding construct does not bind to a DENV protein or DENV antigen at a concentration below 10 μg/ml.

By “epitope” as used herein is meant the region of or within a ZIKV antigen which is bound by the binding construct of the present disclosure. In some embodiments, the epitope is a linear epitope. By “linear epitope” as used herein refers to the region of or within the ZIKV protein which is bound by the binding construct and which region is composed of contiguous amino acids of the amino acid sequence of the ZIKV protein. The amino acids of a linear epitope are adjacent to each other in the primary structure of the ZIKV protein. Accordingly, a linear epitope is a fragment or portion of the amino acid sequence of the antigen, i.e., a ZIKV protein. In other exemplary embodiments, the epitope is a conformational or structural epitope. By “conformational epitope” or “structural epitope” is meant an epitope which is composed of amino acids which are located in close proximity to one another when the ZIKV protein is in its properly folded state. Unlike linear epitopes, the amino acids of a conformational or structural epitope need not be adjacent to each other in the primary structure (i.e., amino acid sequence) of the ZIKV protein. A conformational or structural epitope is not necessarily made of contiguous amino acids of the amino acid sequence of the antigen.

In exemplary aspects, the binding constructs of the present disclosure bind to an immunodominant epitope of ZIKV. As used herein, the term “immunodominant epitope” refers to an epitope of a ZIKV antigen on which the immune response focuses through a process called immunodominance. Immunodominant focus determines which epitopes are favored to vary antigenically to escape immune pressure. Immunodominance within hosts is described in Chapter 6 of Frank S A, Immunology and Evolution of Infectious Disease, Princeton University Press, Princeton, N.J., 2002. In exemplary aspects, the binding constructs of the present disclosure bind to an immunodominant epitope which is exclusive to ZIKV, thereby allowing for discrimination between a ZIKV infection and a DENV infection in a subject. In exemplary aspects, the binding constructs of the present disclosure bind to a ZIKV immunodominant epitope, such that sera from ZIKV infected patients block the interaction between the binding construct and the epitope. Suitable assays for testing whether the binding of an antibody is to an immunodominant epitope are known in the art and also provided herein in Example 2.

In exemplary aspects, the binding constructs of the present disclosure bind to an epitope within the amino acid sequence of SEQ ID NO: 17, SEQ ID NO: 18, SEQ ID NO: 53, or SEQ ID NO: 54. In exemplary aspects, the binding constructs of the present disclosure binds to an epitope within the amino acid sequence of a membrane glycoprotein precursor M (SEQ ID NO: 55), or the mature form thereof (membrane glycoprotein M, SEQ ID NO: 56), or envelope protein E (SEQ ID NO: 57). The binding constructs of the present disclosure, however, are not limited to only such binding constructs. Other binding constructs which bind to ZIKV with minimal or no cross-reactivity to a Dengue virus (DENV) are provided herein.

### Affinity and Avidity

The binding constructs provided herein bind to ZIKV in a non-covalent and reversible manner. In exemplary embodiments, the binding strength of the binding construct to ZIKV may be described in terms of its affinity, a measure of the strength of interaction between the binding site of the binding construct and the epitope. In exemplary aspects, the binding constructs provided herein have high-affinity for ZIKV and thus will bind a greater amount of ZIKV in a shorter period of time than low-affinity binding constructs. In exemplary aspects, the binding construct has an equilibrium association constant, KA, which is at least 10⁵ mol⁻¹, at least 10⁶ mol⁻¹, at least 10⁷ mol⁻¹, at least 10⁸ mol⁻¹, at least 10⁹ mol⁻¹, or at least 10¹⁰ mol⁻¹. In exemplary aspects, the binding constructs provided herein exhibit high affinity for ZIKV in human blood, serum, plasma, saliva or urine. In exemplary aspects, the binding construct binds to the ZIKV and does not bind to a DENV in a sample comprising human blood, serum, plasma, saliva or urine. In exemplary aspects, the binding construct binds to the ZIKV even when a substantial amount of DENV or another flavivirus is present in the sample.

In exemplary embodiments, the binding strength of the binding construct to ZIKV may be described in terms of its sensitivity. KD is the equilibrium dissociation constant, a ratio of k_{off}/kₒₙ, between the binding construct and ZIKV. KD and KA are inversely related. The KD value relates to the concentration of the binding construct (the amount of binding construct needed for a particular experiment), and so the lower the KD value (lower concentration), the higher the affinity of the binding construct. In exemplary aspects, the binding strength of the binding construct to ZIKV may be described in terms of KD. In exemplary aspects, the KD of the binding constructs provided herein for ZIKV is about 1.0×10⁻⁶ or less, about 1.0×10⁻⁷ or less, about 1.0×10⁻⁸ or less, about 1.0×10⁻⁹ or less, about 1.0×10⁻¹⁰ or less. In exemplary aspects, the KD of the binding constructs provided herein is micromolar, nanomolar, picomolar or femtomolar. In exemplary aspects, the KD of the binding constructs provided herein is within a range of about 10⁻⁴ to 10⁻⁶ or 10⁻⁷ to 10⁻⁹ or 10⁻¹⁰ to 10⁻¹² or 10⁻¹³ to 10⁻¹⁵.

Avidity gives a measure of the overall strength of an antibody-antigen complex. It is dependent on three major parameters: affinity of the binding construct for the epitope, valency of both the binding construct and ZIKV, and structural arrangement of the parts that interact. The greater a binding construct's valency (number of antigen binding sites), the greater the amount of antigen (ZIKV) it can bind. In exemplary aspects, the binding constructs have a strong avidity for ZIKV. In exemplary aspects, the binding constructs are bivalent. In exemplary aspects, the binding constructs are multivalent.

### Neutralization

In exemplary embodiments, the binding constructs of the present disclosure are neutralizing binding constructs. For example, the binding construct in some aspects is a neutralizing antibody. As used herein, the term “neutralizing binding construct” or “neutralizing antibody” refers to a binding construct or antibody which has the ability to prevent viral entry by binding to regions on the virus involved in the entry process. In exemplary aspects, the binding construct of the present disclosure prevents viral entry at a concentration below about 10 μg per ml. In exemplary aspects, the neutralizing binding construct, e.g., neutralizing antibody, is a broadly neutralizing antibody which recognizes a wide variety of viral glycoproteins on the surface of enveloped viruses or the protein shell of nonenveloped viruses. Neutralizing antibodies and broadly neutralizing antibodies are known in the art. See, e.g., Sankaranarayanan et al., “Broadly Neutralizing Antibodies for therapy of viral Infections” Antibody Tech Journal 6: 1-15 (2016).

### Structure

The binding constructs described herein may be engineered to have one of a multitude of structures. In exemplary aspects, the binding constructs provided herein have a structure of an antibody or antigen-binding fragment thereof. In exemplary aspects, the binding constructs provided herein have a structure based on or derived from an antibody. In exemplary aspects, the binding constructs provided herein have a structure of a synthetic antibody mimic, an engineered protein, or an aptamer, such as those described herein and in McEnaney et al., “Chemically Synthesized Molecules with the Targeting and Effector Functions of Antibodies” J. Am. Chem. Soc., 136 (52): 18034-18043 (2014); Binz and Plückthun, “Engineered proteins as specific binding reagents” Curr Opin Biotechnol. 16(4):459-69 (2005); and Roque et al., “Antibodies and genetically engineered related molecules: production and purification” Biotechnol Prog. 20(3):639-54 (2004).

### Antibodies and Antigen-Binding Fragments

In exemplary embodiments, the binding construct is an antibody. The antibody may be any type of antibody, i.e., immunoglobulin, known in the art. In exemplary embodiments, the antibody is an antibody of class or isotype IgA, IgD, IgE, IgG, or IgM. In exemplary embodiments, the antibody described herein comprises one or more alpha, delta, epsilon, gamma, and/or mu heavy chains. In exemplary embodiments, the antibody described herein comprises zero, one, or more kappa or light chains. In exemplary aspects, the antibody is an IgG antibody and optionally is one of the four human subclasses: IgG1, IgG2, IgG3 and IgG4. Also, the antibody in some embodiments is a monoclonal antibody. In other embodiments, the antibody is a polyclonal antibody.

In some embodiments, the antibody is structurally similar to or derived from a naturally-occurring antibody, e.g., an antibody isolated and/or purified from a mammal, e.g., mouse, rabbit, goat, horse, chicken, hamster, camel, llama, human, and the like. In this regard, the antibody may be considered as a mammalian antibody, e.g., a mouse antibody, rabbit antibody, goat antibody, horse antibody, chicken antibody, hamster antibody, human antibody, and the like. In exemplary aspects, the antibody comprises sequence of only mammalian antibodies. Methods of producing such antibodies are known in the art, some of which are described further herein under the section entitled “Methods of Antibody Production.” In exemplary aspects, the binding construct is a fully human antibody, or does not comprise sequences of non-human antibodies.

In some embodiments, the antibody is a genetically-engineered antibody and does not occur in nature. In exemplary embodiments, the antibody is a single chain antibody, a single domain antibody, a humanized antibody, a chimeric antibody, a CDR-grafted antibody, a humaneered antibody, a bispecific antibody, a trispecific antibody, and the like. Genetic engineering techniques also provide the ability to make fully human antibodies from a non-human source. In some aspects, the genetically-engineered antibody is a single chain antibody (SCA) specific for ZIKV. Methods of making SCAs are known in the art. See, for example, Davis et al., Nature Biotechnology 9: 165-169 (1991).

In some aspects, the antibody is a chimeric antibody. The term “chimeric antibody” is used herein to refer to an antibody-containing constant domains from one species and the variable domains from a second, or more generally, containing stretches of amino acid sequence from at least two species. In particular aspects, the chimeric antibody binds to ZIKV. In exemplary aspects, the antibody of the present disclosure is a chimeric antibody comprising a human antibody variable region and a human antibody constant region, but the variable region is of a human antibody isotype which is different from the human antibody isotype of the constant region. For example, the variable region may be of isotype IgA and the constant region may be of isotype IgG. In exemplary aspects, the antibody of the present disclosure is a chimeric antibody comprising a human antibody variable region and a non-human antibody constant region. In exemplary aspects, the chimeric antibody comprises a human antibody heavy chain variable region, a human antibody light chain variable region and a non-human heavy chain constant region and/or a non-human light chain constant region. In exemplary aspects, the chimeric antibody comprises a human antibody heavy chain variable region, a human antibody light chain variable region and a mouse, goat, rabbit, or non-human primate heavy chain constant region and/or a mouse, goat, rabbit, or non-human primate light chain constant region. In exemplary aspects, the chimeric antibody comprises a heavy chain constant region and a light chain constant region of the Cercopithecidae family of primates. In exemplary aspects, the chimeric antibody comprises a heavy chain constant region and a light chain constant region of a Rhesus monkey antibody. In exemplary aspects, the chimeric antibody comprises a heavy chain constant region comprising the amino acid sequence of SEQ ID NO: 19 and/or a light chain constant region comprising the amino acid sequence of SEQ ID NO: 20.

In some aspects, the antibody is a humanized antibody. The term “humanized” when used in relation to antibodies refers to antibodies having at least CDR regions from a non-human source which are engineered to have a structure and immunological function more similar to true human antibodies than the original source antibodies. For example, humanizing can involve grafting CDR from a non-human antibody, such as a mouse antibody, into a human antibody. Humanizing also can involve select amino acid substitutions to make a non-human sequence look more like a human sequence.

Use of the terms “chimeric or humanized” herein is not meant to be mutually exclusive, and rather, is meant to encompass chimeric antibodies, humanized antibodies, and chimeric antibodies that have been further humanized. Except where context otherwise indicates, statements about (properties of, uses of, testing of, and so on) chimeric antibodies apply to humanized antibodies, and statements about humanized antibodies pertain also to chimeric antibodies. Likewise, except where context dictates, such statements also should be understood to be applicable to antibodies and antigen-binding fragments of such antibodies.

In some aspects, the antibody is a Humaneered™ antibody. Humaneering technology is a proprietary method of KaloBios Pharmaceuticals, Inc. (South San Francisco, Calif.) for converting non-human antibodies into engineered human antibodies. Humaneered™ antibodies have high affinity, and highly similar to human germline antibody sequences. See, e.g., Tomasevic et al., Growth Factors 32: 223-235 (2014).

In exemplary aspects, the antibody is a CDR-grafted antibody specific for ZIKV. Methods of making CDR-grafted antibodies are known in the art. See, for example, Lo, Benny, Antibody Engineering: Methods and Protocols, Volume 248 (2004), which is incorporated by reference in its entirety. In exemplary embodiments, the antibody is engineered to be bispecific, trispecific, or multi-specific, and the antibody comprises two or more distinct antigen-binding regions. In some aspects, the antibody is a bispecific or trispecific antibody specific for ZIKV. Methods of making bispecific or trispecific antibodies are known in the art. See, for example, Marvin and Zhu, Acta Pharmacologica Sinica 26: 649-658 (2005) and U.S. Pat. No. 6,551,592. In exemplary aspects, the binding construct is a bi-specific antigen-binding construct specific for a first epitope of ZIKV and a second epitope of ZIKV. In exemplary embodiments, the antibody is quadroma, heterodimeric bispecific antibody, bispecific antibody fusion, bispecific antibody fragment, a bispecific T-cell engager (BiTE), or a multi-specific antibody. In exemplary embodiments, the antibody is engineered to be bivalent, trivalent, or multivalent. See, e.g., Cuesta et al., “Multivalent antibodies: when design surpasses evolution” Trends in Biotechnology 28, 355-362 (2010); Holliger et al., “Engineered antibody fragments and the rise of single domains” Nat. Biotechnol. 23, 1126-1136 (2005); Chan et al., “Therapeutic antibodies for autoimmunity and inflammation” Nat Rev Immunol 10, 301-316 (2010); Byrne et al., “A tale of two specificities: bispecific antibodies for therapeutic and diagnostic applications” Trends Biotechnol. 31, 621-632 (2013). In exemplary embodiments, the antibody is in monomeric form, while in other embodiments, the antibody is conjugated to one or more antibodies (e.g., each of which recognize the same epitope of the first antibody). Accordingly, in some aspects, the antibody is in dimeric, polymeric, oligomeric, or multimeric form.

In exemplary aspects, the binding construct is an antigen-binding fragment of an antibody or comprises an antigen-binding fragment of an antibody. The antigen-binding fragment (also referred to herein as “antigen-binding portion”) may be an antigen-binding fragment of any of the antibodies described herein. The antigen-binding fragment can be any part of an antibody that has at least one antigen binding site, including, but not limited to, Fab, F(ab′)₂, a monospecific or bispecific Fab₂, a trispecific Fab₃, a monovalent IgG, scFv, dsFv, scFv-Fc, bispecific diabodies, trispecific triabodies, minibodies, or a fragment of IgNAR (e.g., V-NAR), or a fragment of hclgG (e.g., VhH), or bis-scFvs, fragments expressed by a Fab expression library, and the like. In exemplary aspects, the antigen-binding fragment is a domain antibody, VhH domain, V-NAR domain, VH domain, VL domain, or the like. Antibody fragments of the disclosure, however, are not limited to these exemplary types of antibody fragments. In exemplary aspects, the binding construct comprises a Fab fragment. In exemplary aspects, the binding construct comprises two Fab fragments. In exemplary aspects, the binding construct comprises two Fab fragments connected via a linker. In exemplary aspects, the binding construct comprises or is a minibody comprising two Fab fragments. In exemplary aspects, the binding construct comprises or is a minibody comprising two Fab fragments joined via a linker. Minibodies are known in the art. See, e.g., Hu et al., Cancer Res 56: 3055-3061 (1996). In exemplary aspects, the binding construct comprises or is a minibody comprising two Fab fragments joined via a linker, optionally, comprising an alkaline phosphatase domain.

A domain antibody comprises a functional binding unit of an antibody, and can correspond to the variable regions of either the heavy (V_{H}) or light (V_{L}) chains of antibodies. A domain antibody can have a molecular weight of approximately 13 kDa, or approximately one-tenth of a full antibody. Domain antibodies may be derived from full antibodies such as those described herein.

Antibody fragments that contain the antigen-binding, or idiotype, of the antibody molecule may be generated by techniques known in the art. For example, such fragments include, but are not limited to, the F(ab′)₂ fragment which may be produced by pepsin digestion of the antibody molecule; the Fab′ fragments which may be generated by reducing the disulfide bridges of the F(ab′)₂ fragment; and the two Fab′ fragments which may be generated by treating the antibody molecule with papain and a reducing agent.

A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7, 697-704 (1994)).

Recombinant antibody fragments, e.g., scFvs, can also be engineered to assemble into stable multimeric oligomers of high binding avidity and specificity to different target antigens. Such diabodies (dimers), triabodies (trimers) or tetrabodies (tetramers) are well known in the art, see e.g., Kortt et al., Biomol Eng. 2001 18:95-108, (2001) and Todorovska et al., J Immunol Methods. 248:47-66, (2001).

Bispecific antibodies (bscAb) are molecules comprising two single-chain Fv fragments joined via a glycine-serine linker using recombinant methods. The V light-chain (V_{L}) and V heavy-chain (V_{H}) domains of two antibodies of interest in exemplary embodiments are isolated using standard PCR methods. The V_{L} and V_{H} cDNA's obtained from each hybridoma are then joined to form a single-chain fragment in a two-step fusion PCR. Bispecific fusion proteins are prepared in a similar manner. Bispecific single-chain antibodies and bispecific fusion proteins are antibody substances included within the scope of the present disclosure. Exemplary bispecific antibodies are taught in U.S. Patent Application Publication No. 2005-0282233A1 and International Patent Application Publication No. WO 2005/087812, both applications of which are incorporated herein by reference in their entirety.

In exemplary embodiments, the binding construct is a biparatopic antibody, or a biparatopic antigen-binding fragment thereof, having the capability of binding two different non-overlapping epitopes on the same target antigen molecule. By simultaneously binding to the same cell surface targets, biparatopic antibodies and biparatopic antigen-binding fragments thereof may result in enhanced binding avidity, leading to preferential (strong) binding to only cells that express the targets, thus fine-tuning the antibody selectivity. It has been demonstrated that biparatopic antibodies or biparatopic antigen-binding fragments thereof, by simultaneously binding to two different epitopes on the same target molecule, could even potentially acquire new functionality that could not be achieved with the parent antibodies (or antigen-binding fragments) when used alone or in combination. In exemplary aspects, the binding constructs provided herein are biparatopic for ZIKV.

In exemplary embodiments, the antigen-binding fragment is engineered to be bispecific, trispecific, or multi-specific. In exemplary aspects, the antigen-binding fragment comprises two or more distinct antigen-binding regions. In some aspects, the antigen-binding fragment is a bispecific or trispecific antibody specific for ZIKV and at least one other antigen. In exemplary aspects, the binding construct is a bi-specific antigen-binding fragment specific for a first epitope of ZIKV and a second epitope of ZIKV. In exemplary embodiments, the antigen-binding fragment is engineered to be bivalent, trivalent, or multivalent. In exemplary embodiments, the binding construct is a bivalent Fab fragment monospecific for ZIKV. In some embodiments, the antigen-binding fragment is in monomeric form, while in other embodiments, the antigen-binding fragment is conjugated to one or more antigen-binding fragments (e.g., each of which recognize the same epitope of the first antigen-binding fragment). Accordingly, in some aspects, the antigen-binding fragment is dimerized, polymerized, oligomerized, or multimerized. In exemplary aspects, the binding construct is a dimerized Fab fragment.

In exemplary aspects, the binding construct, e.g., antibody or antigen-binding fragment thereof, comprises one or more of the amino acid sequences of SEQ ID NOs: 1-6. In exemplary aspects, the binding construct, e.g., antibody or antigen-binding fragment thereof, comprises two or more (e.g., three, four, five, or all six) of the amino acid sequences of SEQ ID NOs: 1-6. In exemplary aspects, one or more amino acids are present between each of SEQ ID NOs: 1-6. In exemplary aspects, the binding construct comprises one or more of the amino acid sequences of SEQ ID NOs: 11-16. In exemplary aspects, the binding construct, e.g., antibody or antigen-binding fragment thereof, comprises the sequence of SEQ ID NO: 9 or SEQ ID NO: 10 or both SEQ ID NOs: 9 and 10. In exemplary aspects, the binding construct is an antibody of CC17 or P1F12. In exemplary aspects, the binding construct, e.g., antibody or antigen-binding fragment thereof, of the present disclosure further comprises a non-human antibody constant region. In exemplary aspects, the binding construct further comprises a non-human heavy chain constant region and/or a non-human light chain constant region. In exemplary aspects, the binding construct further comprises a mouse, goat, rabbit, or non-human primate heavy chain constant region and/or a mouse, goat, rabbit, or non-human primate light chain constant region. In exemplary aspects, the non-human primate is of the Cercopithecidae family of primates. In exemplary aspects, the binding construct further comprises a heavy chain constant region and a light chain constant region of a Rhesus monkey antibody. In exemplary aspects, the binding construct comprises a heavy chain constant region comprising the amino acid sequence of SEQ ID NO: 19 and/or a light chain constant region comprising the amino acid sequence of SEQ ID NO: 20.

In exemplary aspects, the binding construct, e.g., antibody or antigen-binding fragment thereof, comprises one or more of the amino acid sequences of SEQ ID NOs: 21-26. In exemplary aspects, the binding construct, e.g., antibody or antigen-binding fragment thereof, comprises two or more (e.g., three, four, five, or all six) of the amino acid sequences of SEQ ID NOs: 21-26. In exemplary aspects, one or more amino acids are present between each of SEQ ID NOs: 21-26. In exemplary aspects, the binding construct, e.g., antibody or antigen-binding fragment thereof, comprises the sequence of SEQ ID NO: 27 or SEQ ID NO: 28 or both SEQ ID NOs: 27 and 28. In exemplary aspects, the binding construct is an antibody of CC27 or P1609. In exemplary aspects, the binding construct, e.g., antibody or antigen-binding fragment thereof, of the present disclosure further comprises a non-human antibody constant region. In exemplary aspects, the binding construct further comprises a non-human heavy chain constant region and/or a non-human light chain constant region. In exemplary aspects, the binding construct further comprises a mouse, goat, rabbit, or non-human primate heavy chain constant region and/or a mouse, goat, rabbit, or non-human primate light chain constant region. In exemplary aspects, the non-human primate is of the Cercopithecidae family of primates. In exemplary aspects, the binding construct further comprises a heavy chain constant region and a light chain constant region of Rhesus monkey. In exemplary aspects, the binding construct comprises a heavy chain constant region comprising the amino acid sequence of SEQ ID NO: 19 and/or a light chain constant region comprising the amino acid sequence of SEQ ID NO: 20.

In exemplary aspects, the binding construct, e.g., antibody or antigen-binding fragment thereof, comprises one or more of the amino acid sequences of SEQ ID NOs: 29-34. In exemplary aspects, the binding construct, e.g., antibody or antigen-binding fragment thereof, comprises two or more (e.g., three, four, five, or all six) of the amino acid sequences of SEQ ID NOs: 29-34. In exemplary aspects, one or more amino acids are present between each of SEQ ID NOs: 29-34. In exemplary aspects, the binding construct, e.g., antibody or antigen-binding fragment thereof, comprises the sequence of SEQ ID NO: 35 or SEQ ID NO: 36 or both SEQ ID NOs: 35 and 36. In exemplary aspects, the binding construct is an antibody of CC21or P1H09. In exemplary aspects, the binding construct, e.g., antibody or antigen-binding fragment thereof, of the present disclosure further comprises a non-human antibody constant region. In exemplary aspects, the binding construct further comprises a non-human heavy chain constant region and/or a non-human light chain constant region. In exemplary aspects, the binding construct further comprises a mouse, goat, rabbit, or non-human primate heavy chain constant region and/or a mouse, goat, rabbit, or non-human primate light chain constant region. In exemplary aspects, the non-human primate is of the Cercopithecidae family of primates. In exemplary aspects, the binding construct further comprises a heavy chain constant region and a light chain constant region of Rhesus monkey. In exemplary aspects, the binding construct comprises a heavy chain constant region comprising the amino acid sequence of SEQ ID NO: 19 and/or a light chain constant region comprising the amino acid sequence of SEQ ID NO: 20.

In exemplary aspects, the binding construct, e.g., antibody or antigen-binding fragment thereof, comprises one or more of the amino acid sequences of SEQ ID NOs: 37-42. In exemplary aspects, the binding construct, e.g., antibody or antigen-binding fragment thereof, comprises two or more (e.g., three, four, five, or all six) of the amino acid sequences of SEQ ID NOs: 37-42. In exemplary aspects, one or more amino acids are present between each of SEQ ID NOs: 37-42. In exemplary aspects, the binding construct, e.g., antibody or antigen-binding fragment thereof, comprises the sequence of SEQ ID NO: 43 or SEQ ID NO: 44 or both SEQ ID NOs: 43 and 44. In exemplary aspects, the binding construct is an antibody of CC28 or P4E04. In exemplary aspects, the binding construct, e.g., antibody or antigen-binding fragment thereof, of the present disclosure further comprises a non-human antibody constant region. In exemplary aspects, the binding construct further comprises a non-human heavy chain constant region and/or a non-human light chain constant region. In exemplary aspects, the binding construct further comprises a mouse, goat, rabbit, or non-human primate heavy chain constant region and/or a mouse, goat, rabbit, or non-human primate light chain constant region. In exemplary aspects, the non-human primate is of the Cercopithecidae family of primates. In exemplary aspects, the binding construct further comprises a heavy chain constant region and a light chain constant region of Rhesus monkey. In exemplary aspects, the binding construct comprises a heavy chain constant region comprising the amino acid sequence of SEQ ID NO: 19 and/or a light chain constant region comprising the amino acid sequence of SEQ ID NO: 20.

In exemplary aspects, the binding construct, e.g., antibody or antigen-binding fragment thereof, comprises one or more of the amino acid sequences of SEQ ID NOs: 45-50. In exemplary aspects, the binding construct, e.g., antibody or antigen-binding fragment thereof, comprises two or more (e.g., three, four, five, or all six) of the amino acid sequences of SEQ ID NOs: 45-50. In exemplary aspects, one or more amino acids are present between each of SEQ ID NOs: 45-50. In exemplary aspects, the binding construct, e.g., antibody or antigen-binding fragment thereof, comprises the sequence of SEQ ID NO: 51 or SEQ ID NO: 52 or both SEQ ID NOs: 51 and 52. In exemplary aspects, the binding construct is an antibody of CC29 or P4A02. In exemplary aspects, the binding construct, e.g., antibody or antigen-binding fragment thereof, of the present disclosure further comprises a non-human antibody constant region. In exemplary aspects, the binding construct further comprises a non-human heavy chain constant region and/or a non-human light chain constant region. In exemplary aspects, the binding construct further comprises a mouse, goat, rabbit, or non-human primate heavy chain constant region and/or a mouse, goat, rabbit, or non-human primate light chain constant region. In exemplary aspects, the non-human primate is of the Cercopithecidae family of primates. In exemplary aspects, the binding construct further comprises a heavy chain constant region and a light chain constant region of Rhesus monkey. In exemplary aspects, the binding construct comprises a heavy chain constant region comprising the amino acid sequence of SEQ ID NO: 19 and/or a light chain constant region comprising the amino acid sequence of SEQ ID NO: 20.

In exemplary aspects, the binding construct, e.g., antibody or antigen-binding fragment thereof, comprises one or more of the amino acid sequences of SEQ ID NOs: 58-63. In exemplary aspects, the binding construct, e.g., antibody or antigen-binding fragment thereof, comprises two or more (e.g., three, four, five, or all six) of the amino acid sequences of SEQ ID NOs: 58-63. In exemplary aspects, one or more amino acids are present between each of SEQ ID NOs: 58-63. In exemplary aspects, the binding construct, e.g., antibody or antigen-binding fragment thereof, comprises the sequence of SEQ ID NO: 64 or SEQ ID NO: 65 or both SEQ ID NOs: 64 and 65. In exemplary aspects, the binding construct is an antibody of CC4 or P1604. In exemplary aspects, the binding construct, e.g., antibody or antigen-binding fragment thereof, of the present disclosure further comprises a non-human antibody constant region. In exemplary aspects, the binding construct further comprises a non-human heavy chain constant region and/or a non-human light chain constant region. In exemplary aspects, the binding construct further comprises a mouse, goat, rabbit, or non-human primate heavy chain constant region and/or a mouse, goat, rabbit, or non-human primate light chain constant region. In exemplary aspects, the non-human primate is of the Cercopithecidae family of primates. In exemplary aspects, the binding construct further comprises a heavy chain constant region and a light chain constant region of Rhesus monkey. In exemplary aspects, the binding construct comprises a heavy chain constant region comprising the amino acid sequence of SEQ ID NO: 19 and/or a light chain constant region comprising the amino acid sequence of SEQ ID NO: 20.

In exemplary aspects, the binding construct, e.g., antibody or antigen-binding fragment thereof, comprises one or more of the amino acid sequences of SEQ ID NOs: 106-111. In exemplary aspects, the binding construct, e.g., antibody or antigen-binding fragment thereof, comprises two or more (e.g., three, four, five, or all six) of the amino acid sequences of SEQ ID NOs: 106-111. In exemplary aspects, one or more amino acids are present between each of SEQ ID NOs: 106-111. In exemplary aspects, the binding construct, e.g., antibody or antigen-binding fragment thereof, comprises the sequence of SEQ ID NO: 67 or SEQ ID NO: 69 or both SEQ ID NOs: 67 and 69. In exemplary aspects, the binding construct is an antibody of CC30 or P3E11. In exemplary aspects, the binding construct, e.g., antibody or antigen-binding fragment thereof, of the present disclosure further comprises a non-human antibody constant region. In exemplary aspects, the binding construct further comprises a non-human heavy chain constant region and/or a non-human light chain constant region. In exemplary aspects, the binding construct further comprises a mouse, goat, rabbit, or non-human primate heavy chain constant region and/or a mouse, goat, rabbit, or non-human primate light chain constant region. In exemplary aspects, the non-human primate is of the Cercopithecidae family of primates. In exemplary aspects, the binding construct further comprises a heavy chain constant region and a light chain constant region of Rhesus monkey. In exemplary aspects, the binding construct comprises a heavy chain constant region comprising the amino acid sequence of SEQ ID NO: 19 and/or a light chain constant region comprising the amino acid sequence of SEQ ID NO: 20.

In exemplary aspects, the binding construct, e.g., antibody or antigen-binding fragment thereof, comprises one or more of the amino acid sequences of SEQ ID NOs: 70-105. In exemplary aspects, the binding construct, e.g., antibody or antigen-binding fragment thereof, comprises two of SEQ ID NOs: 70-105. In exemplary aspects, the binding construct, e.g., antibody or antigen-binding fragment thereof, comprises SEQ ID NOs: 70 and 71, SEQ ID NOs: 72 and 73, SEQ ID NOs: 74 and 75, SEQ ID NOs: 76 and 77, SEQ ID NOs: 78 and 79, SEQ ID NOs: 80 and 81, SEQ ID NOs: 82 and 83, SEQ ID NOs: 84 and 85, SEQ ID NOs: 86 and 87, SEQ ID NOs: 88 and 89, SEQ ID NOs: 90 and 91, SEQ ID NOs: 92 and 93, SEQ ID NOs: 94 and 95, SEQ ID NOs: 96 and 97, SEQ ID NOs: 98 and 99, SEQ ID NOs: 100 and 101, SEQ ID NOs: 102 and 103, or SEQ ID NOs: 104 and 105. In exemplary aspects, one or more amino acids are present between each of the above recited SEQ ID NO: In exemplary aspects, the binding construct, e.g., antibody or antigen-binding fragment thereof, of the present disclosure further comprises a non-human antibody constant region. In exemplary aspects, the binding construct further comprises a non-human heavy chain constant region and/or a non-human light chain constant region. In exemplary aspects, the binding construct further comprises a mouse, goat, rabbit, or non-human primate heavy chain constant region and/or a mouse, goat, rabbit, or non-human primate light chain constant region. In exemplary aspects, the non-human primate is of the Cercopithecidae family of primates. In exemplary aspects, the binding construct further comprises a heavy chain constant region and a light chain constant region of Rhesus monkey. In exemplary aspects, the binding construct comprises a heavy chain constant region comprising the amino acid sequence of SEQ ID NO: 19 and/or a light chain constant region comprising the amino acid sequence of SEQ ID NO: 20.

### Methods of Antibody or Antigen-Binding Fragment Production

Suitable methods of making antibodies are known in the art. For instance, standard hybridoma methods are described in, e.g., Harlow and Lane (eds.), Antibodies: A Laboratory Manual, CSH Press (1988), and CA. Janeway et al. (eds.), Immunobiology, 5^{th} Ed., Garland Publishing, New York, N.Y. (2001)). Monoclonal antibodies for use in the methods of the disclosure may be prepared using any technique which provides for the production of antibody molecules by continuous cell lines in culture. These include but are not limited to the hybridoma technique originally described by Koehler and Milstein (Nature 256: 495-497, 1975), the human B-cell hybridoma technique (Kosbor et al., Immunol Today 4:72, 1983; Cote et al., Proc Natl Acad Sci 80: 2026-2030, 1983) and the EBV-hybridoma technique (Cole et al., Monoclonal Antibodies and Cancer Therapy, Alan R Liss Inc, New York N.Y., pp 77-96, (1985). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1). Antibodies may also be produced by inducing in vivo production in the lymphocyte population or by screening recombinant immunoglobulin libraries or panels of highly specific binding reagents as disclosed in Orlandi et al (Proc Natl Acad Sci 86: 3833-3837; 1989), and Winter G and Milstein C (Nature 349: 293-299, 1991). If the full sequence of the antibody or antigen-binding fragment is known, then methods of producing recombinant proteins may be employed. See, e.g., “Protein production and purification” Nat Methods 5(2): 135-146 (2008).

Phage display also can be used to generate the antibody of the present disclosures. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3^{rd} Edition, Cold Spring Harbor Laboratory Press, New York (2001)). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150). Related methods also are described in U.S. Pat. Nos. 5,403,484; 5,571,698; 5,837,500; 5,702,892. The techniques described in U.S. Pat. Nos. 5,780,279; 5,821,047; 5,824,520; 5,855,885; 5,858,657; 5,871,907; 5,969,108; 6,057,098; and 6,225,447.

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods for generating humanized antibodies are well known in the art and are described in detail in, for example, Janeway et al., supra, U.S. Pat. Nos. 5,225,539, 5,585,089 and 5,693,761, European Patent No. 0239400 BI, and United Kingdom Patent No. 2188638. Humanized antibodies can also be generated using the antibody resurfacing technology described in U.S. Pat. No. 5,639,641 and Pedersen et al., J. Mol. Biol, 235, 959-973 (1994). A preferred chimeric or humanized antibody has a human constant region, while the variable region, or at least a CDR, of the antibody is derived from a non-human species. Methods for humanizing non-human antibodies are well known in the art. (See U.S. Pat. Nos. 5,585,089, and 5,693,762.)

Techniques developed for the production of “chimeric antibodies”, the splicing of mouse antibody genes to human antibody genes to obtain a molecule with appropriate antigen specificity and biological activity, can be used (Morrison et al., Proc Natl Acad Sci 81: 6851-6855 (1984); Neuberger et al., Nature 312: 604-608 (1984); Takeda et al., Nature 314: 452-454 (1985)). Alternatively, techniques described for the production of single chain antibodies (U.S. Pat. No. 4,946,778) can be adapted to produce ZIKV-specific single chain antibodies.

Likewise, using techniques known in the art to isolate CDRs, compositions comprising CDRs are generated. Compositions comprising one, two, and/or three CDRs of a heavy chain variable region or a light chain variable region of a monoclonal antibody can be generated. The CDRs of exemplary antibodies are provided herein as SEQ ID NOs: 1-6, 21-26, 29-34, 37-42, 45-50, and 58-63. Techniques for cloning and expressing nucleotide and polypeptide sequences are well-established in the art (see e.g. Sambrook et al., Molecular Cloning: A Laboratory Manual, 2^{nd} Edition, Cold Spring Harbor, N.Y. (1989)). The amplified CDR sequences are ligated into an appropriate expression vector. The vector comprising one, two, three, four, five and/or six cloned CDRs optionally contains additional polypeptide encoding regions linked to the CDR.

Chemically constructed bispecific antibodies may be prepared by chemically cross-linking heterologous Fab or F(ab′)₂ fragments by means of chemicals such as heterobifunctional reagent succinimidyl-3-(2-pyridyldithiol)-propionate (SPDP, Pierce Chemicals, Rockford, Ill.). The Fab and F(ab′)₂ fragments can be obtained from intact antibody by digesting it with papain or pepsin, respectively (Karpovsky et al., J. Exp. Med. 160:1686-701 (1984); Titus et al., J. Immunol., 138:4018-22 (1987)).

Methods of testing antibodies for the ability to bind to the epitope of the ZIKV regardless of how the antibodies are produced are known in the art and include any antibody-antigen binding assay, such as, for example, radioimmunoassay (RIA), ELISA, Western blot, immunoprecipitation, surface plasmon resonance, and competitive inhibition assays (see, e.g., Janeway et al., infra, and U.S. Patent Application Publication No. 2002/0197266).

### Polypeptides

A polypeptide comprising one or more (e.g., two, three, four, five, six) of the amino acid sequences of SEQ ID NOs: 1-6 is further provided herein. In exemplary aspects, the amino acid sequence of the polypeptide comprises additional sequences of, e.g., intervening amino acids or amino acid sequences. In exemplary aspects, the polypeptide further comprises one or more of the amino acid sequences of SEQ ID NOs: 11-16. In exemplary aspects, the polypeptide comprises an amino acid sequence of SEQ ID NOs: 1-3 and optionally, SEQ ID NOs: 11-13. In exemplary aspects, the polypeptide comprises the amino acid sequence of SEQ ID NO: 9. In exemplary aspects, the polypeptide comprises an amino acid sequence of SEQ ID NOs: 4-6 and optionally SEQ ID NOs: 14-16. In exemplary aspects, the polypeptide comprises the amino acid sequence of SEQ ID NO: 10. In exemplary aspects, the polypeptide comprises all of SEQ ID NOs: 1-6 and 11-16. In exemplary aspects, the polypeptide comprises both SEQ ID NOs: 9 and 10. In exemplary aspects, the polypeptide further comprises the amino acid sequence of SEQ ID NO: 19 and/or SEQ ID NO: 20.

A polypeptide comprising one or more (e.g., two, three, four, five, six) of the amino acid sequences of SEQ ID NOs: 21-26 is further provided herein. In exemplary aspects, the amino acid sequence of the polypeptide comprises additional sequences of, e.g., intervening amino acids or amino acid sequences. In exemplary aspects, the polypeptide comprises the amino acid sequences of SEQ ID NO: 21-23. In exemplary aspects, the polypeptide comprises the amino acid sequence of SEQ ID NO: 27. In exemplary aspects, the polypeptide comprises the amino acid sequences of SEQ ID NO: 24-26. In exemplary aspects, the polypeptide comprises the amino acid sequence of SEQ ID NO: 28. In exemplary aspects, the polypeptide comprises all of SEQ ID NOs: 21-26. In exemplary aspects, the polypeptide comprises both SEQ ID NOs: 27 and 28. In exemplary aspects, the polypeptide further comprises the amino acid sequence of SEQ ID NO: 19 and/or SEQ ID NO: 20.

A polypeptide comprising one or more (e.g., two, three, four, five, six) of the amino acid sequences of SEQ ID NOs: 29-34 is further provided herein. In exemplary aspects, the amino acid sequence of the polypeptide comprises additional sequences of, e.g., intervening amino acids or amino acid sequences. In exemplary aspects, the polypeptide comprises the amino acid sequences of SEQ ID NO: 29-31. In exemplary aspects, the polypeptide comprises the amino acid sequence of SEQ ID NO: 35. In exemplary aspects, the polypeptide comprises the amino acid sequences of SEQ ID NO: 32-34. In exemplary aspects, the polypeptide comprises the amino acid sequence of SEQ ID NO: 36. In exemplary aspects, the polypeptide comprises all of SEQ ID NOs: 29-34. In exemplary aspects, the polypeptide comprises both SEQ ID NOs: 35 and 36. In exemplary aspects, the polypeptide further comprises the amino acid sequence of SEQ ID NO: 19 and/or SEQ ID NO: 20.

A polypeptide comprising one or more of the amino acid sequences of SEQ ID NOs: 37-42 is further provided herein. In exemplary aspects, the amino acid sequence of the polypeptide comprises additional sequences of, e.g., intervening amino acids or amino acid sequences. In exemplary aspects, the polypeptide comprises the amino acid sequences of SEQ ID NO: 37-39. In exemplary aspects, the polypeptide comprises the amino acid sequence of SEQ ID NO: 43. In exemplary aspects, the polypeptide comprises the amino acid sequences of SEQ ID NO: 40-42. In exemplary aspects, the polypeptide comprises the amino acid sequence of SEQ ID NO: 44. In exemplary aspects, the polypeptide comprises all of SEQ ID NOs: 37-42. In exemplary aspects, the polypeptide comprises both SEQ ID NOs: 43 and 44. In exemplary aspects, the polypeptide further comprises the amino acid sequence of SEQ ID NO: 19 and/or SEQ ID NO: 20.

A polypeptide comprising one or more of the amino acid sequences of SEQ ID NOs: 45-50 is further provided herein. In exemplary aspects, the amino acid sequence of the polypeptide comprises additional sequences of, e.g., intervening amino acids or amino acid sequences. In exemplary aspects, the polypeptide comprises the amino acid sequences of SEQ ID NO: 45-47. In exemplary aspects, the polypeptide comprises the amino acid sequence of SEQ ID NO: 51. In exemplary aspects, the polypeptide comprises the amino acid sequences of SEQ ID NO: 48-50. In exemplary aspects, the polypeptide comprises the amino acid sequence of SEQ ID NO: 52. In exemplary aspects, the polypeptide comprises all of SEQ ID NOs: 45-50. In exemplary aspects, the polypeptide comprises both SEQ ID NOs: 51 and 52. In exemplary aspects, the polypeptide further comprises the amino acid sequence of SEQ ID NO: 19 and/or SEQ ID NO: 20.

A polypeptide comprising one or more of the amino acid sequences of SEQ ID NOs: 58-63 is further provided herein. In exemplary aspects, the amino acid sequence of the polypeptide comprises additional sequences of, e.g., intervening amino acids or amino acid sequences. In exemplary aspects, the polypeptide comprises the amino acid sequences of SEQ ID NO: 58-63. In exemplary aspects, the polypeptide comprises the amino acid sequence of SEQ ID NO: 64. In exemplary aspects, the polypeptide comprises the amino acid sequences of SEQ ID NO: 58-60. In exemplary aspects, the polypeptide comprises the amino acid sequence of SEQ ID NO: 65. In exemplary aspects, the polypeptide comprises all of SEQ ID NOs: 61-63. In exemplary aspects, the polypeptide comprises both SEQ ID NOs: 64 and 65. In exemplary aspects, the polypeptide further comprises the amino acid sequence of SEQ ID NO: 19 and/or SEQ ID NO: 20.

A polypeptide comprising one or more of the amino acid sequences of SEQ ID NOs: 106-111 is further provided herein. In exemplary aspects, the amino acid sequence of the polypeptide comprises additional sequences of, e.g., intervening amino acids or amino acid sequences. In exemplary aspects, the polypeptide comprises the amino acid sequences of SEQ ID NO: 106-111. In exemplary aspects, the polypeptide comprises the amino acid sequence of SEQ ID NO: 67. In exemplary aspects, the polypeptide comprises the amino acid sequences of SEQ ID NO: 106-108. In exemplary aspects, the polypeptide comprises the amino acid sequence of SEQ ID NO: 69. In exemplary aspects, the polypeptide comprises all of SEQ ID NOs: 109-111. In exemplary aspects, the polypeptide comprises both SEQ ID NOs: 67 and 69. In exemplary aspects, the polypeptide further comprises the amino acid sequence of SEQ ID NO: 19 and/or SEQ ID NO: 20.

Further provided herein is a polypeptide comprising one or more of the amino acid sequences of SEQ ID NOs: 70-105. In exemplary aspects, the polypeptide comprises SEQ ID NOs: 70 and 71, SEQ ID NOs: 72 and 73, SEQ ID NOs: 74 and 75, SEQ ID NOs: 76 and 77, SEQ ID NOs: 78 and 79, SEQ ID NOs: 80 and 81, SEQ ID NOs: 82 and 83, SEQ ID NOs: 84 and 85, SEQ ID NOs: 86 and 87, SEQ ID NOs: 88 and 89, SEQ ID NOs: 90 and 91, SEQ ID NOs: 92 and 93, SEQ ID NOs: 94 and 95, SEQ ID NOs: 96 and 97, SEQ ID NOs: 98 and 99, SEQ ID NOs: 100 and 101, SEQ ID NOs: 102 and 103, or SEQ ID NOs: 104 and 105. In exemplary aspects, the polypeptide further comprises the amino acid sequence of SEQ ID NO: 19 and/or SEQ ID NO: 20.

In exemplary aspects, the polypeptide of the present disclosure binds to ZIKV and not to DENV or any other flavivirus. In exemplary aspects, the polypeptide binds to only ZIKV even in the presence of DENV, optionally, even in the presence of other flavivirus proteins, e.g., proteins of WNV, JEV, YFV.

### Modified Binding Constructs and Conjugates

The binding constructs described herein can be modified, for instance, by glycosylation, amidation, carboxylation, or phosphorylation, or by the creation of acid addition salts, amides, esters, in particular C-terminal esters, and N-acyl derivatives. Such modified binding constructs disclosed herein may have additional activities, enhanced or reduced biological activity, or other characteristics, such as increased or decreased half-life, as compared to the non-derivatized molecules.

In exemplary embodiments, the binding constructs of the present disclosure are attached, linked, joined, or conjugated to a second moiety (e.g., a heterologous moiety) and the resulting product is a conjugate. Accordingly, provided herein are conjugates comprising the binding constructs described herein (covalently or non-covalently) linked to a heterologous moiety. As used herein, the term “heterologous moiety” refers to any molecule (chemical or biochemical, naturally-occurring or non-coded) which is different from the binding constructs of the invention. Exemplary heterologous moieties include, but are not limited to, a polymer, a carbohydrate, a lipid, a nucleic acid, an oligonucleotide, a DNA or RNA, an amino acid, peptide, polypeptide, protein, therapeutic agent, (e.g., a cytotoxic agent, cytokine), an element or metal, a virus, a diagnostic agent or a detecting agent.

### Conjugates: Fc Fusions

For substituents such as an Fc region of human IgG, the fusion can be fused directly to a binding construct of the invention or fused through an intervening sequence. For example, a human IgG hinge, CH2 and CH3 region may be fused at either the N-terminus or C-terminus of a binding construct to attach the Fc region. The resulting Fc-fusion construct enables purification via a Protein A affinity column (Pierce, Rockford, Ill.). Peptide and proteins fused to an Fc region can exhibit a substantially greater half-life in vivo than the unfused counterpart. A fusion to an Fc region allows for dimerization/multimerization of the fusion polypeptide. The Fc region may be a naturally occurring Fc region, or may be modified for superior characteristics, e.g., therapeutic or diagnostic qualities, circulation time, reduced aggregation. As noted above, in some embodiments, the binding constructs are conjugated, e.g., fused to an immunoglobulin or portion thereof (e.g., variable region, CDR, or Fc region). Known types of immunoglobulins (Ig) include IgG, IgA, IgE, IgD or IgM. The Fc region is a C-terminal region of an Ig heavy chain, which is responsible for binding to Fc receptors that carry out activities such as recycling (which results in prolonged half-life), antibody dependent cell-mediated cytotoxicity (ADCC), and complement dependent cytotoxicity (CDC).

For example, according to some definitions the human IgG heavy chain Fc region stretches from Cys226 to the C-terminus of the heavy chain. The “hinge region” generally extends from Glu216 to Pro230 of human IgG1 (hinge regions of other IgG isotypes may be aligned with the IgG1 sequence by aligning the cysteines involved in cysteine bonding). The Fc region of an IgG includes two constant domains, CH2 and CH3. The CH2 domain of a human IgG Fc region usually extends from amino acids 231 to amino acid 341. The CH3 domain of a human IgG Fc region usually extends from amino acids 342 to 447. References made to amino acid numbering of immunoglobulins or immunoglobulin fragments, or regions, are all based on Kabat et al. 1991, Sequences of Proteins of Immunological Interest, U.S. Department of Public Health, Bethesda, Md. In related embodiments, the Fc region may comprise one or more native or modified constant regions from an immunoglobulin heavy chain, other than CH1, for example, the CH2 and CH3 regions of IgG and IgA, or the CH3 and CH4 regions of IgE.

Suitable heterologous moieties include portions of immunoglobulin sequence that include the FcRn binding site. FcRn, a salvage receptor, is responsible for recycling immunoglobulins and returning them to circulation in blood. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The major contact sites include amino acid residues 248, 250-257, 272, 285, 288, 290-291, 308-311, and 314 of the CH2 domain and amino acid residues 385-387, 428, and 433-436 of the CH3 domain.

Amino acid modifications may be made to the Fc region of an immunoglobulin. Such variant Fc regions comprise at least one amino acid modification in the CH3 domain of the Fc region (residues 342-447) and/or at least one amino acid modification in the CH2 domain of the Fc region (residues 231-341). Mutations believed to impart an increased affinity for FcRn include T256A, T307A, E380A, and N434A (Shields et al. 2001, J. Biol. Chem. 276:6591). Other mutations may reduce binding of the Fc region to FcγRI, FcγRIIA, FcγRIIB, and/or FcγRIIIA without significantly reducing affinity for FcRn. For example, substitution of the Asn at position 297 of the Fc region with Ala or another amino acid removes a highly conserved N-glycosylation site and may result in reduced immunogenicity with concomitant prolonged half-life of the Fc region, as well as reduced binding to FcγRs (Routledge et al. 1995, Transplantation 60:847; Friend et al. 1999, Transplantation 68:1632; Shields et al. 1995, J. Biol. Chem. 276:6591). Amino acid modifications at positions 233-236 of IgG1 have been made that reduce binding to FcγRs (Ward and Ghetie 1995, Therapeutic Immunology 2:77 and Armour et al. 1999, Eur. J. Immunol. 29:2613). Some exemplary amino acid substitutions are described in U.S. Pat. Nos. 7,355,008 and 7,381,408, each incorporated by reference herein in its entirety.

### Heterologous Moieties: Polymers, Carbohydrates, Lipids, Elements, Metals, Viruses, Therapeutic Agents

In exemplary embodiments, the heterologous moiety is a polymer. The polymer may be branched or unbranched. The polymer may be of any molecular weight. The polymer in some embodiments has an average molecular weight of between about 2 kDa to about 100 kDa (the term “about” indicating that in preparations of a water soluble polymer, some molecules will weigh more, some less, than the stated molecular weight). In some embodiments, the polymer is modified to have a single reactive group, such as an active ester for acylation or an aldehyde for alkylation, so that the degree of polymerization may be controlled. The polymer in some embodiments is water soluble so that the protein to which it is attached does not precipitate in an aqueous environment, such as a physiological environment. In some embodiments, when, for example, the composition is used for therapeutic use, the polymer is pharmaceutically acceptable. Additionally, in some aspects, the polymer is a mixture of polymers, e.g., a co-polymer, a block co-polymer. In exemplary aspects, the heterologous moiety is a polymer, optionally, polystyrene or nitrocellulose.

In some embodiments, the heterologous moiety is a carbohydrate. In some embodiments, the carbohydrate is a monosaccharide (e.g., glucose, galactose, fructose), a disaccharide (e.g., sucrose, lactose, maltose), an oligosaccharide (e.g., raffinose, stachyose), a polysaccharide (a starch, amylase, amylopectin, cellulose, chitin, callose, laminarin, xylan, mannan, fucoidan, galactomannan.

In some embodiments, the heterologous moiety is a lipid. The lipid, in some embodiments, is a fatty acid, eicosanoid, prostaglandin, leukotriene, thromboxane, N-acyl ethanolamine), glycerolipid (e.g., mono-, di-, tri-substituted glycerols), glycerophospholipid (e.g., phosphatidylcholine, phosphatidylinositol, phosphatidylethanolamine, phosphatidylserine), sphingolipid (e.g., sphingosine, ceramide), sterol lipid (e.g., steroid, cholesterol), prenol lipid, saccharolipid, or a polyketide, oil, wax, cholesterol, sterol, fat-soluble vitamin, monoglyceride, diglyceride, triglyceride, a phospholipid.

In exemplary aspects, the heterologous moiety is an element, such as a gold particle or other metal. In exemplary aspects, the heterologous moiety is a virus. In exemplary aspects, the virus is ZIKV. In some embodiments, the heterologous moiety is a therapeutic agent. The therapeutic agent may be any of those known in the art.

### Conjugates: Detecting Agents

In exemplary embodiments, the binding construct is conjugated to a detecting agent. In exemplary embodiments, the detecting agent is capable of emitting a detectable (measurable) signal based on enzymatic activity, radioactivity, chromogenic activity, and/or binding activity. In exemplary embodiments, the signal is radioactive, chromogenic, colorimetric, fluorometric, chemiluminescent, enhanced chemiluminescent, direct fluorescent, time-resolved fluorescent, direct chemiluminescent, phosphorescent, enzymatic, or based on binding of a micro- or nanoparticle, streptavidin/avidin-biotin and protein A. In exemplary embodiments, the detecting agent comprises an enzyme, a radioactive isotope, a DNA reporter, a chromogenic or fluorogenic reporter, or an electrochemiluminescent tag. In exemplary aspects, the enzyme is horseradish peroxidase (HRP), alkaline phosphatase (AP), glucose oxidase, or beta-galactosidase. In exemplary aspects, the enzyme when exposed to certain reagents causes chemiluminescence or light production. In exemplary aspects, the radioisotope is I¹²⁵. In exemplary aspects, the DNA reporter is a DNA probe. In exemplary aspects, the fluorogenic reporter is phycoerythrin (PE), e.g., B-PE, R-PE, or allophycocyanin (APC). In exemplary aspects, the detecting agent is a a fluorophore, chromophore, radioisotope, enzymatic label, or biotin.

The binding constructs in exemplary aspects is linked to a detecting agent (e.g., a detectable label or a reporter group), including, but not limited to a radiolabel, a fluorescent label, an enzyme (e.g., that catalyzes a calorimetric or fluorometric reaction), a substrate, a solid matrix, or a carrier (e.g., biotin or avidin). In exemplary aspects, the fluorescent label comprises a rhodamine dye, fluorescein dye and/or a cyanine dye. In exemplary embodiments, the fluorescent label comprises a set of dyes, e.g., a rhodamine dye, TAMRA, and a fluorescein dye, FAM. In another embodiment, the fluorescent label comprises of a set of fluorescent dyes formed by selecting two or more dyes from the group consisting of Oregon Green 488, Flitorescein-EX, fluorescein isothiocyanate, Rhodamine Red-X, Lissamine rhodamine B, Calcein, Fluorescein, Rhodamine, one or more BODIPY dyes, Texas Red, Oregon Green 514, and one or more Alexa Fhiors. Representative BODIPY dyes include BODIPY FL, BODIPY R6G, BOD1PY™ R, BOD1PY 581/591, BODIPY TR, BODIPY 630/650 and BODIPY 650/665. Representative Alexa Fluors include Alexa Fluor 350, 405, 430, 488, 500, 514, 532, 546, 555, 568, 594, 610, 633, 635, 647, 660, 680, 700, 750 and 790. In exemplary aspects, the fluorescent label comprises one or more of Oregon Green 488, fluorescein-EX, FITC, Rhodamine Red-X, Lissamine rhodamine B, calcein, fluorescein, rhodamine, BODIPYS, and Texas Red, e.g. which are disclosed in Molecular Probes Handbook, 11th Edition (2010). In exemplary aspects, the detectable label is selected from radioisotopes, chromophores, fluorophores, fluorochromes, enzymes (e.g., horseradish peroxidase), linker molecules or other moieties or compounds which either emit a detectable signal (e.g., radioactivity, fluorescence, color) or emit a detectable signal after exposure of the label to its substrate. A variety of detectable label/substrate pairs (e.g., horseradish peroxidase/diaminobenzidine, biotin/streptavidin, luciferase/luciferin), methods for labeling antibodies, and methods for using labeled secondary antibodies to detect an antigen are well known in the art. See, e.g., Harlow and Lane, eds. (Using Antibodies: A Laboratory Manual (1999) Cold Spring Harbor Laboratory Press, Cold Spring Harbor, N.Y.).

### Conjugates: Dimers & Multimers

In some embodiments, the binding construct is provided as a dimer or a multimer in which more than one binding construct of the invention are linked together. The dimer in some aspects is a homodimer comprising two binding constructs of the same type (e.g., same structure) linked together. In alternative aspects, the dimer is a heterodimer comprising two binding constructs of the invention, wherein the two binding constructs are structurally distinct from each other. The multimer in some aspects is a homomultimer comprising more than one binding construct of the invention and each binding construct is of the same type (e.g., same structure). In alternative aspects, the multimer is a heteromultimer comprising more than one binding construct of the invention and wherein at least two binding constructs of the heteromultimer are structurally distinct from the other. Two or more of the binding constructs can be linked together using standard linking agents and procedures known to those skilled in the art. In certain embodiments, the linker connecting the two (or more) binding constructs is a linker known in the art. In some embodiments, the linker is a disulfide bond. For example, each monomer of the dimer may comprise a sulfhydryl and the sulfur atom of each participates in the formation of the disulfide bond.

### Nucleic Acids

Further provided herein are nucleic acids comprising a nucleotide sequence encoding any of the binding constructs (e.g., antibodies, antigen-binding fragments) or polypeptides or conjugates described herein. By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered inter-nucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In exemplary aspects, the nucleic acids of the present disclosure are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

In some aspects, the nucleic acid encodes only a portion of the antibodies, antigen-binding fragments, polypeptides, or conjugates. For example, when the conjugate comprises a polymer, which does not comprise amino acids and thus is not encoded by a nucleic acid, the nucleic acid encodes only the part of the conjugate which can be encoded by a nucleic acid. In exemplary embodiments, the nucleic acid comprises a nucleotide sequence encoding a polypeptide comprising an amino acid sequence comprising each of SEQ ID NOs: 1-6. In exemplary aspects, the nucleic acid comprises a nucleotide sequence encoding an amino acid sequence comprising each of SEQ ID NOs: 1-6 wherein one or more amino acids are present between each of SEQ ID NOs: 1-6. In exemplary aspects, the nucleic acid comprises the nucleotide sequence of SEQ ID NO: 7 and/or SEQ ID NO: 8.

In exemplary embodiments, the nucleic acid comprises a nucleotide sequence encoding a polypeptide comprising an amino acid sequence comprising each of SEQ ID NOs: 21-26. In exemplary aspects, the nucleic acid comprises a nucleotide sequence encoding an amino acid sequence comprising each of SEQ ID NOs: 21-26 wherein one or more amino acids are present between each of SEQ ID NOs: 21-26. In exemplary embodiments, the nucleic acid comprises a nucleotide sequence encoding a polypeptide comprising an amino acid sequence of SEQ ID NO: 27 and/or SEQ ID NO: 28.

In exemplary embodiments, the nucleic acid comprises a nucleotide sequence encoding a polypeptide comprising an amino acid sequence comprising each of SEQ ID NOs: 29-34. In exemplary aspects, the nucleic acid comprises a nucleotide sequence encoding an amino acid sequence comprising each of SEQ ID NOs: 29-34 wherein one or more amino acids are present between each of SEQ ID NOs: 29-34. In exemplary embodiments, the nucleic acid comprises a nucleotide sequence encoding a polypeptide comprising an amino acid sequence of SEQ ID NO: 35 and/or SEQ ID NO: 36.

In exemplary embodiments, the nucleic acid comprises a nucleotide sequence encoding a polypeptide comprising an amino acid sequence comprising each of SEQ ID NOs: 37-42. In exemplary aspects, the nucleic acid comprises a nucleotide sequence encoding an amino acid sequence comprising each of SEQ ID NOs: 37-42 wherein one or more amino acids are present between each of SEQ ID NOs: 37-42. In exemplary embodiments, the nucleic acid comprises a nucleotide sequence encoding a polypeptide comprising an amino acid sequence of SEQ ID NO: 43 and/or SEQ ID NO: 44.

In exemplary embodiments, the nucleic acid comprises a nucleotide sequence encoding a polypeptide comprising an amino acid sequence comprising each of SEQ ID NOs: 45-50. In exemplary aspects, the nucleic acid comprises a nucleotide sequence encoding an amino acid sequence comprising each of SEQ ID NOs: 45-50 wherein one or more amino acids are present between each of SEQ ID NOs: 45-50. In exemplary embodiments, the nucleic acid comprises a nucleotide sequence encoding a polypeptide comprising an amino acid sequence of SEQ ID NO: 51 and/or SEQ ID NO: 52.

In exemplary embodiments, the nucleic acid comprises a nucleotide sequence encoding a polypeptide comprising an amino acid sequence comprising each of SEQ ID NOs: 58-63. In exemplary aspects, the nucleic acid comprises a nucleotide sequence encoding an amino acid sequence comprising each of SEQ ID NOs: 58-63 wherein one or more amino acids are present between each of SEQ ID NOs: 58-63. In exemplary embodiments, the nucleic acid comprises a nucleotide sequence encoding a polypeptide comprising an amino acid sequence of SEQ ID NO: 64 and/or SEQ ID NO: 65.

In exemplary embodiments, the nucleic acid comprises a nucleotide sequence encoding a polypeptide comprising an amino acid sequence comprising each of SEQ ID NOs: 106-111. In exemplary aspects, the nucleic acid comprises a nucleotide sequence encoding an amino acid sequence comprising each of SEQ ID NOs: 106-111 wherein one or more amino acids are present between each of SEQ ID NOs: 106-111. In exemplary embodiments, the nucleic acid comprises a nucleotide sequence encoding a polypeptide comprising an amino acid sequence of SEQ ID NO: 67 and/or SEQ ID NO: 69. In exemplary aspects, the nucleic acid comprises the nucleotide sequence of SEQ ID NO: 66 and/or SEQ ID NO: 68.

The nucleic acids are useful in e.g., methods of recombinant production of the binding constructs of the invention.

### Recombinant Expression Vector

The nucleic acids of the invention can be incorporated into a recombinant expression vector, or “vector”. In this regard, the invention provides recombinant expression vectors or “vectors” comprising any of the nucleic acids of the invention. For purposes herein, the term “recombinant expression vector” or “vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the altered nucleotides or non-naturally occurring internucleotide linkages do not hinder the transcription or replication of the vector.

The recombinant expression vector of the invention can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGTIO, λGTI 1, λZapII (Stratagene), λEMBL4, and λNMI 149, also can be used. Examples of plant expression vectors include pBIOl, pBI101.2, pBI101.3, pBI121 and pBIN19 (Clontech). Examples of animal expression vectors include pEUK-Cl, pMAM and pMAMneo (Clontech). Preferably, the recombinant expression vector is a viral vector, e.g., a retroviral vector.

The recombinant expression vectors of the invention can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

Desirably, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The recombinant expression vector can comprise a native or non-non-native promoter operably linked to the nucleotide sequence encoding the polypeptide (including functional portions and functional variants thereof), or to the nucleotide sequence which is complementary to or which hybridizes to the nucleotide sequence encoding the polypeptide. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

### Host Cells

The invention further provides a host cell comprising any of the nucleic acids or recombinant expression vectors described herein. As used herein, the term “host cell” refers to any type of cell that can contain and express the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5a cell. For purposes of producing a recombinant polypeptide the host cell is preferably a mammalian cell, e.g., a CHO cell.

### Kits

Provided herein are kits comprising any one or more of the antibody or antigen-binding fragment or polypeptide or conjugate or nucleic acid or vector or host cell, as described herein, or a combination of any of the foregoing. In exemplary aspects, the binding construct is provided in the kit in a predetermined amount or concentration. For example, the kit may be a detection kit comprising a predetermined amount of the binding construct for detecting ZIKV in a sample. In exemplary embodiments, the one or more of the binding constructs of the present disclosure is provided in the kit in an aqueous solution. In exemplary aspects, the aqueous solution is provided to the end-user on dry ice. In some aspects, the aqueous solution is provided to the end-user separately from the other components of the kit. In exemplary embodiments, the binding constructs of the present disclosure are provided in the kit in a lyophilized or other freeze-dried form. In exemplary aspects, the binding constructs of the present disclosures are provided in the kit in a frozen or cryopreserved form.

In exemplary aspects, the kit comprises a solid support, and in exemplary aspects the antibody or antigen-binding fragment or polypeptide or conjugate is pre-coated onto the solid support. In exemplary aspects, the kit comprises a solid support selected from the group consisting of a tube, a dish, a flask, a bag, a plate (e.g., a microtiter plate), a membrane, a filter, a bead, a fiber, a probe, and the like. In exemplary aspects, the solid support is made of a polymer. In exemplary aspects, the solid support is made of agarose, cellulose, dextran, polyacrylamide, latex, or controlled pore glass. In exemplary aspects, the solid support is made of polyvinyl difluoride (PVDF), nitrocellulose, nylon 66, protran nitrocellulose, or paper. In exemplary aspects, the membrane is one of the Immobilon®, Protran®, QuickDraw®, Westran®, Whatman® or Hybond® membranes (Sigma-Aldrich, St. Louis, Mo.). In exemplary aspects, the solid support is a polymer bead, a microtiter plate, a membrane or a filter. In exemplary aspects, the kit comprises a solid support comprising pre-aliquoted amounts of the antibody or antigen-binding fragment or polypeptide or conjugate.

In exemplary aspects, the kit comprises a capture molecule which binds to a Zika virus. In exemplary aspects, the capture molecule is bound to the solid support. In exemplary aspects, the capture molecule is an antibody or an antigen-binding fragment thereof. In particular aspects, the capture molecule is an antibody or antigen-binding fragment comprising the amino acid sequences of SEQ ID NOs: 1-6, 21-26, 29-34, 37-42, 45-50, 58-63 or 106-111. In particular aspects, the capture molecule is an antibody or antigen-binding fragment comprising the amino acid sequence of SEQ ID NO: 9 and/or SEQ ID NO: 10, SEQ ID NO: 27 and/or SEQ ID NO: 28, SEQ ID NO: 35 and/or SEQ ID NO: 36, SEQ ID NO: 43 and/or SEQ ID NO: 44, SEQ ID NO: 51 and/or SEQ ID NO: 52, SEQ ID NO: 64 and/or SEQ ID NO: 65, or SEQ ID NO: 67 and/or SEQ ID NO: 69. In exemplary aspects, the capture molecule is a lectin, e.g., concanavalin A.

In exemplary aspects, the kit comprises additional reagents, substrates, solvents, buffers, diluents, etc., used in the detection methods described herein. In exemplary aspects, any one or more of the additional components are provided in the kit in a predetermined amount, e.g., the amount necessary and suitable for a detection assay. In exemplary aspects, the kit comprises a blocking agent, such as, for example, a solution comprising bovine serum albumin (BSA). In exemplary aspects, the kit comprises a wash buffer, such as, for example, phosphate buffered saline or TRIS buffer. In exemplary aspects, the kit comprises a detecting agent. Suitable detecting agents are known in the art and described herein. See, e.g., the section herein entitled “Conjugates: Detecting Agents”. In exemplary aspects, the detecting agent comprises a secondary antibody linked to a detectable label. The detectable label, in some aspects, is an enzyme, e.g., horseradish peroxidase (HRP). In exemplary aspects, the kit comprises a substrate of the enzyme, and in some aspects, the substrate is a chromogenic substrate. Suitable substrates of the enzyme of the detectable label are known in the art and include but is not limited to 2,2′-azino-bis(3-ethylbenzothiazoline-6-sulphonic acid) (ABTS), o-phenylenediamine dihydrochloride (OPD), AmplexRed, 3,3′-Diaminobenzidine (DAB), aminoethyl carbazole (AEC), 3,3′,5,5′-Tetramethylbenzidine (TMB), Homovanillic acid, and Luminol. In exemplary aspects, the secondary antibody of the detecting agent binds to the antibody or antigen-binding fragment or polypeptide of the present disclosure, which binds to a Zika virus (ZIKV) protein and does not bind to a Dengue virus (DENV) protein. In exemplary aspects, the secondary antibody is an antibody that binds to the Fc of ZIKV-specific antibody. In exemplary aspects, the ZIKV-specific antibody comprises a Rhesus monkey constant region (e.g., Fc) and the secondary antibody is one that binds to Rhesus monkey constant region (e.g., Fc).

In exemplary aspects, the kit comprises reagents and materials for an ELISA, e.g., a sandwich ELISA. In exemplary aspects, the kit comprises a ZIKV-specific antibody of the present disclosures (e.g., one comprising the amino acid sequence of SEQ ID NOs: 1-6, 21-26, 29-34, 37-42, 45-50, 58-63, or 106-111) as a detection antibody, a solid support (e.g., a microtiter plate or nitrocellulose) coated with a capture molecule and blocked with a blocking agent, e.g., BSA. In exemplary aspects, the kit comprises a detecting agent. In exemplary aspects, the detection antibody comprises the amino acid sequences of SEQ ID NOs: 1-6 and the heavy and light chain constant regions of a Rhesus monkey antibody (e.g., SEQ ID NOs 19 and 20). In exemplary aspects, the capture molecule is another antibody of the present disclosure, e.g., one comprising the amino acid sequences of SEQ ID NOs: 29-34. In exemplary aspects, the detecting agent is a secondary antibody which binds to the Fc of a Rhesus monkey antibody and binds to the Fc of the detection antibody and the secondary antibody is conjugated to a detectable label. In exemplary aspects, the detectable label is horseradish peroxidase (HRP). In exemplary aspects, the kit comprises a chromogenic substrate for HRP. In exemplary aspects, a positive control and/or a negative control is provided for the ELISA.

In alternative aspects, the kit comprises reagents and materials for another immunoassay. In exemplary aspects, the kit comprises a ZIKV-specific antibody of the present disclosure (e.g., one comprising the amino acid sequence of SEQ ID NOs: 1-6, 21-26, 29-34, 37-42, 45-50, 58-63, or 106-111) as a detection antibody, a solid support (e.g., a microtiter plate or nitrocellulose) coated with a capture molecule. In exemplary aspects, the kit comprises a detecting agent. In exemplary aspects, the detection antibody comprises the amino acid sequences of SEQ ID NOs: 1-6 or SEQ ID NOs: 29-34 or SEQ ID NOs: 58-63 in addition to the heavy and light chain constant regions of a Rhesus monkey antibody (e.g., SEQ ID NOs 19 and 20). In exemplary aspects, the capture molecule is a lectin, e.g., Concanavalin A. In exemplary aspects, the detecting agent is a secondary antibody which binds to the Fc of a Rhesus monkey antibody and binds to the Fc of the detection antibody and the secondary antibody is conjugated to a detectable label. In exemplary aspects, the detectable label is horseradish peroxidase (HRP). In exemplary aspects, the kit comprises a chromogenic substrate for HRP. In exemplary aspects, a positive control and/or a negative control is provided for the immunoassay.

### Assay Systems

The present disclosure provides an assay system. In exemplary aspects, the assay system is suitable for detecting a ZIKV infection and ZIKV immunity in a subject. Without being bound to a particular theory, the detection of ZIKV-specific antibodies in the sample of a subject represents prior exposure to ZIKV and hence ZIKV immunity. In exemplary aspects, the assay system is a lateral flow assay system. In exemplary aspects, the assay system is an immunochromatographic assay system. Lateral flow assay systems are known in the art. See, e.g., Grant et al., Vaccine 34(46): 5656-5663 (2016); and Cross et al., J Infect Dis 214(suppl 3):5210-5217 (2016).

In exemplary aspects, the assay system comprises a porous matrix comprising at least three zones, Zone A, Zone B, and Zone C, wherein Zone A comprises an antibody or antigen-binding fragment thereof as described herein, wherein the antibody or antigen binding fragment thereof is not bound to a Zika virus, Zone B comprises an antibody or antigen-binding fragment thereof as described herein, wherein the antibody or antigen binding fragment thereof is bound to a Zika virus, and Zone C comprises a secondary antibody which binds the antibody or antigen-binding fragment thereof of Zone A and Zone B, optionally, wherein the secondary antibody binds to the Fc of the antibody of Zone A and Zone B. In exemplary aspects, Zone A is purposed for testing for an active ZIKV infection in the subject, Zone B is purposed for testing for ZIKV immunity or prior exposure to ZIKV, and Zone C is purposed as a control. In exemplary aspects, Zones A to C are arranged along a horizontal axis. In exemplary aspects, each of Zones A, B, and C is flanked by an intervening zone of the porous matrix lacking the antibody or antigen-binding fragment thereof.

In exemplary aspects, the assay system further comprises a sample application pad, a particle conjugate zone, a wick, and/or a backing. In exemplary aspects, the porous matrix comprising Zones A, B, and C, the sample application pad, the particle conjugate zone, and the wick are arranged along a horizontal axis. In some aspects, the horizontal axis is the same as the horizontal axis along which Zones A to C are arranged. In exemplary aspects, the assay system is arranged such that the sample application pad and the wick are located at opposite ends of the assay system along the horizontal axis. In some aspects, the particle conjugate zone is flanked by the sample application pad and the porous matrix comprising Zones A, B, and C. In some aspects, the porous matrix is flanked by the particle conjugate and the wick. In exemplary aspects, the backing is positioned below the porous matrix, the sample application pad, the particle conjugate zone, and the wick. In some aspects, the backing provides a physical support for the sample application pad, the particle conjugate zone, the porous matrix, and the wick.

In exemplary aspects, the particle conjugate zone is bound to a conjugate comprising an antibody or antigen-binding fragment or polypeptide as described herein, bound to an element or polymer. In exemplary aspects, the element is a gold particle or the polymer is polystyrene. In exemplary aspects, the conjugate comprises an antibody as described herein. In some aspects, the antibody of the conjugate comprises any one or more of SEQ ID NOs: 1-6 and SEQ ID NOs: 11-16. In exemplary aspects, the antibody of the conjugate comprises SEQ ID NO: 9 and/or SEQ ID NO: 10.

In exemplary aspects, each of Zone A and Zone B is bound to an antibody as described herein. In some aspects, the antibody bound to Zone A and Zone B comprises any one or more of SEQ ID NOs: 29-34. In exemplary aspects, the antibody of the conjugate comprises SEQ ID NO: 35 and/or SEQ ID NO: 36. In exemplary aspects, the antibody bound to each of Zone A and Zone B has an Fc which is the same as the Fc of the antibody of the conjugate bound to the particle conjugate zone. In exemplary aspects, the Fc of the antibody bound to Zone A and Zone B and the Fc of the antibody of the conjugate comprises a non-human Fc. In exemplary aspects, the non-human Fc is an Fc of a mouse, goat, rabbit, or non-human primate antibody. In exemplary aspects, the non-human Fc is an Fc of a Rhesus monkey antibody.

In exemplary aspects, the porous matrix comprises a solid support. In exemplary aspects, the solid support is a filter or a membrane. In exemplary aspects, the porous matrix comprises nitrocellulose or polyvinylidene fluoride (PVDF). In exemplary aspects, the sample application pad comprises cellulose or glass fiber. In exemplary aspects, the wick comprises nitrocellulose.

### Detection Methods

Binding constructs provided herein are useful in, e.g., detection methods that allow for unambiguous or specific detection of ZIKV in samples, e.g., clinical samples comprising, e.g., ZIKV and DENV and/or another flavivirus. The binding constructs can be used in any antibody-based assay or technique or any immunoassay known in the art, such as, but not limited to, radioimmunoassay (RIA), magnetic immunoassay (MIA), immunocytochemical (ICC) assays, immunohistochemical (IHC) assays, immunofluorescent assays, ELISA, EIA, ELISPOT, enzyme multiplied immunoassay, radiobinding assay, Western blotting, immunoprecipitation, dot blots, flow cytometry, real-time immunoquantitative PCR, protein microarrays and the like. See, e.g., The Immunoassay Handbook (Fourth Edition); Theory and Applications of Ligand Binding, ELISA and Related Techniques, ed. Wild, Elsevier Ltd. (Oxford, UK) 2013, Green and Sambrook, Molecular Cloning: A Laboratory Manual, 4^{th} ed., Cold Spring Harbor Laboratory Press (Cold Spring Harbor, N.Y.) 2012, and Immunoassay, Diamandis and Christopolous, Academic Press 1996.

Accordingly, provided herein are uses of the binding construct (e.g., antibody or antigen-binding fragment, polypeptide, or conjugate), nucleic acid, vector, host cell, and/or kit described herein for detecting ZIKV in a sample. The present disclosure provides methods of detecting ZIKV in a sample obtained from a subject. In exemplary embodiments, the method comprises (i) contacting the sample with a binding construct (e.g., an antibody or antigen-binding fragment or polypeptide or conjugate) as described herein to form a complex (e.g., an immunocomplex) comprising ZIKV and the binding construct (e.g., antibody, antigen-binding fragment, polypeptide, or conjugate), and (ii) detecting the complex. When the complex is detected, it is determined that the sample, and thus the subject, is positive for ZIKV, e.g., the subject is infected with ZIKV.

In exemplary embodiments, detecting the complex comprises detecting a signal of a detecting agent. In exemplary embodiments, the signal is based on enzymatic activity, radioactivity, chromogenic activity, and/or binding activity. In exemplary embodiments, the signal is radioactive, chromogenic, colorimetric, fluorometric, chemiluminescent, enhanced chemiluminescent, direct fluorescent, time-resolved fluorescent, direct chemiluminescent, phosphorescent, enzymatic, or based on binding of a micro- or nanoparticle, streptavidin/avidin-biotin and protein A. In exemplary embodiments, the detecting agent comprises an enzyme, a radioactive isotope, a DNA reporter, a chromogenic or fluorogenic reporter, an electrochemiluminescent tag. In exemplary embodiments, detecting the complex comprises carrying out surface plasmon resonance to detect the complex or measuring change in resistance on an electrode (as FIX ZIKV binds to the antibody, antigen-binding fragment, polypeptide, or conjugate). See, Gonzalez-Diaz et al., “Plasmonic Au/Co/Au nanosandwiches with Enhanced Magneto-Optical Activity” Small 4(2): 202-5 (2008) and Tsekenis (2008). “Label-less immunosensor assay for myelin basic protein based upon an ac impedance protocol.” Analytical Chemistry 80 (6): 2058-62 (2008). In exemplary aspects, the enzyme is horseradish peroxidase (HRP), alkaline phosphatase (AP), glucose oxidase, or beta-galactosidase. In exemplary aspects, the enzyme is exposed to reagents which cause them to chemiluminesce or produce light. In exemplary aspects, the radioisotope is 1125. In exemplary aspects, the DNA reporter is a DNA probe. See, e.g., Rajkovic, “Immunoquantitative real-time PCR for detection and quantification of Staphylococcus aureus enterotoxin B in foods.” Applied and Environmental Microbiology 72 (10): 6593-9 (2006); and Gofflot “Immuno-quantitative polymerase chain reaction for detection and quantitation of prion protein.” Journal of Immunoassay and Immunochemistry 25 (3): 241-58 (2004). In exemplary aspects, the fluorogenic reporter is phycoerythrin (PE) e.g., B-PE, R-PE, or allophycocyanin (APC).

In exemplary embodiments, the antibody or antigen-binding fragment or polypeptide is conjugated to a detecting agent. In exemplary embodiments, the conjugate comprises a detecting agent. In exemplary embodiments, the antibody or antigen-binding fragment or polypeptide is not conjugated to a detecting agent or the conjugate does not comprises a detecting agent. In such exemplary embodiments, the methods comprise contacting the sample with a secondary antibody comprising a detecting agent, wherein the secondary antibody binds to the antibody or antigen-binding fragment or polypeptide or conjugate. The secondary antibody may be any antibody of any isotype or class, provided that the secondary antibody will bind to the anti-ZIKV antibody, antigen-binding fragment thereof, polypeptide or conjugate.

In exemplary embodiments, the antibody or antigen-binding fragment or polypeptide is conjugated to a solid support. In exemplary embodiments, the conjugate comprises a solid support. For example, the solid support is selected from the group consisting of a tube, a dish, a flask, a bag, a plate (e.g., a microtiter plate), a membrane, a filter, a bead, a fiber, a probe, and the like. In exemplary aspects, the solid support is made of a polymer. In exemplary aspects, the solid support is made of agarose, cellulose, dextran, polyacrylamide, latex, or controlled pore glass. In exemplary aspects, the solid support is made of agarose. In exemplary aspects, the solid support is made of polyvinyl difluoride (PVDF), nitrocellulose, nylon 66, protran nitrocellulose, or paper. In exemplary aspects, the membrane is one of the Immobilon®, Protran®, QuickDraw®, Westran®, Whatman® or Hybond® membranes (Sigma-Aldrich, St. Louis, Mo.). In exemplary aspects, the solid support is a polymer bead, a magnetic or paramagnetic bead, a microtiter plate, a membrane or a filter.

The present disclosure provides a method of detecting a Zika virus (ZIKV) infection in a subject. In exemplary embodiments, the method comprises (i) contacting a sample obtained from the subject with an antibody, antigen-binding fragment, or polypeptide as described herein, thereby forming a test mixture, and (ii) assaying the test mixture for a complex comprising ZIKV bound to the antibody, antigen-binding fragment, or polypeptide, wherein, when the complex is present in the test mixture, the subject is determined as having a ZIKV infection. In exemplary aspects, the sample is selected from the group consisting of blood, plasma, serum, urine, semen, lacrimal fluid, saliva, or tissue fluids.

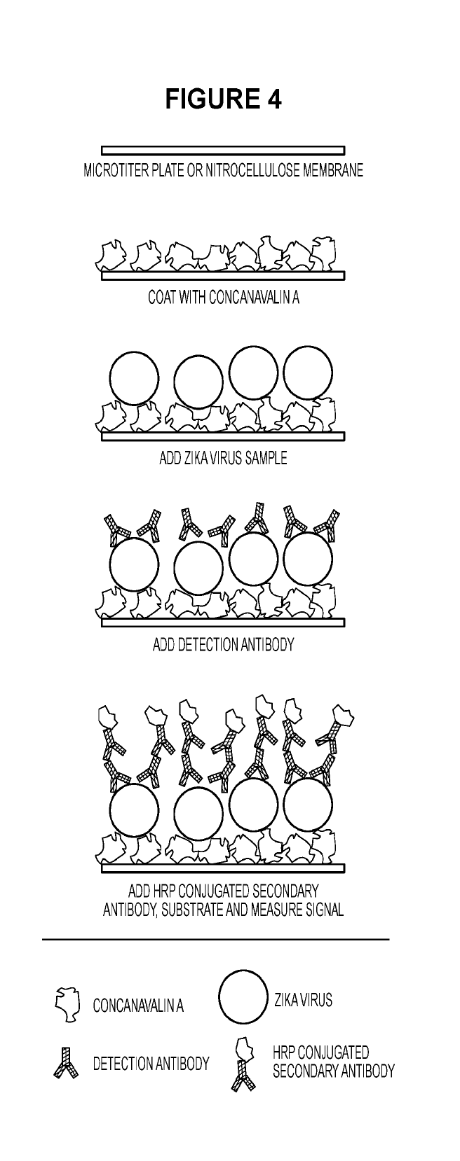

In exemplary aspects, the method of detecting a ZIKV infection in a subject comprises comprise (i) adding a sample obtained from the subject to a solid support bound to a capture molecule that binds to ZIKV, (ii) adding a detection antibody comprising an antibody, antigen-binding fragment, or polypeptide as described herein, (iii) adding a detection agent which binds to the detection antibody, and (iv) assaying for a signal from the detection agent, wherein, when the signal is detected, the subject is determined as having a ZIKV infection. In exemplary aspects, the method is carried out with a kit as described herein. In exemplary aspects, the method is a sandwich ELISA. In exemplary aspects, one or more areas of the solid support not bound to the capture molecule is bound to a blocking agent, optionally, bovine serum albumin. In exemplary aspects, the capture molecule is a lectin which binds to ZIKV. In exemplary aspects, the capture molecule is concanavalin A. In alternative aspects, the capture molecule is an antibody antigen-binding fragment, or polypeptide as described herein. In exemplary aspects, the capture molecule comprises one or more (e.g., two, three, four, five, six) of the amino acid sequences of SEQ ID NOs: 29-34. In exemplary aspects, the capture molecule comprises SEQ ID NOs: 35 and/or 36. In exemplary aspects, the detection antibody comprises one or more (e.g., two, three, four, five, six) of the amino acid sequences of SEQ ID NOs: 1-6. In exemplary aspects, the detection antibody comprises SEQ ID NOs: 9 and/or 10. In exemplary aspects, the detection antibody comprises one or more (e.g., two, three, four, five, six) of the amino acid sequences of SEQ ID NOs: 29-34. In exemplary aspects, the detection antibody comprises SEQ ID NOs: 35 and/or 36. In exemplary aspects, the detection antibody comprises one or more (e.g., two, three, four, five, six) of the amino acid sequences of SEQ ID NOs: 58-63. In exemplary aspects, the detection antibody comprises SEQ ID NOs: 64 and/or 65. In exemplary aspects, the detection antibody comprises one or more (e.g., two, three, four, five, six) of the amino acid sequences of SEQ ID NOs: 106-111. In exemplary aspects, the detection antibody comprises SEQ ID NOs: 67 and/or 69. In exemplary aspects, the detection antibody comprises a constant region which is recognized by a secondary antibody and the detection agent is the secondary antibody conjugated to an enzyme. In exemplary aspects, the enzyme is HRP. In exemplary aspects, the method comprises adding substrate to the detecting agent and detecting the signal produced upon adding the substrate. In exemplary aspects, the method is as essentially shown in FIG. 4.

The present disclosure also provides a method of detecting Zika virus (ZIKV) immunity in a subject. The method in some aspects is a method of detecting ZIKV-specific antibodies made by the subject being tested, the presence of such antibodies indicating that the subject has had a previous exposure to ZIKV. Thus, the present disclosure provides a method of determining whether a subject has had a prior infection to ZIKV or a prior exposure to ZIKV. The present disclosure provides a method of detecting ZIKV antibodies in a sample obtained from a subject. In exemplary aspects, these methods comprise (i) adding a blood, plasma, or serum sample obtained from the subject to a solid support bound to a capture molecule that binds to ZIKV, (ii) adding a detection antibody comprising an antibody, antigen-binding fragment, or polypeptide as described herein, (iii) adding a detection agent which binds to the detection antibody, and (iv) assaying for a signal from the detection agent, wherein, when the signal is detected, the subject is determined as not having ZIKV immunity (or as not having a previous exposure to ZIKV or prior ZIKV infection) and, when the signal is not detected, the subject is determined as having ZIKV immunity (or as having a previous exposure to ZIKV or prior ZIKV infection).

The present disclosure also provides a method of detecting Zika virus (ZIKV) exposure in a subject. The method in some aspects is a method of detecting ZIKV-specific antibodies made by the subject being tested, the presence of such antibodies indicating that the subject has had a previous exposure to ZIKV. In exemplary aspects, the method comprises (i) adding a blood, plasma, or serum sample obtained from the subject to a solid support bound to a capture molecule that binds to ZIKV or ZIKV-derived antigens, (ii) adding a detection antibody comprising an antibody, antigen-binding fragment, or polypeptide as described herein, (iii) adding a detection agent which binds to the detection antibody, and (iv) assaying for a signal from the detection agent, wherein, when the signal is detected, the subject is determined as not having previous ZIKV exposure and, when the signal is not detected, the subject is determined as having a previous ZIKV exposure. In exemplary aspects, the method further comprises a wash step. The wash step in some aspects is after step (i), after step (ii), and/or after step (iii) of the method. In exemplary aspects, the method is carried out with a kit as described herein. In exemplary aspects, the method is a sandwich ELISA. In exemplary aspects, one or more areas of the solid support not bound to the capture molecule is bound to a blocking agent, optionally, bovine serum albumin.

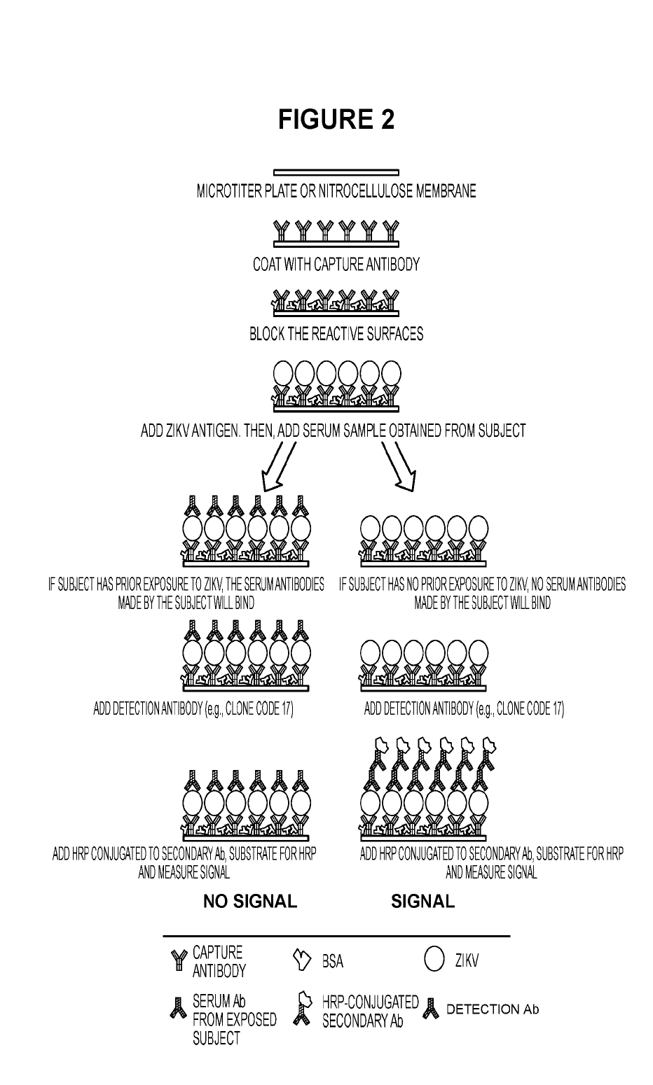

In exemplary aspects, the methods further comprise a wash step. The wash step in some aspects is after the step of adding a blood, plasma, or serum sample obtained from the subject to a solid support bound to a capture molecule that binds to ZIKV, after the step of adding a detection antibody comprising an antibody, antigen-binding fragment, or polypeptide as described herein, or after the step of adding a detection agent which binds to the detection antibody, or a combination thereof. In exemplary aspects, the method is carried out with a kit as described herein. In exemplary aspects, the method is a sandwich ELISA. In exemplary aspects, one or more areas of the solid support not bound to the capture molecule is bound to a blocking agent, optionally, bovine serum albumin. In exemplary aspects, the capture molecule is an antibody antigen-binding fragment, or polypeptide as described herein. In exemplary aspects, the capture molecule comprises one or more (e.g., two, three, four, five, six) of the amino acid sequences of SEQ ID NOs: 29-34. In exemplary aspects, the capture molecule comprises SEQ ID NOs: 35 and/or 36. In exemplary aspects, the detection antibody comprises one or more (e.g., two, three, four, five, six) of the amino acid sequences of SEQ ID NOs: 1-6. In exemplary aspects, the detection antibody comprises SEQ ID NOs: 9 and/or 10. In exemplary aspects, the detection antibody comprises one or more (e.g., two, three, four, five, six) of the amino acid sequences of SEQ ID NOs: 29-34. In exemplary aspects, the detection antibody comprises SEQ ID NOs: 35 and/or 36. In exemplary aspects, the detection antibody comprises one or more (e.g., two, three, four, five, six) of the amino acid sequences of SEQ ID NOs: 58-63. In exemplary aspects, the detection antibody comprises SEQ ID NOs: 64 and/or 65. In exemplary aspects, the detection antibody comprises one or more (e.g., two, three, four, five, six) of the amino acid sequences of SEQ ID NOs: 106-111. In exemplary aspects, the detection antibody comprises SEQ ID NOs: 67 and/or 69. In exemplary aspects, the detection antibody comprises a constant region which is recognized by a secondary antibody and the detection agent is the secondary antibody conjugated to an enzyme. In exemplary aspects, the enzyme is HRP. In exemplary aspects, the method comprises adding substrate to the detecting agent and detecting the signal produced upon adding the substrate. In exemplary aspects, the method is as essentially shown in FIG. 2.

Additionally provided herein is a method of detecting a Zika virus (ZIKV) infection and ZIKV immunity in a subject. In exemplary embodiments, the method comprises adding a sample obtained from a subject to the assay system as described herein. In exemplary aspects, when the assay system exhibits a single band in Zone C, the subject is determined as having neither a ZIKV infection nor ZIKV immunity, when the assay system exhibits a band in each of Zone A and Zone B, the subject is determined as having both a ZIKV infection and ZIKV immunity, and when the assay system exhibits a band in Zone B and a band is absent in Zone A, the subject is determined as not having a ZIKV infection but having ZIKV immunity. In exemplary aspects, the sample is blood, plasma, serum, urine, semen, lacrimal fluid, saliva, or tissue fluids.

### Vaccine Efficacy

The present disclosure further provides a method of assessing efficacy of a Zika virus (ZIKV) vaccine in a subject who has received a ZIKV vaccine. In exemplary aspects, the method comprises adding a sample obtained from the subject to the assay system as described herein, wherein, when the assay system exhibits (i) a band in each of Zone A and Zone B or (ii) a band in Zone B and a band is absent in Zone A, the ZIKV vaccine is determined as effective in the subject, and when the assay system exhibits a single band in Zone C, the ZIKV vaccine is determined as ineffective in the subject. In exemplary aspects, the method comprises (i) adding a blood, plasma, or serum sample obtained from the subject to a solid support bound to a capture molecule that binds to ZIKV, (ii) adding a detection antibody comprising an antibody, antigen-binding fragment, or polypeptide described herein, (iii) adding a detection agent which binds to the detection antibody, and (iv) assaying for a signal from the detection agent, wherein, when the signal is detected, the vaccine is determined as ineffective in the subject, and, when the signal is not detected, the vaccine is determined as effective in the subject.

### Pharmaceutical Compositions and Routes of Administration

In exemplary embodiments, the binding constructs (e.g., an antibody or antigen-binding fragment), polypeptides, nucleic acids, expression vectors, host cells, and conjugates of the present disclosure are provided as part of a composition. Accordingly, the present disclosure provides a composition comprising any one or more of the binding constructs (e.g., an antibody or antigen-binding fragment), polypeptides, nucleic acids, expression vectors, host cells, and conjugates of the present disclosure, or a combination thereof. In accordance with some embodiments, the composition is a pharmaceutical composition comprising any one or more of the binding constructs (e.g., an antibody or antigen-binding fragment), polypeptides, nucleic acids, expression vectors, host cells, and conjugates of the present disclosure, or a combination thereof, and a pharmaceutically acceptable carrier, diluent, or excipient.

The pharmaceutical compositions may be formulated to achieve a physiologically compatible pH. In some embodiments, the pH of the pharmaceutical composition may be at least 5, at least 5.5, at least 6, at least 6.5, at least 7, at least 7.5, at least 8, at least 8.5, at least 9, at least 9.5, at least 10, or at least 10.5 up to and including pH 11, depending on the formulation and route of administration, for example between 4 and 7, or 4.5 and 5.5. In illustrative embodiments, the pharmaceutical compositions may comprise buffering agents to achieve a physiological compatible pH. The buffering agents may include any compounds capable of buffering at the desired pH such as, for example, phosphate buffers (e.g., PBS), triethanolamine, Tris, bicine, TAPS, tricine, HEPES, TES, MOPS, PIPES, cacodylate, MES, acetate, citrate, succinate, histidine or other pharmaceutically acceptable buffers.

In various embodiments, the physiologically and pharmaceutically acceptable carrier can include any of the well-known components useful for immunization. The carrier can facilitate or enhance an immune response to an antigen administered in a vaccine. The cell formulations can contain buffers to maintain a preferred pH range, salts or other components that present an antigen to an individual in a composition that stimulates an immune response to the antigen. The physiologically acceptable carrier also can contain one or more adjuvants that enhance the immune response to an antigen. Pharmaceutically acceptable carriers include, for example, pharmaceutically acceptable solvents, suspending agents, or any other pharmacologically inert vehicles for delivering compounds to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water, saline solution, binding agents (e.g., polyvinylpyrrolidone or hydroxypropyl methylcellulose); fillers (e.g., lactose or dextrose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate). Compositions can be formulated for subcutaneous, intramuscular, or intradermal administration, or in any manner acceptable for administration.

An adjuvant refers to a substance which, when added to an immunogenic agent such as a cell containing the expression vector system of the invention, nonspecifically enhances or potentiates an immune response to the agent in the recipient host upon exposure to the mixture. Adjuvants can include, for example, oil-in-water emulsions, water-in oil emulsions, alum (aluminum salts), liposomes and microparticles, such as, polysytrene, starch, polyphosphazene and polylactide/polyglycosides.

Adjuvants can also include, for example, squalene mixtures (SAF-I), muramyl peptide, saponin derivatives, mycobacterium cell wall preparations, monophosphoryl lipid A, mycolic acid derivatives, nonionic block copolymer surfactants, Quil A, cholera toxin B subunit, polyphosphazene and derivatives, and immunostimulating complexes (ISCOMs) such as those described by Takahashi et al., Nature 1990, 344:873-875. For veterinary use and for production of antibodies in animals, mitogenic components of Freund's adjuvant (both complete and incomplete) can be used. In humans, Incomplete Freund's Adjuvant (IFA) is a useful adjuvant. Various appropriate adjuvants are well known in the art (see, for example, Warren and Chedid, CRC Critical Reviews in Immunology 1988, 8:83; and Allison and Byars, in Vaccines: New Approaches to Immunological Problems, 1992, Ellis, ed., Butterworth-Heinemann, Boston). Additional adjuvants include, for example, bacille Calmett-Guerin (BCG), DETOX (containing cell wall skeleton of Mycobacterium phlei (CWS) and monophosphoryl lipid A from Salmonella minnesota (MPL)), and the like (see, for example, Hoover et al., J Clin Oncol 1993, 11:390; and Woodlock et al., J Immunother 1999, 22:251-259).

In exemplary aspects, the pharmaceutical compositions may be formulated for administration to the subject via parenteral, intravenous, intramuscular, subcutaneous, sublingual, nasal, inhalation, vaginal, rectal, oral, or topical administration. In exemplary aspects, the pharmaceutical compositions is formulated for parenteral administration. Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The analog of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

The parenteral formulations will typically contain from about 0.5% to about 25% by weight of the analog of the present disclosure in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

When the pharmaceutical composition comprises cells, the pharmaceutical composition may be administered to the subject through any suitable method known in the art, including, for example, perfusions, infusions and injections. See, e.g., Burch et al., Clin Cancer Res 6(6): 2175-2182 (2000), Dudley et al., J Clin Oncol 26(32): 5233-5239 (2008); Khan et al., Cell Transplant 19:409-418 (2010); Gridelli et al., Liver Transpl 18:226-237 (2012)).

### Therapeutic Methods

Because some of the binding constructs of the present disclosure are neutralizing antibodies, the present disclosure provides methods of treating a ZIKV infection in a subject. In exemplary aspects, the method comprises administering to the subject a binding construct (e.g., an antibody or antigen-binding fragment thereof) in, e.g., an amount to treat or prevent the ZIKV injection in the subject.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a ZIKV infection of the present disclosure can provide any amount or any level of treatment. Furthermore, the treatment provided by the method of the present disclosure may include treatment of one or more conditions or symptoms or signs of the infection, being treated. Also, the treatment provided by the methods of the present disclosure may encompass slowing the progression of the infection. For example, the methods can treat the infection by virtue of eliciting an immune response against ZIKV, stimulating or activating CD8+ T cells specific for ZIKV to proliferate, stimulating or activating the classical complement pathway, and the like.

As used herein, the term “prevent” and words stemming therefrom encompasses inhibiting or otherwise blocking infection by ZIKV. As used herein, the term “inhibit” and words stemming therefrom may not be a 100% or complete inhibition or abrogation. Rather, there are varying degrees of inhibition of which one of ordinary skill in the art recognizes as having a potential benefit or therapeutic effect. In this respect, the presently disclosed expression vector systems or host cells may inhibit ZIKV infection to any amount or level. In illustrative embodiments, the inhibition provided by the methods of the present disclosure is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition).

In various embodiments, methods of the disclosure prevent, alleviate, and/or treat one or more symptoms associated with ZIKV infection. Illustrative symptoms that may be treated include, but are not limited to fever, rash (e.g., skin rash), muscle and/or joint pain, swollen joints, malaise, headache, conjunctivitis (red eyes), post-infection asthenia, digestive problems including abdominal pain, diarrhea, constipation, mucous membrane ulcerations (aphthae), pruritus, meningoencephalitis, and Guillain-Barré syndrome.

In various embodiments, methods of the present disclosure may prevent, alleviate, and/or treat one or more symptoms associated with ZIKV infection in pregnant women including those symptoms described above. Additionally, methods of the disclosure may prevent spontaneous abortions in pregnant women.

### Subjects

In exemplary embodiments, the subject referenced herein is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes).

In various embodiments, the mammal is a human. In some embodiments, the human is an adult aged 18 years or older. In some embodiments, the human is a child aged 17 years or less. In an embodiment, the subject is male, e.g., a male human. In another embodiment, the subject is a female subject. In illustrative embodiments, the subject is a female subject, e.g., a female human, aged from about 16 years to about 50 years. In illustrative embodiments, the female human is capable of giving birth. In illustrative embodiments, the subject is a pregnant female. In illustrative embodiments, the human pregnant female is in the first trimester, second trimester, or third trimester of pregnancy. In illustrative embodiments, the subject is not pregnant. In various embodiments, the subject is an embryo or a fetus including an unborn embryo or fetus. As referred to herein, an embryo is developed from the time of fertilization until the end of the eighth week of gestation, at which time it is referred to as a fetus. In exemplary aspects, the female human is pregnant or is considering whether or not to become pregnant.

### Samples

In exemplary embodiments, the sample referenced herein is a biological sample comprising one or more bodily fluids, e.g., human bodily fluids. In exemplary aspects, the sample comprises a bodily fluid, including, but not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In exemplary aspects, the sample is blood, plasma, serum, urine, semen, lacrimal fluid, saliva, or tissue fluids. In exemplary aspects, the sample comprises blood, plasma, serum, urine, cerebrospinal fluid, or saliva. In exemplary aspects, the sample comprises or is prepared from blood, plasma, or serum.

In exemplary aspects, the sample comprises or is prepared from blood, plasma, or serum and the sample further comprises one or more of: hemoglobin, bilirubin, cholesterol, rheumatoid factor, humanized anti-mouse antibodies (HAMA), and albumin. In exemplary instances, the sample comprises (i) hemoglobin or albumin at a concentration of at least about 75 mg/mL, about 125 mg/mL, or about 250 mg/mL, (ii) cholesterol at a concentration of at least about 2.5 mg/mL, about 5 mg/mL, or about 10 mg/mL, (iii) bilirubin or HAMA at a concentration of about 0.25 mg/mL, about 0.5 mg/mL, or about 1.0 mg/mL, (iv) or a combination thereof.

Alternatively or additionally, the sample comprises at least one infectious agent other than ZIKV. In some instances, the sample comprises one or more of: cytomegalovirus, Epstein-Barr virus, Parvovirus B19, varicella zoster virus, Plasmodium falciparum, chikungunya virus, Dengue virus, yellow fever virus, west nile virus, rheumatoid factor, Japanese encephalitis virus, St. Louis encephalitis virus, or antibody nuclear antibody (ANA).

The following examples are given merely to illustrate the present invention and not in any way to limit its scope.

### EXAMPLES

### Example 1

This example demonstrates the isolation of antibodies from human samples.

Zika virus (ZIKV) infection has become a serious public health concern with the potential to impact millions of individuals by the end of 2016. Of particular concern is the link between ZIKV infection of pregnant women and microcephaly, neurological impairment and distress in their offspring (Brasil et al., Zika Virus Infection in Pregnant Women in Rio de Janeiro—Preliminary Report, N Engl J Med (2016); e-pub ahead of print; Mlakar et al., N Engl J Med. 374(10):951-8 (2016)).

Human plasmablasts obtained from ZIKV-infected patients were sorted and enriched based on the selection of B cells that respond to replicating viral particles in a natural physiological context. Single cell cloning of the human plasmablasts was carried out as essentially described (Tiller et al. J Immunological Methods 329 (2008). Briefly, the heavy and light chains of antibodies were amplified using primers that align to the human V genes and expression vectors comprising a nucleotide sequence encoding the heavy and light chains were transfected into and expressed by human cells derived from the 293 cell line. Over 95 mAbs were expressed by four ZIKV-infected patients.

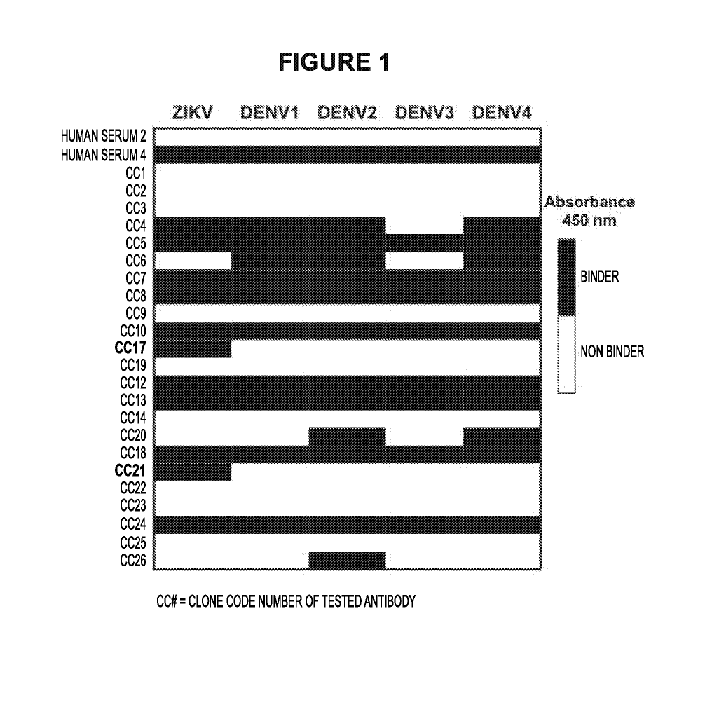

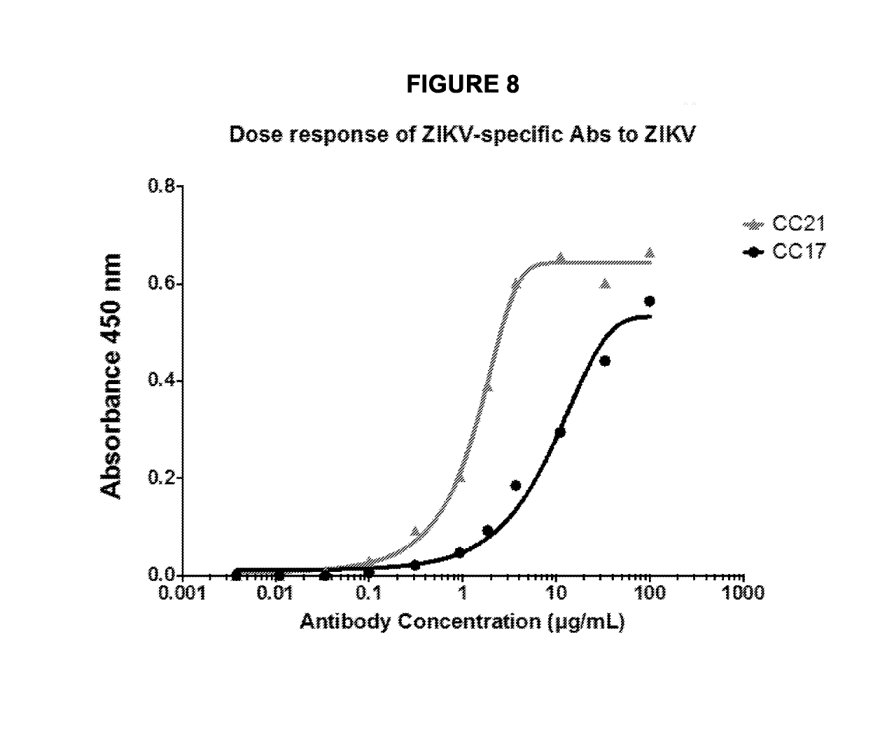

Binding assays of the mAbs were carried out as essentially described in Priyamvada et al. Proceedings of the National Academy of Sciences, 113 (2016). Of the >95 mAbs, twenty bound to ZIKV. However, only two mAbs were specific for ZIKV: CC17 and CC21—these two bound to ZIKV in a dose dependent manner (FIG. 8) and did not bind to any of the DENV subtypes 1-4 (FIG. 1).

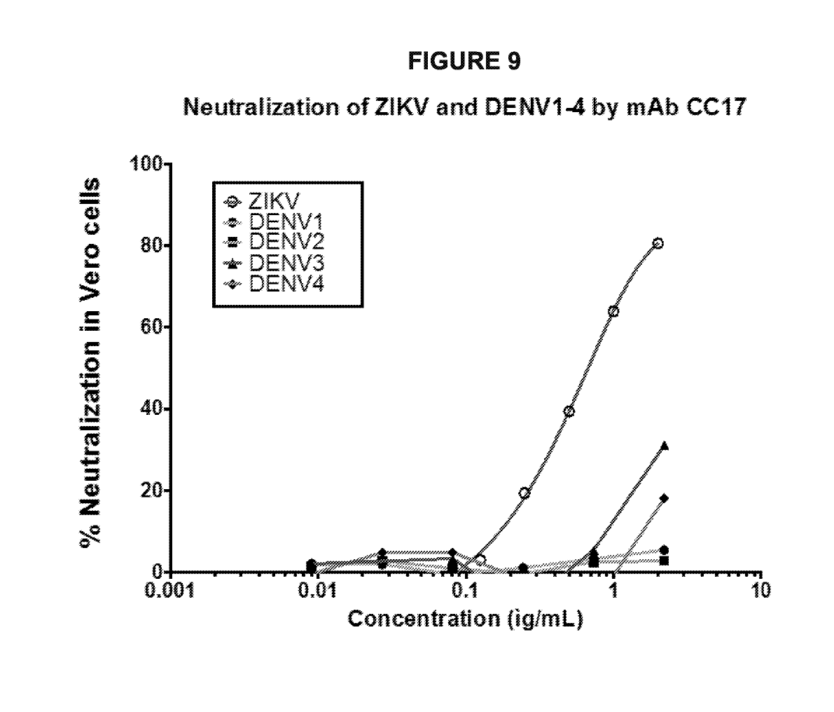

Select antibodies were tested for ZIKV neutralization in a Vero infectivity assay, as essentially described in de Alwis et al. Methods in Molecular Biology 11389 (2014) and Stettler et al. Science 353 (2016). Table 1 provides the results of the assay for several antibodies. The neutralization assay also tested for neutralization of the four DENV subtypes 1-4. It was found that the two ZIKV-specific mAbs (CC17 and CC21) did not bind to or neutralize any of the four DENV subtypes (FIG. 9).

TABLE 1

| | % of Zika virus | |
|---|---|---|
| Clone | Neutralized | Antibody |
| Code | (Vero infectivity | concentration |
| (CC) | assay) | (μg/ml) |
| 1 | 14.71 | 1 |
| 2 | 7.70 | 1 |
| 3 | 15.68 | 1 |
| 4 | 19.27 | 4 |
| 5 | 19.43 | 1.59 |
| 6 | 1.11 | 1.6 |
| 7 | 34.66 | 1.61 |
| 8 | 54.45 | 4 |
| 9 | 13.24 | 4 |
| 10 | 57.62 | 4 |
| 11 | −2.31 | 1.67 |
| 12 | 63.57 | 4 |
| 13 | 31.64 | 1 |
| 14 | 25.54 | 1.72 |
| 15 | 10.15 | 1.66 |
| 16 | 78.39 | 4 |
| 17 | 82.30 | 1 |
| 18 | 2.08 | 4 |
| 19 | 17.96 | 1.59 |

### Example 2

This example demonstrates a serological test for ZIKV exposure.

ZIKV-specific serum was used to inhibit binding of the CC17 mAb in a simple serological assay. First, ZIKV (specifically, a Brazilian ZIKV isolate from a patient in Fortaleza) was added to plates coated with the pan-flavivirus mAb 4G2 (MAB10216, clone D1-4G2-4-15; EMD Millipore, Darmstadt, Germany). ZIKV bound to plated 4G2 and sera from a variety of sources (21 sera samples in total) were pre-incubated with the bound ZIKV on the plates. The unlabeled CC17 mAb engineered to have rhesus monkey IgG1 constant regions was added to the plates. Binding of the CC17 mAb was detected using a horseradish peroxidase (HRP)-labeled mouse anti-rhesus mAb. An illustration of the assay is shown in FIG. 2.

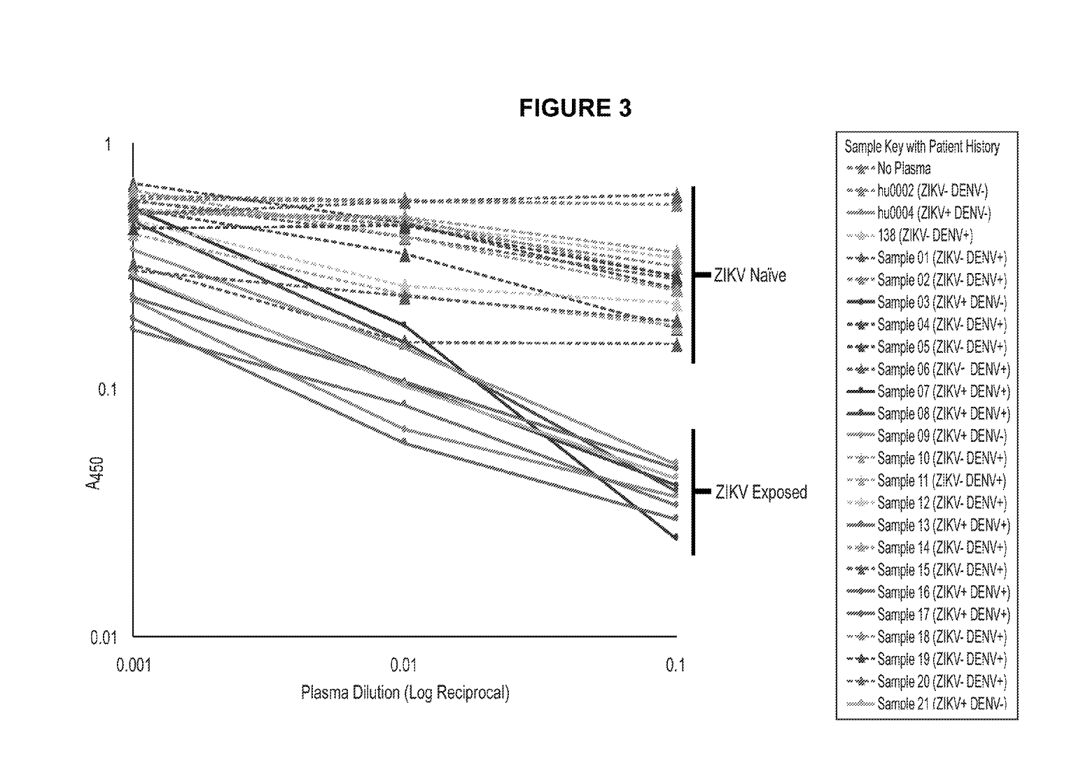

As shown in FIG. 3, only sera from individuals that had been previously exposed to ZIKV (designated by “ZIKV+”) blocked the binding of the highly specific CC17 mAb in a blinded test. Separately, a flow-based Plaque Reduction Neutralization Test (PRNT) of the 21 blinded sera samples was carried out and only the sera that inhibited ZIKV infection of Vero cells contained CC17mAb-blocking ability. These results suggest that a site recognized by CC17 mAb is also recognized by ZIKV-infected individuals in an immunodominant fashion.

### Example 3

This example demonstrates the design and development of two ZIKV diagnostic assays.

Rapid assays that are selective for direct detection of ZIKV or can easily detect prior exposure do not currently exist on the market. Current commercial assays detect ZIKV RNA (53), the presence of ZIKV-specific IgM (54) followed by the laborious Plaque Reduction Neutralization Test (PRNT) to detect previous exposure to ZIKV. The ultimate objective was to establish rapid clinical laboratory and PoC diagnostics for ZIKV.

The results in Examples 1 and 2 led to the goal of developing inexpensive, rapid (^{˜}20 min), laboratory-based and PoC diagnostics for previous ZIKV exposure. The diagnostics are to analyze both the acute and convalescent phases of ZIKV infection. Such tests are important in the family planning context. Women wishing to get pregnant will have access to effective and timely counseling based on ZIKV infection and prior exposure status. The results from the diagnostic assays can allow better control of spreading ZIKV to uninfected individuals.

Two different types of novel molecular diagnostic assays for the direct detection of ZIKV particles and prior exposure to ZIKV were planned. A first diagnostic assay planned for development is a microtiter-based immunoassay employing the newly developed mAbs was. The immunoassay is planned for use in clinical laboratories. A second diagnostic assay planned for development is a point of care (PoC) test based on a nitrocellulose membrane-based lateral flow assay. It is simple, low cost, portable and easy to use. It detects ZIKV infection and prior exposure to ZIKV with sensitivity and high selectivity. Because of the low complexity of this PoC assay, the expected cost of detection per patient sample is estimated to be about one-twentieth of the cost of currently available assays, and this PoC assay could be performed in clinical laboratories, doctor's offices or community health centers. The PoC diagnostic assay will not require instrumentation and will be operated with minimal operator expertise. Given its portability, the PoC assay can be used in remote locations, allowing access to healthcare to populations with unmet needs.

At the core of both diagnostic assays is the use of mAbs that are highly specific for ZIKV that will not bind to any of the DENV subtypes, such as those of the present disclosure. Another important point is that at least some of the antibodies of the present disclosure recognize an immunodominant epitope. For example, all tested ZIKV+ patients developed responses that interferes with this binding (FIG. 3). As opposed to a non-immunodominant epitope, which only a fraction of the patients would create responses against. The materials and methods described herein facilitate the rapid and specific diagnosis of ZIKV infection that is not currently available.

Design and Development of an Ab-Based Immunoassay Platform for Direct ZIKV Detection

To develop an Ab-based clinical laboratory-based and PoC, test, an assay that demonstrates that it can detect the virus and virus-specific Abs at the levels needed and in a direct manner is first devised. A microtiter plate-based ELISA for the direct detection of ZIKV was developed. FIG. 4 depicts the schematic of this diagnostic assay. As shown in this figure, plates coated with concanavalin A (ConA) capture ZIKV from samples on the plates. However, ConA can be replaced with the 4G2 antibody or the highly selective CC21 mAb described herein. Briefly, the wells of high-binding microtiter plates were coated with the lectin Con-A. Con-A binds specifically to the internal and non-reducing terminal α-mannose and glucose moieties found commonly in the glycosylated envelope proteins on the surface of many flaviviruses. Con-A has been used to capture viral particles for the development of various assay format ELISAs (58, 59). After coating the wells with Con-A, the sample containing ZIKV was added. The plate was then washed. After washing, detection Ab comprising rhesus monkey constant regions of the heavy and light chains was added to the plate. An anti-rhesus secondary antibody conjugated to horseradish peroxidase (HRP) was added and ZIKV was detected after the addition of the HRP substrate tetramethylbenzidine (TM B), which generates a colorimetric signal (FIG. 4).

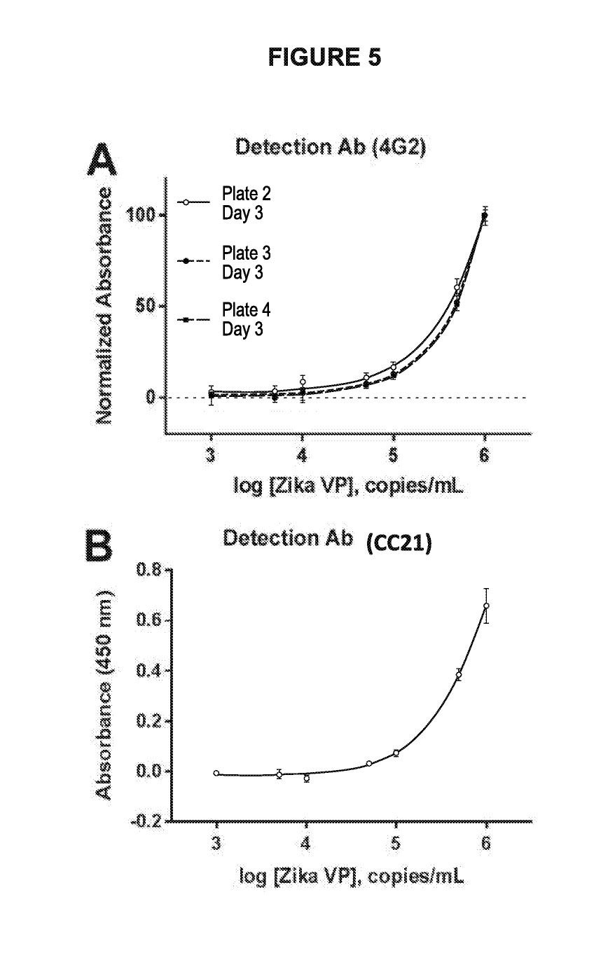

The results showed a dose-dependent response to ZIKV using the mAb 4G2 with a limit of detection of 10⁵ copies/mL. This assay was reproducible with a coefficient of variation of 7.2% (FIG. 5A), which is less than the accepted value for a diagnostic test by the US Food and Drug Administration (FDA). Using the ZIKV-specific CC21 mAb in place of 4G2 led to ZIKV detection in a specific and sensitive fashion (FIG. 5B).

ZIKV can be present in physiological fluids at levels as high as 1.0×10¹⁰ copies/mL and falls within the limit of detection of the assay using the current assay (21). The assay is currently carried out using CC17 mAb as a detection antibody and CC21 as a capture antibody to lower the detection limit. The developed platform may serve as a generic “plug and play” platform that can be used for the detection of ZIKV or ZIKV exposure with the CC17 and CC21 mAbs or any other mAb. The developed assay then serves as the template for miniaturization and development of the desired PoC test for ZIKV. Thus use of the highly specific mAbs described herein can be used for both diagnostics during the acute phase (to detect virus) and the convalescent phase (to detect the development of ZIKV-specific Abs).

Development of a Novel Laboratory-Based Diagnostic Approach for the Detection of ZIKV in Body Fluids in the Acute Phase and Serological Reactivity Against ZIKV in the Convalescent Phase

A simple, inexpensive method of specifically detecting ZIKV and prior exposure to ZIKV in individuals that may be experiencing symptoms or that need to either avoid or plan a pregnancy was designed. The method uses the CC17 and CC21 mAbs described herein.

The Ab-based assays for ZIKV (FIGS. 4 and 5) and ZIKV exposure (FIGS. 2 and 3) was adapted by using the highly specific mAbs CC17 and CC21 to directly bind to viral particles or be blocked by sera containing ZIKV-specific Abs.

A microtiter-based platform for the direct detection of ZIKV particles (FIG. 4) or prior exposure to the virus (FIG. 2) has been designed. The schematics depicted in FIGS. 2 and 4 show the components of the assay and the steps involved. The feasibility of developing a test for ZIKV and serological reactivity against ZIKV by employing the newly designed microtiter plate-based immunoassay platforms and simple mAb binding or mAb blocking assays has been demonstrated.

These platforms are optimized using a “checkerboard assay.” Concentrations of each antibody are varied against the antigen, as well as against each other, in order to find the concentration pairs at which the assay gives the best signal to noise ratio. These concentrations are then used throughout the remaining of the assay validation. In order to further improve the sensitivity and the detection limits of the assay, different types of substrates for HRP, including QuantaRed and luminol, that generate fluorescent or chemiluminescent signals, respectively, are employed. Moreover, other reporters including luciferase or other bioluminescent photoproteins known to achieve ultra-low detection limits, which have been used by us and others in immunoassays (60-66) are analyzed for employment in the diagnostic assay. To demonstrate the usefulness of these assays in the clinical diagnosis of ZIKV and prior exposure to ZIKV, the test is evaluated and validated using real clinical samples obtained from patients infected with ZIKV. A range of patient samples are run simultaneously with the currently developed test along with the current industry standard, RT-PCR based assays and PRNT assays. Thirty serum and urine samples (from both the acute phase and the convalescent phase—for a total of 60) from infected individuals and controls are tested and the values obtained by this method is compared against the current methods. The functional relationship between the two methods are then assessed using Deming or Passing-Boblock regression analysis (67). The agreement between this assay and the current gold-standard test, (PCR and PRNT-based), validate these assays and determine whether this assay can accurately detect active ZIKV and ZIKV exposure in infected individuals.

The novel laboratory immunoassay-based platforms described herein will not cross react with other common flaviviruses, and, hence can be used as a stand-alone clinical assay for the detection of active ZIKV infection and prior exposure. If needed, different blocking conditions and different dilutions of serum may be used. Also, as an alternative to Con-A coating, the plate wells may be coated using one of the ZIKV-specific mAbs described herein.

Design and Development of a PoC Test for Direct Detection of ZIKV and Prior Exposure to ZIKV

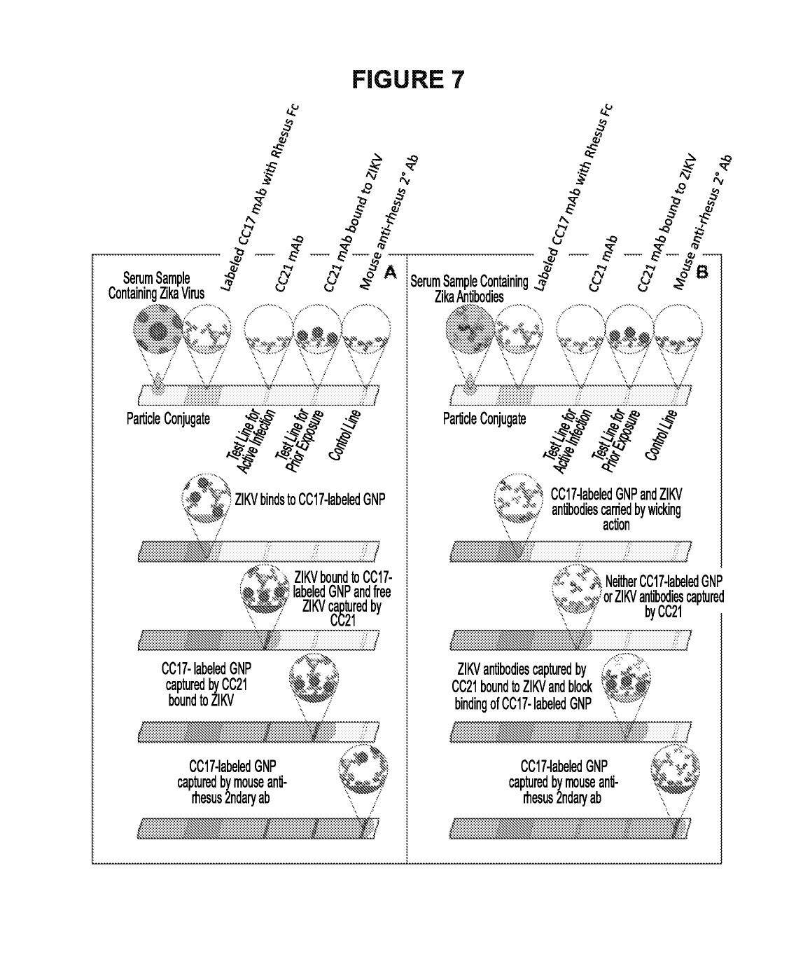

The microtiter plate platform developed above for clinical laboratory settings is a step forward in the direction toward better diagnostics for ZIKV. This has served as a stepping stone for the development of a PoC test based on the same principle. In that regard, a single PoC lateral flow assay (immunochromatographic assay) was developed to detect the presence of ZIKV and ZIKV exposure as depicted in FIG. 7A. Examples of reasons for employing the lateral flow principle include that it is (1) easy to use, (2) fast, (3) stable at different storage conditions, (4) portable, and (5) inexpensive. These characteristics make these types of assays ideally suited for home, PoC, and field tests in developed and developing countries, as well as in urban and/or rural settings, and even in remote locations.

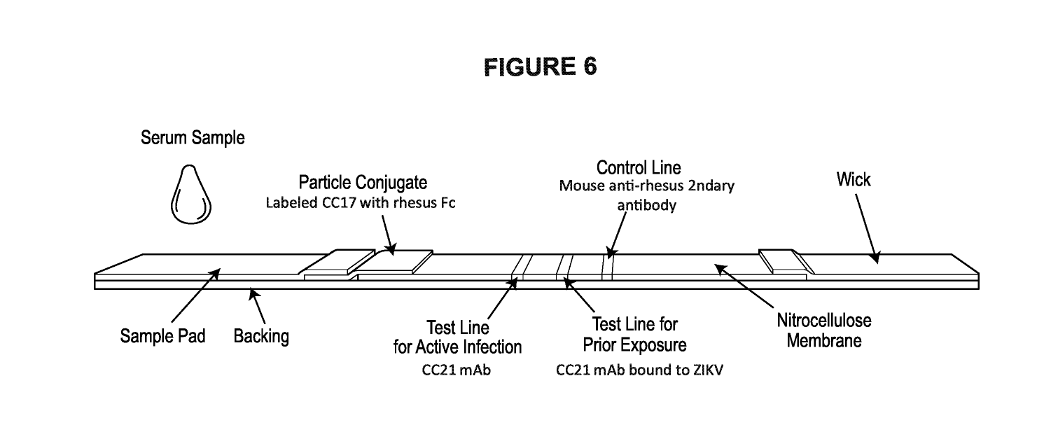

The lateral flow assay is prepared as follows: a rectangular sheet of nitrocellulose membrane is cut to the dimensions: 0.5 cm×5 cm. As mentioned, nitrocellulose is a very commonly used substrate for lateral flow assays and it is well established. Two test lines and a control line (FIG. 6) is laid down as thin strips using ink-jet printing technology (68). These zones contain CC21 mAb (zone A), CC21 mAb-bound ZIKV (zone B) and a mouse anti-rhesus IgG1 (zone C) (FIG. 6). The membrane is dried for 1 hour at RT and soaked with an aqueous solution of 1.0% polyvinyl alcohol (PVA) for 30 min at room temperature to make the nitrocellulose more hydrophobic and facilitate the flow of the reagents. The membrane is washed with deionized water to remove excess blocking reagents, such as PVA, and will be dried at 30° C. for 30 min. Then, the proximal end of the nitrocellulose strip, the region that contains the conjugated primary antibody against ZIKV, is prepared by applying a 30% solution of sucrose followed by baking for 1 hour at 40° C. Sucrose is typically used in lateral flow assays as a preservative and facilitates the long-term storage of the nanoparticle conjugated primary antibodies. It also has the advantage of being inert towards typical assay chemistries. This region (labeled as Particle Conjugate in FIG. 6) contains CC17 mAb conjugated to a nanoparticle such as gold or polystyrene. The collected patient samples are applied to the region labeled sample application pad (FIG. 6) at the proximal end of the test strip. The sample application pad is usually made of cellulose or glass fiber and the sample is applied onto this pad to start the assay. The sample may also be treated in this region to make it compatible with the rest of the test. The treatments may include removal of red blood cells from the serum, removal of interferences from the sample, adjustment of pH, etc. (69). The sample then migrates, through capillary action, through the nitrocellulose strip to the Particle Conjugate region. If the sample contains ZIKV, the dried primary Ab conjugated to the nanoparticles are remobilized and the ZIKV particles bind to these conjugated primary antibodies. The formed complexes flow through the reaction matrix, which is usually a porous matrix such as nitrocellulose. This matrix also contains the other biological components of the assay, again laid down as thin, narrow bands using ink jet printing. In the first band (zone A) the mAb CC21 is immobilized. The labeled ZIKV is captured by the immobilized CC21 forming a colored band (FIG. 7). The second band (zone B) will contain CC21-ZIKV complexes. If the sample contains Abs against ZIKV, indicating prior infection, the Abs will inhibit binding of the CC17 conjugated to nanoparticles and will be negative (FIG. 7). Immobilized anti-Rhesus secondary antibodies are present in the third zone and serve as a positive control for both tests.

The results are interpreted as the presence and absence of lines of the captured conjugate that can be read either by eye or by using a hand-held, battery operated, smart-phone based absorbance, fluorescence or luminescence reader. The type of the reader depends on the type of the labels conjugated to the primary antibodies. Therefore, the presence of two bands (zones A and B) indicate ZIKV viremia, whereas the presence of a band in only zone B indicates previous exposure. A single band in the control zone C indicates a negative result for an active ZIKV infection and prior exposure. The absence of a colored band in either of the test zones (Zones A and B) indicate a failed test. The test will then be validated using real patient samples which was measured previously using our microtiter assay. Since this lateral flow test is a self-contained, self-reporting device, the need for elaborate laboratory equipment will be eliminated.

The assay described above is optimized and suitable for use for the serological detection of ZIKV and ZIKV exposure in urban, rural, and low resource settings. The PoC device is selective for ZIKV and does not cross react with other common flaviviruses. Therefore, this assay can be used in low-resource settings where access to modern laboratory instrumentation is limited. If needed, sensitivity may be increased by 1 to 2 orders of magnitude by employing a fluorescent label in conjunction with a dedicated hand-held, battery operated, fluorescence reader or a digital camera or a smart phone that can image the color generated.

### Example 4

This example demonstrates monoclonal antibodies for the selective detection of Zika virus (ZIKV).

Zika virus is a member of the flavivirus genus, which contains members sharing a high degree of sequence similarity. This means an antibody generated against one member of the genus is likely to cross-react with other members. The Zika and Dengue viruses are not only extremely similar but frequently concurrent. Due to the confirmed coincidence of microcephaly, fetal damage, and Guillain-Barré syndrome in infants born to Zika-infected mothers, there is an urgent need for the selective and specific detection of exposure to this virus in point-of-care and laboratory settings to facilitate family planning. Since only 20% of Zika-infected individuals develop symptoms, most women planning a pregnancy in a Zika endemic area will want to know whether they have been previously infected. Prior infection will likely induce protective immunity and re-infection will be difficult.

Six highly-specific monoclonal antibodies against Zika virus have been identified and are readily adaptable for use in laboratory or point-of-care testing. These monoclonals, do not bind to any of the closely-related Dengue virus serotypes. This is important due to the potential for co-infection of the most closely-related Zika and Dengue viruses in many regions.

Further, a sandwich ELISA serological test for Zika using one of the monoclonals (CC17), has the potential to replace both IgM and plaque reduction neutralization tests in the post-acute phase (right). The blinded test results (below) highlight specificity. CC17 is suitable to develop an inexpensive, rapid (20 minute), diagnostic for prior Zika virus exposure in both laboratory and point-of-care settings. Because CC17 recognizes a neutralizing epitope, it may also be useful in assessing vaccine efficiency.

This example demonstrated that six monoclonal antibodies have been isolated and characterized for the detection of ZIKV infection. These antibodies have been used in the diagnosis of Zika virus during the convalescent phase of infection.

### Example 5

This example demonstrates the development of an antibody-based clinical laboratory assay which can detect in a direct manner ZIKV and ZIKV-specific antibodies at the levels required for detection in physiological fluids.

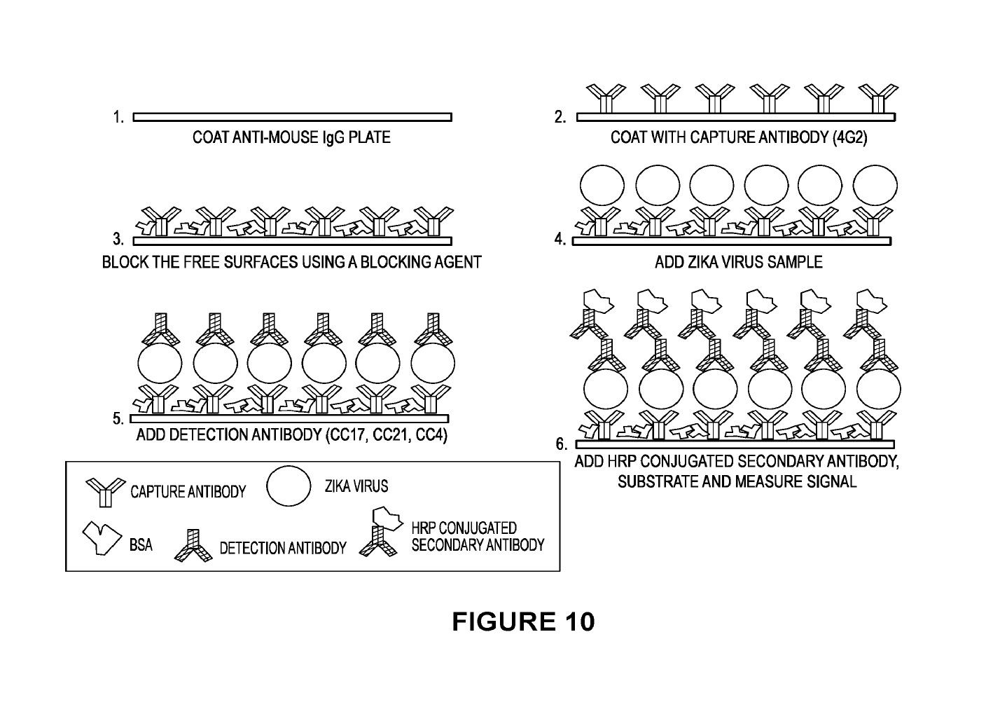

The assay was designed as a microtiter plate-based immunoassay for direct detection of ZIKV, wherein, initially, the commercially available 4G2 antibody against ZIKV was used. The assay has been modified to utilize the highly selective monoclonal antibodies (mAbs) CC17, CC21 and CC4 described herein. The assay was first designed on an ELISA platform for the direct detection of the ZIKV. The schematic of the ELISA, as well as the components of the assay and the steps of the assay, are depicted in FIG. 10. For this assay, the assay plates were coated with the 4G2 antibody that recognizes a variety of flaviviruses and captures the virus on the plates. Briefly, the wells of high-binding microtiter plates were coated with 4G2 antibody in 100 mM bicarbonate buffer at pH 9.60 overnight at 4° C. and it was used to capture viral particles for the development of our ELISAs.

After the coating step, the plates were washed using PBST (10 mM sodium phosphate dibasic, 2 mM potassium phosphate monobasic, 137 mM sodium chloride, 2.7 mM potassium chloride, 0.05% Tween 20, pH 7.40) and the wells were blocked using 200 μL of commercially available blocking buffer (Starting Block PBS from Thermo Fisher). The wells were washed using PBST after the blocking step. Culture samples containing different concentrations of ZIKV, ranging from 1×10³ copies/mL to 1.0×10⁶ copies/mL, were added to the wells in triplicate. The wells were then washed. Subsequently, mAbs of the present disclosure (CC17, CC21 and CC4) having rhesus monkey constant regions diluted at different concentrations in Zika Incubation buffer (PBSTB: 10 mM sodium phosphate dibasic, 2 mM potassium phosphate monobasic, 137 mM sodium chloride, 2.7 mM potassium chloride, 0.05% Tween 20 and 1.0% BSA, pH 7.40) were added to the wells of the plate. After an incubation time, the wells were washed with PBST, and 100 μL aliquots of an anti-rhesus secondary antibody conjugated to horseradish peroxidase (HRP) diluted to 62.5 ng/mL in Zika incubation buffer was added. Finally, ZIKV was detected after the addition of the HRP substrate tetramethylbenzidine (TMB), which generated a colorimetric signal (FIG. 10).

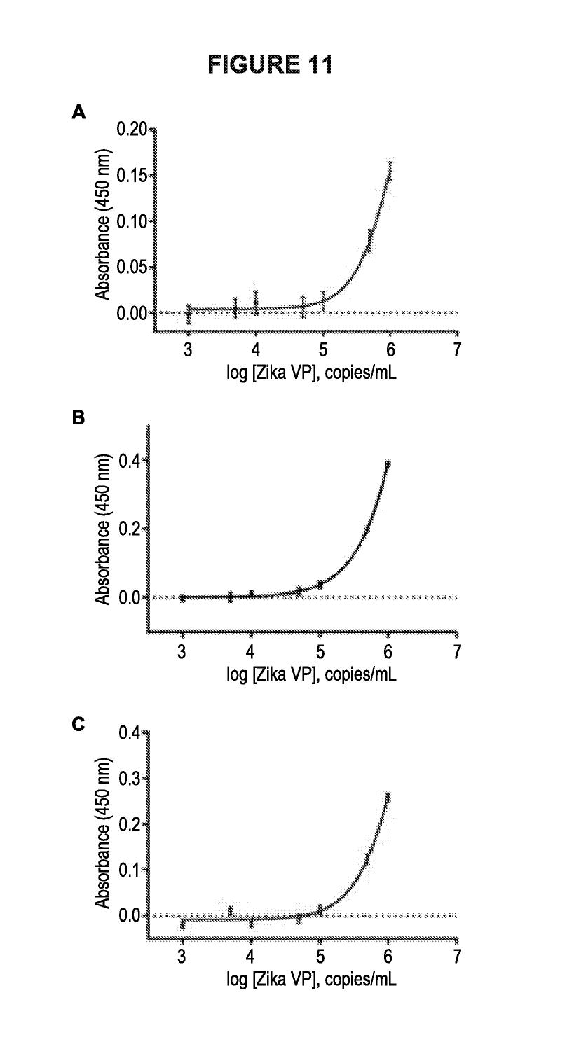

As shown in FIGS. 11A-C, each mAb exhibited a dose-dependent response to ZIKV with a limit of detection of 10⁵ copies/mL. This detection limit is important as ZIKV is present in physiological fluids at levels as high as 7.0×10⁵ copies/mL, which falls within the limit of detection of this assay.

The assay is being developed to lower the detection limit even further. It is important to note that the developed platform serves as a generic “plug and play” platform that can be used for the detection of ZIKV or ZIKV exposure with the CC17, CC21, and CC4 mAbs or any other mAb that is described herein. The developed assay serves as the template for miniaturization and development of the desired point-of-care (PoC) test for ZIKV. Thus the highly specific mAbs described herein can be used for diagnostics during the acute phase to detect virus.

### Example 6

This example demonstrates a checkerboard assay of the assay described in Example 5.

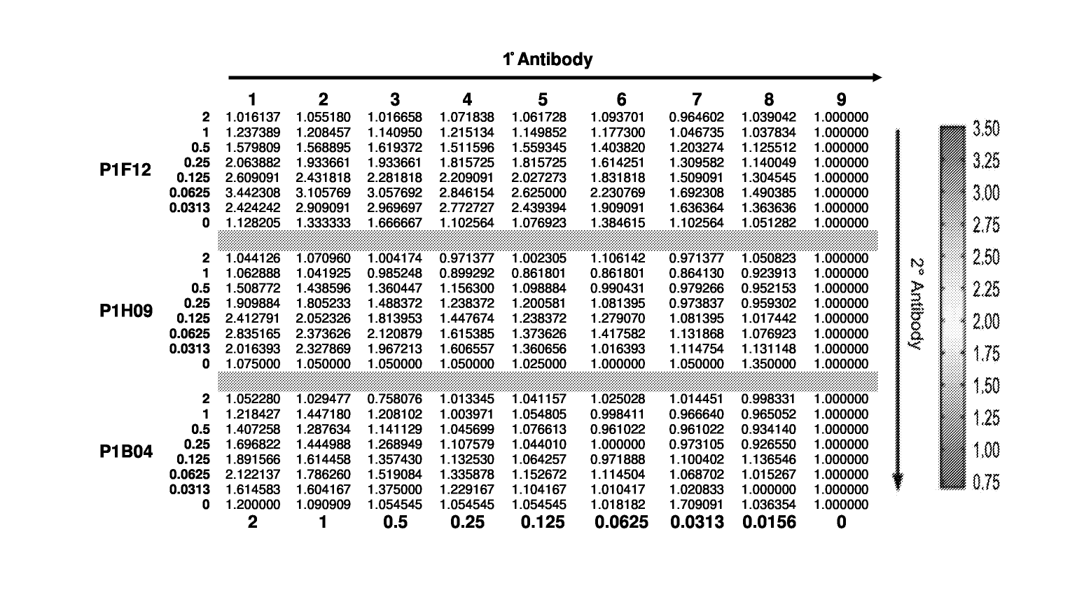

In the checkerboard assay, concentrations of each antibody were varied against a constant concentration of the antigen, as well as against each other, in order to find the concentration pairs at which the assay gives the best signal to noise ratio (FIG. 12). Briefly, the concentration of the ZIKV were kept at 1.0×10⁶ copies/mL while the concentrations of the primary antibodies and the secondary antibody were varied between 15.6 ng/mL to 2.0 μg/m L. From this experiment, optimum primary and secondary antibody concentrations were calculated. The optimum concentrations for the primary antibodies were estimated to be 0.125, 0.5 and 1.0 μg/mL for CC4, CC17 and CC21, respectively. The optimum concentration for the secondary antibody was estimated to be 62.5 ng/mL. These concentrations were used throughout the remaining of the assay optimization and validation.

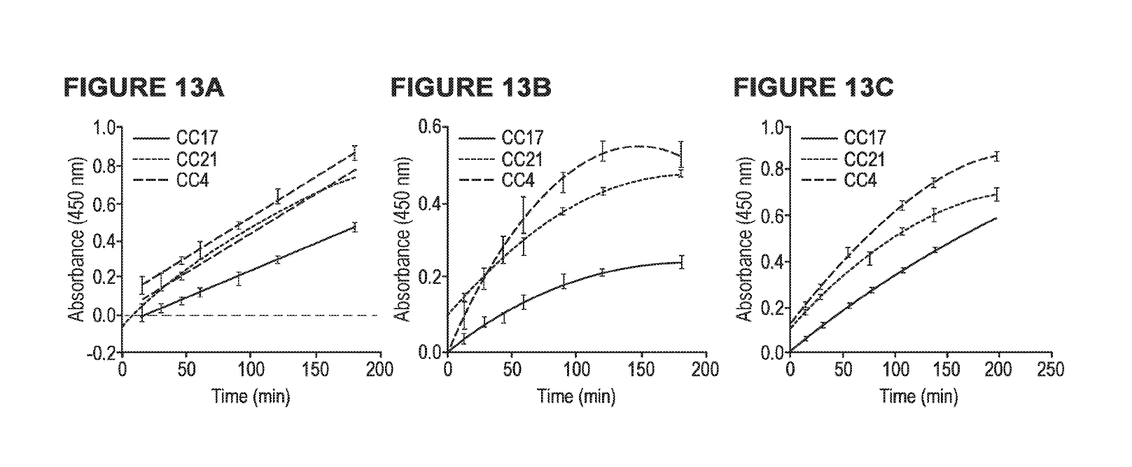

Next, the incubation times were analyzed in order to maximize the signal-to-noise ratio. (FIGS. 13A-C). The time points were used to validate our assay with respect to its sensitivity and selectivity, as well as for its precision and accuracy.

Different types of substrates for HRP, including QuantaRed and luminol, that generate fluorescent or chemiluminescent signals, respectively, are evaluated. The same assay is also adapted to other types of reporters such as luciferase or other bioluminescent photoproteins known to achieve ultra-low detection limits, which have been used in immunoassays.

### Example 7

This example demonstrates the development of a ZIKV point of care (POC) test for direct detection of active exposure to ZIKV.

The microtiter plate platform developed above in Examples 5 and 6 for clinical laboratory settings is a step forward in the direction toward better diagnostics for ZIKV. This assay platform serves as a stepping stone for the development of a PoC test that will be based on the same principle as our microtiter plate clinical laboratory assay. In that regard, we designed and developed a PoC lateral flow assay (immunochromatographic assay) to detect the presence of ZIKV (FIG. 7A). The reason for employing the lateral flow principle is because it is (1) easy to use, (2) fast, (3) stable at different storage conditions, (4) portable, and (5) inexpensive. These characteristics make these types of assays ideally suited for home, PoC, and field tests in developed and developing countries, as well as in urban and/or rural settings, and even in remote locations. It was decided to design a PoC test by miniaturizing and adapting our developed clinical laboratory-based technology employing lateral flow principles.

The lateral flow assay is prepared as follows: a rectangular sheet of nitrocellulose membrane is cut to the required dimensions (0.5 cm×5 cm). Nitrocellulose is a commonly used substrate for lateral flow assays and it is well established. Then, two test lines and a control line (FIG. 6) are laid down as thin strips using ink-jet printing technology. These zones contain CC21 (zone A), CC21-bound ZIKV (zone B) and a mouse anti-rhesus IgG1 (zone C) (FIG. 6). The membrane is then be dried for 1 hour at RT and soaked with an aqueous solution of 1.0% polyvinyl alcohol (PVA) for 30 min at room temperature to make the nitrocellulose more hydrophobic and facilitate the flow of the reagents. The membrane is then be washed with deionized water to remove excess blocking reagents, such as PVA, and is dried at 30 [symbol] C for 30 min. Then, the proximal end of the nitrocellulose strip, the region that contains the conjugated primary antibody against ZIKV, is prepared by applying a 30% solution of sucrose followed by baking for 1 hour at 40° C. Sucrose is typically used in lateral flow assays as a preservative and facilitates the long-term storage of the nanoparticle conjugated primary antibodies. It also has the advantage of being inert towards typical assay chemistries. This region, (labeled as Particle Conjugate in FIG. 6) contains the primary mAb CC17 conjugated to a nanoparticle such as gold or polystyrene. The collected patient samples will be applied to sample application pad at the proximal end of the test strip. In the sample application pad is usually made of cellulose or glass fiber and the sample is applied onto this pad to start the assay. The sample may also be treated in this region to make it compatible with the rest of the test. The treatments may include removal of red blood cells from the serum, removal of interferences from the sample, adjustment of pH, etc. (69). The sample then migrates, through capillary action, through the nitrocellulose strip to the Particle Conjugate region. If the sample contains ZIKV, the dried primary Ab conjugated to the nanoparticles are remobilized and the ZIKV particles bind to these conjugated primary antibodies. The formed complexes flow through the reaction matrix, which is usually a porous matrix such as nitrocellulose. This matrix also contains the other biological components of the assay, again laid down as thin, narrow bands using ink jet printing. In the first band (zone A) the mAb CC21 will be immobilized. The labeled ZIKV is then captured by the immobilized CC21 forming a colored band (FIG. 7). Immobilized anti-Rhesus secondary antibodies present in the third zone serve as a positive control for both tests. The results are interpreted as the presence and absence of lines of the captured conjugate that can be read either by eye or by using a hand-held, battery operated, smart-phone based absorbance, fluorescence or luminescence reader. The type of the reader depends on the type of the labels conjugated to the primary antibodies. Therefore, the presence of two bands (zone A and B) indicates ZIKV viremia. A single band in the control zone C indicates a negative result for an active ZIKV infection. The absence of a colored band in either of the test indicates a failed test. Since this lateral flow test is a self-contained, self-reporting device, the need for elaborate laboratory equipment is eliminated.

### Example 8

This example demonstrates a human inferred germline antibody binds to an immunodominant epitope and neutralizes Zika virus.

The isolation of neutralizing monoclonal antibodies (nmAbs) against the Zika virus (ZIKV) might lead to novel preventative strategies for infections in at-risk individuals, primarily pregnant women. Here we describe the characterization of human mAbs from the plasmablasts of an acutely infected patient. One of the 18 mAbs had the unusual feature of binding to and neutralizing ZIKV despite not appearing to have been diversified by affinity maturation. This mAb neutralized ZIKV (Neut50 ^{˜}2 μg/ml) but did not react with any of the four dengue virus serotypes. Except for the expected junctional diversity created by the joining of the V-(D)-J genes, there was no deviation from immunoglobulin germline genes. This is a rare example of a human mAb with neutralizing activity in the absence of detectable somatic hypermutation. Importantly, binding of this mAb to ZIKV was specifically inhibited by human plasma from ZIKV-exposed individuals, suggesting that it may be of value in a diagnostic setting.

Antibody affinity maturation through somatic hypermutation (SHM) is thought to be critical for the development of antibodies with virus-neutralizing activity. Contrary to this notion, we describe novel human anti-Zika virus (ZIKV) antibodies with very low mutation levels, isolated from plasmablasts early after the onset of symptoms. Surprisingly, one IgG monoclonal antibody, P1F12, bound to ZIKV and neutralized the virus, despite having no detectable mutations. This antibody is specific for ZIKV and did not cross-react with DENV. Furthermore, plasma from ZIKV-positive individuals blocked the interaction of P1F12 with ZIKV, whereas plasma from DENV-positive patients did not have this inhibitory ability. P1F12 targets an immunodominant site, as ZIKV-positive samples blocked P1F12-ZIKV binding. Our study shows that isotype-switched virus-specific neutralizing Abs can develop in humans directly from germline sequences.

Zika virus (ZIKV) belongs to the genus Flavivirus of the Flaviviridae family and is related to dengue virus (DENV), yellow fever virus (YFV), Japanese encephalitis virus (JEV), and west Nile virus (WNV)¹. The globally distributed mosquito species of the Aedes genus, vectors for many Flavivirus, can also transmit ZIKV^{2,3}. However, ZIKV remained a relatively minor and obscure cause of human disease for most of the second half of the 20^{th} century and was featured in a limited number of scientific reports. In fact, it was not until 2007 that autochthonous human infection was described outside Africa and continental Asia—in the Federated States of Micronesia⁴⁻⁶. Since then, reports from Brazil have chronicled a rapidly spreading epidemic that co-exists with DENV and chikungunya virus (CHIKV). The epidemic has spread north with mosquito-borne transmission being reported in many nations of the Americas as far north as Mexico and southern Florida⁷⁻⁹. More ominously, ZIKV has been implicated as the causative agent in fetal developmental problems^{10,11}. There are reports of ZIKV-associated birth defects, primarily brain abnormalities and microcephaly in infants born to mothers infected with ZIKV¹². Virus has been recovered from amniotic fluid, placental, and brain tissues¹³⁻²¹. ZIKV infection has been classified as an ongoing threat by the World Health Organization. In the United States, the Centers for Disease Control and Prevention has issued guidance for the management of the infection in the general population, pregnant women, and infants²²⁻²⁴. Due to recent reports of sexually transmitted ZIKV infection, the CDC has also developed guidelines for prevention of this mode of transmission²²⁻²⁷. More recently, ZIKV transmission has also been described in Miami, Fla.²⁸, suggesting that autochthonous spread could occur in any region of the U.S. inhabited by Aedes spp.

Treatment of a variety of human ailments using mAbs is revolutionizing our ability to ameliorate human suffering. For infectious disease, the Ebola epidemic highlighted the potential utility of a cocktail of three neutralizing (n)mAbs that block infection by the Ebola virus²⁹. Most convincingly, the administration of a single nmAb up to five days post infectious virus exposure prevents the development of disease in Ebola-infected macaques³⁰. Because mAbs can be engineered to prevent antibody-dependent enhancement by incorporating the L234A and L235A (LALA) mutations which reduce FcγR binding³¹, they are a promising intervention in flaviviral therapies. Our long-term goal is to use a cocktail of LALA-mutated nmAbs to prevent ZIKV infection in at-risk individuals, primarily pregnant women.

Therapeutic nmAbs must be potent in order to be clinically viable, and most nmAb isolation strategies are based on the identification of high-titer, antigen-selected repertoires. Somatic hypermutation (SHM) in germinal center (GC) B cells provides the basis for selection of B cells producing Abs with increased affinity—a hallmark of the adaptive humoral response. This feature is conserved among mammals, highlighting the importance of Ab affinity enhancement for evolutionary fitness³². Thus, it is unsurprising that the vast majority of human Abs in the memory immunoglobulin (Ig)G pool have undergone affinity maturation and have, on average, 10-26 nucleotide substitutions from precursor genes³³. The contribution of SHM to Ab-mediated viral neutralization is particularly clear for the chronically-induced broadly neutralizing antibodies to HIV³⁴⁻³⁷. Reversion of these anti-HIV nmAbs to precursor germline antibodies results in a drastic reduction or complete loss of viral neutralization³⁸⁻⁴¹. Although mutated mAbs are found after secondary DENV infection, the role of these mutations in acute virus-neutralization and clearance is less clear⁴²⁻⁴⁵. Still, the prevalent thought is that antiviral Ab response involves the engagement of poor- or non-neutralizing germline clones generated by V(D)J rearrangement, followed by SHM-mediated refinement in germinal centers to enhance neutralization potency.

Here we describe the isolation of 18 plasmablast-derived human mAbs, sorted 12 days post onset of symptoms from a ZIKV-patient in São Paulo, Brazil. The patient reported a previous history of dengue infection and yellow fever vaccination (TABLE 2). A few of the isolated Abs neutralized ZIKV, most of them at relatively high concentrations. Interestingly, one of these mAbs (P1F12, also referred to herein as CC17) exhibited no nucleotide mutations when compared to its corresponding germline sequences, but still recognized a ZIKV immunodominant epitope and neutralized the virus. These results suggest unforeseen roles for GC-independent responses against ZIKV and possibly other viruses.

TABLE 2
Patient Details

| | | | | | | | Initial | | Time of |
|---|---|---|---|---|---|---|---|---|---|
| Identification | | | | Diagnosis | | Medical | Symptoms | Clinical | Plasmablast |
| ID | City | Sex | Age | Urine | Blood | history | (D 0) | history^{a} | Sort^{a} |
| 533 | São | F | 56 | PCR | PCR | dengue | Rash, | GBS initiation (D 6), | D 12 |
| | Paulo | | | positive | negative | fever, YF | Myalgia, | Hospitalized (D 10), | |
| | | | | | | vaccinated | Arthralgia | IVIG treated (D 12). | |

^{a}Time point after onset of symptoms.

Results

Patient

We isolated plasmablasts from patient 533 who presented with suspected Guillain-Barré syndrome (GBS) (TABLE 2) (first day of symptoms=D0). The patient had a previous history of dengue infection and yellow fever vaccination (Table 2). The previously healthy 56-year-old woman presented to the emergency room (D6) reporting a progressive paresthesia mainly in the extremities of her hands, along with acute, intermittent pain in her left forearm during the previous four days. At physical examination, the patient presented with a grade IV asymmetric muscular weakness and hypoesthesia in her left limbs, with abolished deep tendon reflexes in the lower limbs. A mild weakness of her left facial muscles was also noted. The patient reported no respiratory disorders and no hoarseness, and no signs of dysautonomia were detected at the clinical evaluation. Fever, conjunctivitis, and myalgia or joint pain were absent during the illness. Afterwards, the patient was hospitalized with a clinical diagnosis of GBS, for which an intravenous human Ig (IVIG) treatment was initiated at a dosage of 0.4 g/kg/day for 5 days. Cerebrospinal fluid analysis and an electroneuromyogram were performed on fourth (D10) and fifth (D11) days after neurological symptom onset, respectively; the results were within normal limits. The electroneuromyogram was repeated on the 15^{th} day of neurological symptoms, but no significant abnormalities were noted despite the persisting weakness in the patient's left leg and arm. During the treatment with IVIG, the patient presented with transient worsening of her hemiparesis, but progressively recovered over the course of weeks after discharge from the hospital. At 32 days post-neurological symptom onset (D38), a physical exam revealed significant improvement of muscular strength and abolished deep tendon reflexes in the lower limbs. The remittent skin rash cleared completely 10 days after its initial emergence.

Blood, cerebrospinal fluid and urine samples were collected on the 5^{th} day of neurological symptoms (D11) for detection of ZIKV by RT-PCR. The urine sample was ZIKV-positive by PCR, while blood and cerebrospinal fluid were negative. A saliva sample collected on D15 was negative for ZIKV.

Isolation, Binding, and Neutralization Testing of mAbs

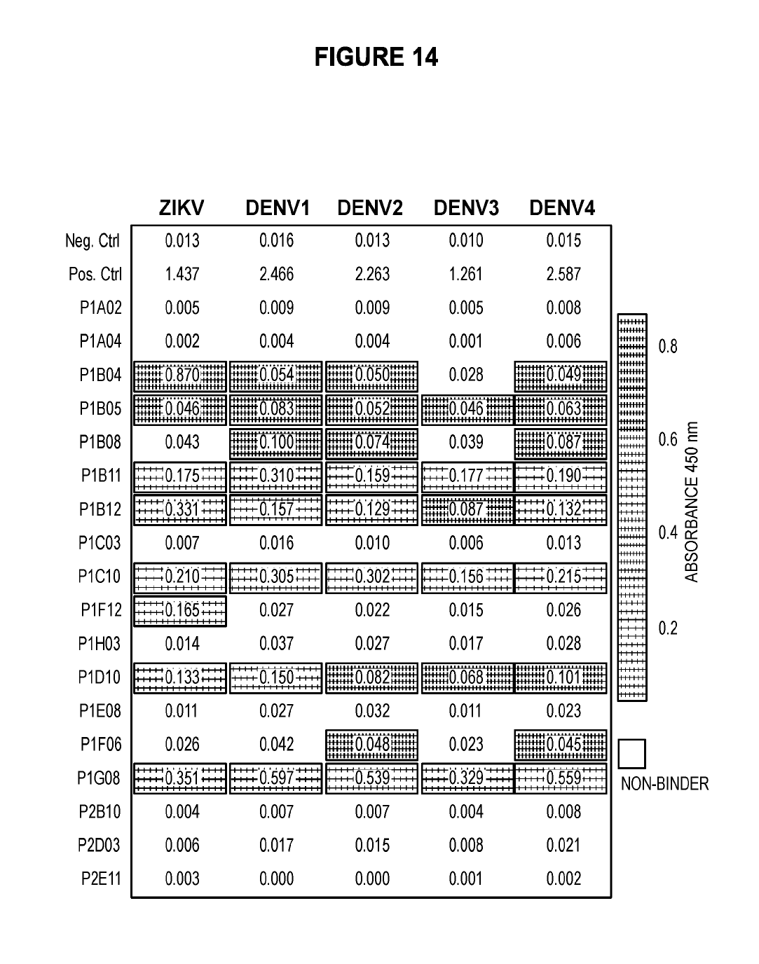

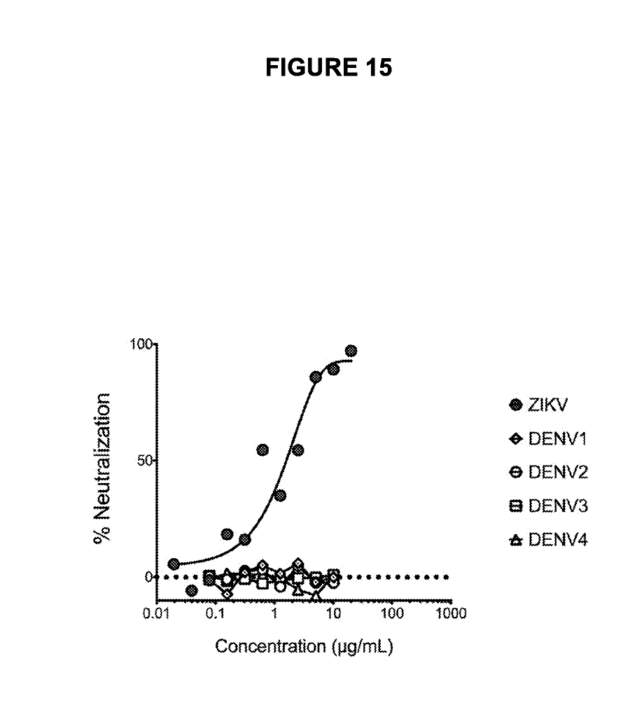

We isolated plasmablasts from peripheral blood mononuclear cells (PBMCs) collected on day 12 (TABLE 2). From wells containing single-sorted cells, we amplified, cloned, and sequenced heavy and light Ab chains using 5′ primers complementary to the V gene segments and a 3′ primer annealing to the constant IgG region⁴⁶. This resulted in 18 paired heavy and light chains (TABLE 3). Eight of the 18 mAbs bound to ZIKV (FIG. 14). Seven of these mAbs exhibited cross-reactivity to one or more of the DENV serotypes, and a single mAb-P1F12-bound exclusively to ZIKV. Interestingly, two mAbs bound to DENV but not ZIKV. We tested the neutralization potency of the ZIKV-specific P1F12 mAb in a flow-based neutralization assay and a plaque reduction neutralization test (PRNT) and found that it neutralized ZIKV at approximately 2 μg/ml (PRNT₅₀) (FIG. 15).

TABLE 3
Gene Usage and SHM levels in the plasmablast-derived mAbs

| | ZIKV | Human | Divergence | | Human | Divergence | |
|---|---|---|---|---|---|---|---|
| | binding | heavy chain | (aa) from | SEQ ID # | light chain | (aa) from | SEQ ID # |
| CC # | Abs 450 nm | V gene IGH . . . | germline | of CDRH3 | V gene IG | germline | of CDRL3 |
| 1 | 0.01 | V5-51*01 | 4 | 70 | KV1-39*01 | 7 | 71 |
| 2 | 0.00 | V1-18*01 | 10 | 72 | KV1-27*01 | 5 | 73 |
| 4 | 0.87 | V4-4*07 | 1 | 74 | KV3-11*01 | 1 | 75 |
| 5 | 0.05 | V3-23*04 | 1 | 76 | KV1-39*01 | 1 | 77 |
| 6 | 0.04 | V4-39*07 | 21 | 78 | KV3-20*01 | 14 | 79 |
| 7 | 0.18 | V3-23*04 | 1 | 80 | KV1-39*01 | 1 | 81 |
| 8 | 0.33 | V4-34*01 | 0 | 82 | KV3-20*01 | 0 | 83 |
| 9 | 0.01 | V4-4*07 | 19 | 84 | KV3-15*01 | 11 | 85 |
| 10 | 0.21 | V3-23*04 | 0 | 86 | KV1-39*01 | 0 | 87 |
| 17 | 0.17 | V3-7*01 | 0 | 88 | KV1-8*01 | 0 | 89 |
| 19 | 0.01 | V4-59*08 | 12 | 90 | KV4-1*01 | 6 | 91 |
| 12 | 0.13 | V3-21*01 | 1 | 92 | LV6-57*02 | 3 | 93 |
| 14 | 0.01 | V4-39*07 | 19 | 94 | LV3-1*01 | 20 | 95 |
| 15 | 0.03 | V3-21*01 | 0 | 96 | LV1-44*01 | 0 | 97 |
| 18 | 0.35 | V4-34*01 | 3 | 98 | LV1-51*01 | 0 | 99 |
| 22 | 0.00 | V3-23*04 | 9 | 100 | KV1-16*02 | 7 | 101 |
| 23 | 0.01 | V3-23*04 | 17 | 102 | KV1-5*03 | 8 | 103 |
| 25 | 0.00 | V3-23*04 | 2 | 104 | KV3-11*01 | 0 | 105 |

Unusual Sequence of P1F12

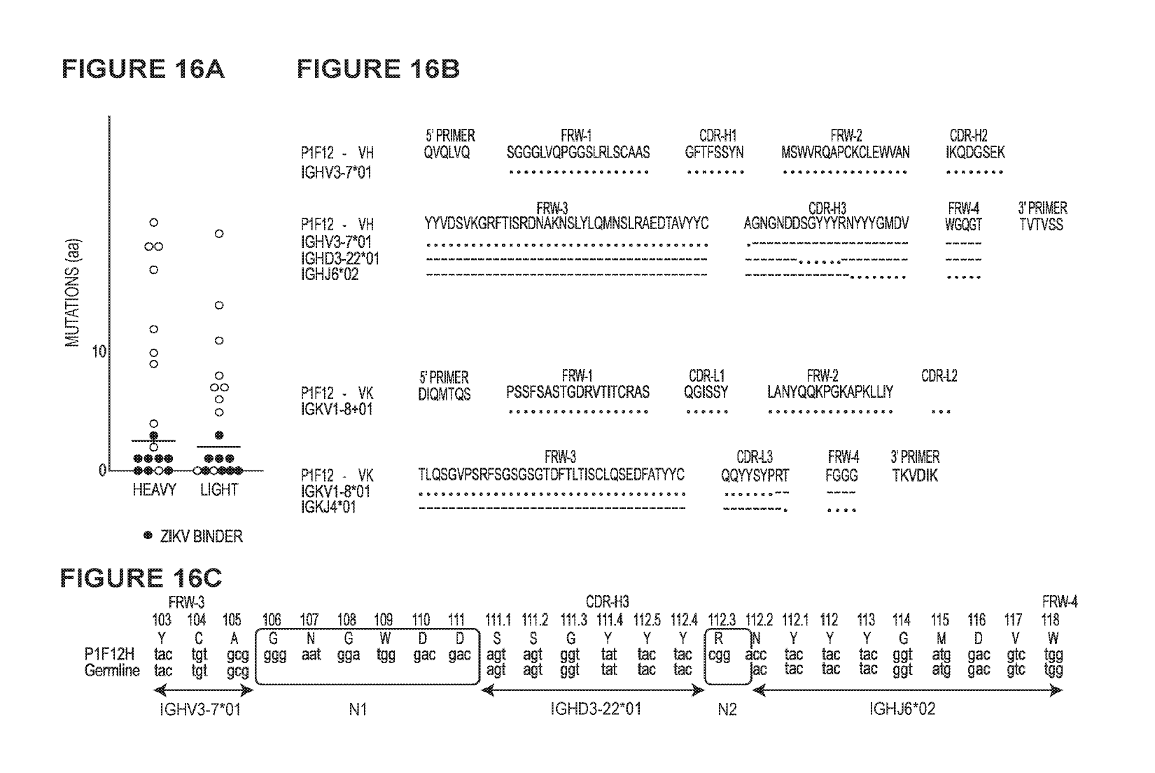

Analysis of the isolated antibody variable (V) domain sequences revealed five mAbs with average gene mutation levels (10-26 nucleotide modifications), two mAbs with over 30 nucleotide substitutions, and 11 mAbs with unusually low levels of SHM for isotype-switched mAbs (lower than 10 changes) (TABLE 3). The most highly mutated mAbs (P1B08 and P1C03, also referred to herein as CC6 and CC9) were not ZIKV-specific by binding (TABLE 3). In fact, the eight ZIKV-binding mAbs had the lowest SHM levels, including four mAbs lacking clearly recognizable mutations when compared with putative heavy and light chain germline precursors (TABLE 3, FIG. 16). Except for junctional diversity, the ZIKV-neutralizer P1F12 mAb heavy chain did not exhibit signs of antigen-selected Ig diversification. P1F12 had an identical sequence to the Ig heavy chain variable (IGHV) genes segment IGHV3-7*01 up to the amino acid C105, prior to the CDR-H3 (International Immunogenetics Information System [IMGT])⁵². However, position G106—the site of the junction between IGHV and the IGH diversity (IGHD) genes—differed from the germline reference. Interestingly, this region is part of a segment (N1) with non-germline nucleotides corresponding to six amino acids identified between the IGHV and IGHD genes (FIG. 16C). This segment is likely the result of N nucleotide additions generated during B cell Ig gene rearrangement, prior to antigen selection. Because of the lack of mutations elsewhere in the sequences, it is likely that the R106G substitution was also generated during this developmental step. The downstream sequence corresponding to the junction between IGHD3-22*01 and the IGH joining (IGHJ) IGHJ6*02 genes also revealed similar nucleotide insertions. Likewise, the Kappa (K) chain junction between the IGKV1-8*01 and IGKJ4*01 genes also contained one insertion. Although we cannot rule out the possibility of SHM-mediated nucleotide changes in the N insertion regions, no mutation was identified in the remainder of the regions of the heavy and light chains. Thus, the P1F12 mAb is likely very close or identical to the original V-(D)-J gene rearrangement in the naïve B cell before antigen contact.

P1F12 Recognizes an Immunodominant Epitope on ZIKV

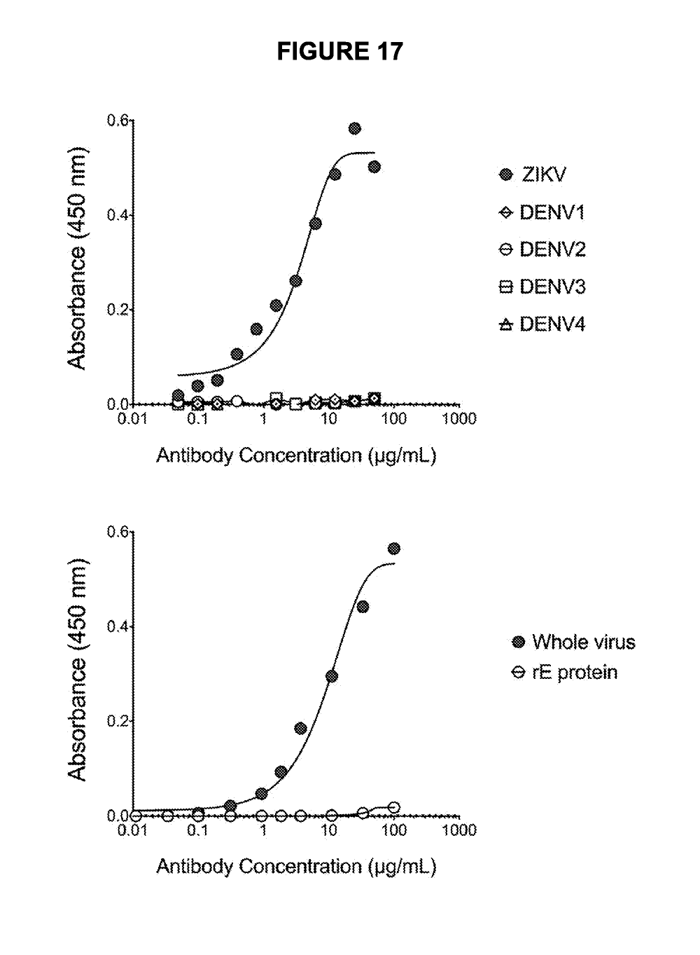

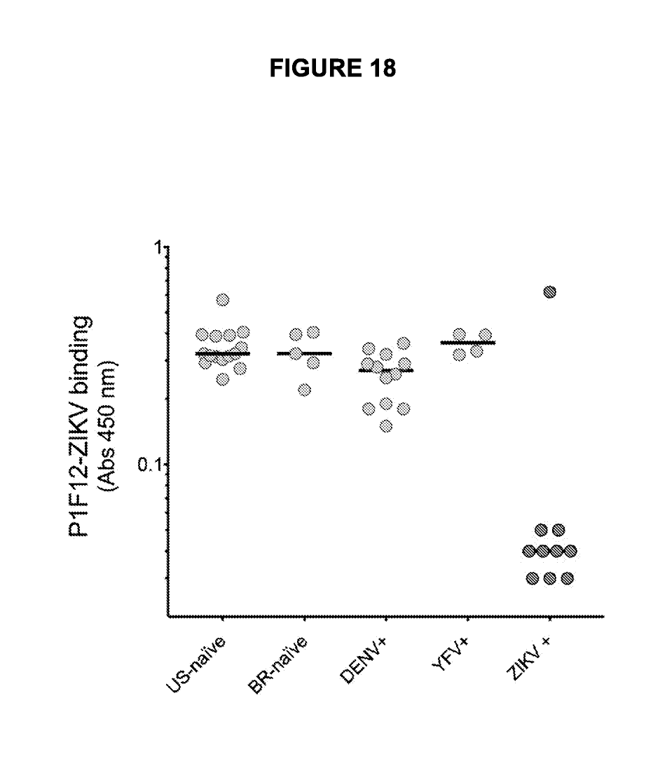

To investigate whether P1F12 recognizes an immunodominant ZIKV epitope, we used a serological blocking assay. In brief, this assay detects the presence of competing Abs that can inhibit the P1F12 mAb binding to its epitope. Because P1F12 did not bind to recombinant E protein (FIG. 17) we used whole virus in our binding assays. We captured ZIKV on the plate using the 4G2 mAb (pan-Flavivirus), and incubated ZIKV with plasma from patients with diverse histories of DENV and ZIKV exposure (TABLE 4). We added unlabeled P1F12 (engineered with rhesus IgG1 constant regions) and detected binding of the mAb using a HRP-labeled mouse anti-rhesus mAb (FIG. 18). Nine of ten plasma samples from individuals that had been infected with ZIKV blocked the binding of P1F12 in a blinded test (FIG. 18, TABLE 4). Similar blocking activity was observed regardless of whether individuals had been previously infected with DENV or had been vaccinated for yellow fever. In contrast, little or no blocking activity was observed by DENV+ plasma in the absence of prior ZIKV exposure (FIG. 18). Furthermore, this recognition was specific in that it was not observed in 14 of 14 DENV-only infected individuals. Thus, the P1F12 serological blocking assay accurately predicted previous ZIKV exposure, as confirmed by RT-PCR, in all but one of the patient plasma samples tested. Although this patient, donor 1302, had a positive urine RT-PCR result for ZIKV, plasma from 1302 did not block P1F12 binding to ZIKV (TABLE 4). Interestingly, the plasma did not exhibit detectable ZIKV-neutralizing activity, suggesting that this patient did not mount a measurable antibody response against ZIKV. In conclusion, only the plasma that inhibited ZIKV infection of Vero cells contained P1F12-blocking antibodies.

TABLE 4

| | | | | | | | P1F12-ZIKV |
|---|---|---|---|---|---|---|---|
| | | | | Previous | Previous YF | Days post most | inhibition assay |
| Sample^{a} | Donors ID | Sex | Age | dengue^{b} | immunization^{b} | recent exposure^{c} | (Abs₄₅₀) |
| ZIKV+ | hu004^{d} | F | 26 | No | No | 46 | 0.04 |
| | 1302^{e} | M | 35 | No | Yes | 110 | 0.62 |
| | 801 | F | 53 | Yes | Yes | 19 | 0.04 |
| | 802 | F | 29 | No | Yes | 19 | 0.05 |
| | 533 | F | 56 | Yes | Yes | 159 | 0.03 |
| | 3002 | F | 51 | No | Unk | 19 | 0.04 |
| | 3004 | F | 59 | Yes | Unk | 19 | 0.03 |
| | 3012 | M | 60 | Yes | Unk | 17 | 0.05 |
| | 3013 | F | 72 | Yes | Unk | 16 | 0.03 |
| | 3018 | F | 43 | No | Unk | 15 | 0.04 |
| DENV+^{f} | 138 | F | 56 | Unk | No | 33 | 0.34 |
| | 152 | M | 87 | No | Yes | 30 | 0.15 |
| | 168 | F | 32 | No | No | 32 | 0.32 |
| | 177 | M | 21 | No | No | 29 | 0.18 |
| | 184 | M | 23 | No | No | 29 | 0.26 |
| | 507 | F | 47 | No | No | 33 | 0.28 |
| | 1235 | M | 17 | No | No | 30 | 0.18 |
| | 1208 | F | 33 | No | Unk | 30 | 0.25 |
| | 1248 | M | 30 | No | Unk | 31 | 0.36 |
| | 1252 | F | 43 | No | Yes | 31 | 0.19 |
| | 1280 | F | 38 | No | No | 34 | 0.29 |
| | 1294 | F | 28 | No | No | 30 | 0.29 |
| YF | FA2002 | M | 34 | Unk | Yes | 28 | 0.319 |
| vaccine | FA2004 | F | 43 | No | Yes | 28 | 0.393 |
| | FA2005 | M | 30 | Unk | Yes | 28 | 0.3315 |
| | FA7006 | M | 73 | Unk | Yes | 28 | 0.396 |
| Brazilian | 12 | F | 27 | Yes | Yes | N/A | 0.22 |
| naïve | 515 | F | 18 | No | Yes | N/A | 0.293 |
| | 1255 | F | 18 | No | Yes | N/A | 0.323 |
| | 1261 | F | 15 | Unk | Unk | N/A | 0.4045 |
| | 1273 | M | 62 | No | Yes | N/A | 0.395 |
| U.S. naïve | hu002 | M | 28 | No | No | N/A | 0.57 |
| | Class 1 | F | 20 | No | No | N/A | 0.3225 |
| | Class 2 | F | 19 | No | No | N/A | 0.3125 |
| | Class 3 | F | 23 | No | No | N/A | 0.4055 |
| | Class 4 | F | 18 | No | No | N/A | 0.392 |
| | Class 5 | F | 19 | No | No | N/A | 0.3885 |
| | Class 6 | F | 21 | No | No | N/A | 0.2925 |
| | Class 7 | F | 22 | No | No | N/A | 0.246 |
| | Class 8 | M | 20 | No | No | N/A | 0.3035 |
| | Class 9 | M | 19 | No | No | N/A | 0.316 |
| | Class 10 | M | 20 | No | No | N/A | 0.3145 |
| | Class 11 | M | 21 | No | No | N/A | 0.3945 |
| | Class 12 | F | 27 | No | No | N/A | 0.344 |
| | Class 13 | F | 24 | No | No | N/A | 0.2755 |
| | Class 14 | M | 24 | No | No | N/A | 0.3225 |

^{a}ZIKV and DENV samples determined by RT-PC;
^{b}Donor reported;
^{c}Days after the onset of symptoms or YF immunization;
Unk, unknown; N/A, Not applicable.
^{d}Patient was not ZIKV RT-PCR positive, but had traveled to a ZIKV endemic area during the outbreak, had symptoms that matched ZIKV infection, and was found to have a plasma Neut₅₀ titer of 1:7,211 against ZIKV at time point collected.
^{e}Patient urine was positive by RT-PCR, but patient had no ZIKV-neutralizing serum activity in the sample tested.
^{f}All donors were positive for DENV1.

Discussion

Here we show that a IgG mAb with no detectable SHM was generated against ZIKV early in infection. Remarkably, despite being germline-encoded, this mAb is ZIKV-specific and does not bind to any of the four DENV serotypes. Furthermore, this mAb not only neutralizes ZIKV, but it also binds to an immunodominant epitope on the virus. Remarkably, despite being germline-encoded, P1F12 binds specifically to ZIKV and does not cross-react with any of the four DENV serotypes. Our results also suggest that P1F12 recognizes a unique epitope on ZIKV. It is unclear how this Ab developed such specificity without SHM. Finally, these findings suggest that affinity maturation is not necessary for the generation of isotype switched virus-neutralizing Abs.

Low levels of SHM in Abs possessing neutralizing activity have been previously reported in mice and humans⁵³⁻⁵⁵, supporting the idea that germline-encoded mAbs can indeed neutralize. Abs with low levels of SHM have also been reported during the acute phase of human DENV infection, but it was not clear that these Abs contributed to the antiviral neutralization activity⁵⁶. In studies in mice, VSV-specific mAbs lacking SHM have been isolated previously⁵³. Interestingly, secondary, but not primary, mouse Abs against VSV had mutations⁵⁷. Furthermore, the reversion of these mutated Abs to non-mutated precursors reduced, but did not abrogate, VSV binding and neutralizing activity. The binding differences between the mutated and germline Abs were much less pronounced than might be expected⁵⁷. Additionally, mice that cannot conduct SHM due to AID knockout still mounted neutralizing Ab responses against Friend virus, a strain of murine leukemia virus⁵⁵. It has been suggested that these Abs lacking extensive SHM undergo a GC-independent developmental pathway⁵⁸, although the mechanistic basis for this phenomenon remains to be elucidated.

Rapid, GC-independent responses might be particularly relevant in the control of acute cytopathic viruses^{55, 58, 59}. The GC-independent Abs would arise quickly after infection and then curtail viral replication, preventing virus-mediated damage⁶⁰. Even more provocatively, Hangartner et al. have argued that cytopathic viruses specifically evolved to retain binding to these germline sequences to decrease host lethality and increase fitness. On the other hand, chronic viruses may have evolved to avoid germline-binding and development of neutralizing responses to persist⁶⁰. So far, these hypotheses remain unsubstantiated by the lack of evidence for strictly germline neutralizing Ab responses in humans. While our experiments were not specifically designed to detect GC-independent responses, it seems likely that the isotype-switched P1F12 originated directly from a germline precursor.

We isolated P1F12 from a ZIKV-infected individual that developed neurological complications compatible with GBS and was treated with IVIG. Underlying factors that influence the potential association of GBS and ZIKV infection might involve an autoimmune process, which could influence the development of immune responses⁶¹. Additionally, IVIG may have had a role in the selection of the Ab responses mounted by peripheral B cell repertoires⁶². This is unlikely, however, since the patient initiated IVIG treatment on the same day that the plasmablasts were isolated. It is possible, then, that GBS or IVIG-treatment influenced the development of P1F12. These potential associations are difficult to determine and were outside the scope of this study. It is clear, however, that these responses were not exclusive to volunteer 533, as P1F12 binding can be blocked by the serum of most ZIKV-infected individuals (FIG. 18).

Recently described ZIKV-specific mAbs derived from Epstein-Barr virus-immortalized memory B cells are highly polyclonal and have undergone SHM⁴². However, SHM levels in these human anti-ZIKV mAbs were lower than SHM levels in mAbs isolated in response to primary infections or vaccination (SARS-CoV, H5N1, rabies vaccine), recurrent or chronic infections (RSV, PIV, Staphylococcus aureus, Klebsiella pneumoniae, HCMV, HCV) or autoimmune diseases⁴². Wang et al. have recently reported the isolation of 13 new ZIKV-specific mAbs from memory B cells, three of which had very little SHM⁴⁵. These mAbs were isolated from memory cells sorted with soluble and monomeric ZIKV E proteins and, in contrast to P1F12, bind to the recombinant protein⁴⁵. In contrast, we isolated ZIKV− specific mAbs from circulating plasmablasts at D12. The peak recall of memory B-cell derived plasmablasts is thought to occur within the first week post-secondary infection^{63,64}. Thus, it is probable that most of the isolated mAbs did not have a memory-B cell origin, and it remains possible that some of the plasmablasts were sorted from the basal population that circulate in low frequencies in the blood. In conclusion, the isolation of mAbs using different B cell methods suggest that anti-ZIKV mAbs with germline characteristics are not limited to specific B cell subtypes^{42,45}. Notably, the anti-ZIKV mAbs isolated to date are less mutated than the mAbs isolated after related DENV infections⁴²⁻⁴⁵. Together, these findings suggest possible differences in the development of Ab responses against ZIKV.

Unfortunately, despite our efforts, we were unable to map P1F12's binding site. We first employed an in vitro escape assay⁶⁵, which did not result in a single mutated consensus sequence. Also, P1F12 did not bind to the prM/E proteins expressed in cells, precluding our ability to map this interaction using an Ala-mutated envelope panel^{66,67}. Characterizing this interaction will, thus, require a significant effort that is beyond the scope of the current manuscript. Because the P1F12 mAb retains the ability to bind virions, our conclusion is that it binds to a conformational epitope.

Based on the cohort of human plasma samples tested in this study, it appears that most ZIKV− infected individuals mount Ab responses against the epitope recognized by P1F12. This epitope is recognized by Abs in individuals previously infected by ZIKV, thereby preventing the binding of P1F12. By contrast, Abs in the plasma from individuals previously infected by any of the DENV serotypes, do not prevent binding of P1F12. P1F12 may, therefore have potential as a diagnostic. Several diagnostic options for testing for ZIKV exposure exist, including RT-PCR, IgM ELISA, and PRNT methods^{22,68}. While it is relatively straightforward to detect ZIKV nucleic acid during the acute phase in blood, urine, saliva, and semen, it has proven more difficult to design rapid and effective diagnostics for ZIKV exposure in the chronic phase. For samples collected after the first week of symptoms, the initial test is an anti-ZIKV, anti-DENV, anti-CHIKV virus IgM ELISA⁶⁸. However, in patients who have received a flaviviral vaccine (DENV, YFV, or JEV) and/or have been infected with any Flaviviruses in the past, these assays may be difficult to interpret due to the cross-reactivity of the Abs⁶⁸⁻⁷³. Thus, a positive IgM test needs to be confirmed with a laborious PRNT assay. IgM antibodies persist for 2-12 weeks in serum, and sera from individuals previously infected for more than 12 weeks would also have to be confirmed with a virus neutralization-based method⁶⁸. Our plasma inhibition assay may, perhaps, provide an alternative to these other techniques.

In this study, we isolated plasmablast-derived Abs from a ZIKV-infected individual with unusual characteristics. The human IgG P1F12 has no or limited SHM yet binds to an immunodominant ZIKV epitope that is not present on any of the four DENV serotypes. Furthermore, this mAb can neutralize the virus with a Neut₅₀ of approximately 2 μg/ml. Our results suggest that SHM-independent pathways may generate neutralizing Abs in the responses against ZIKV.

### Example 9

The following example describes the methods and materials used in the study of Example 7.

Human Samples

Blood samples were collected from volunteer 533, a 56-year-old woman who reported a pruriginous skin rash that started six days prior to the beginning of acute neurological deficits suggestive of GBS. ZIKV infection was confirmed by a positive real-time reverse-transcriptase PCR assay for ZIKV RNA in urine samples collected at days 11 and 12 after the onset of the first rash symptoms. Blood and cerebrospinal fluid were negative for ZIKV RNA. Previous history of a single dengue infection and yellow fever immunization were also reported. Peripheral blood mononuclear cells (PBMCs) were obtained from blood samples collected 12 days post onset of symptoms. Blood samples from patient 533 were obtained after signing a written consent form approved by the University of São Paulo's Institutional Review Board (CAPPesq 0652/09). Anonymized plasma samples from volunteers in Brazil and US were obtained from naïve and convalescent subjects with RT-PCR-confirmed ZIKV or DENV infection (TABLE 4). Four volunteers donated samples post yellow fever vaccination.

Flow Cytometry and Plasmablast Sorting

We determined the frequency of plasmablasts in circulation by cytometric analysis of PBMCs obtained from blood collected in acid citrate dextrose (ACD) using a Ficoll-Paque (GE Lifesciences) gradient. Briefly, we stained fresh PBMC samples (1×10⁶ cells, room temperature, in the dark), with 100 μl of a cocktail containing the following fluorophore-antibody conjugates: phycoerythrin (PE)-CF594 anti-human CD3 (clone UCHT1; Becton Dickinson [BD]), PE-CF594 anti-human CD14 (clone MϕP9; BD), Allophycocyanin (APC)-Cyanine (Cy)7 anti-human CD19 (clone SJ25C1; BD), Peridinin Chlorophyll Protein Complex (PerCP) anti-human CD20 (clone L27; BD), APC anti-human CD27 antibody (clone 0323; Biolegend), Fluorescein isothiocyanate (FITC) anti-human CD38 (clone HB7; BD), PE anti-human CD138 (clone M115, BD). We also included the fixable viability dye LIVE/DEAD® Fixable Red Dead Cell Stain Kit (Life Technologies) in the staining mix, in order to discriminate between live and dead cells. After 30 min, we washed the cells twice with FACS buffer (PBS, 0.5% FBS, 2 mM EDTA), resuspended with a PBS 1× solution, and stored at 4° C. until acquisition on the same day. Samples were acquired using a BD FACSAria IIu flow cytometer and analyzed using FlowJo 9 (FlowJo). The plasmablast population was defined as live CD19+ CD3− CD14− CD20− CD27+ CD38+ cells (see gating and sort strategy in Figure of Magnani et al., PLoS Negl Trop Dis 11(6): e0005655 (2017)). Using this same plasmablast staining, fresh PBMC samples (5×10⁶ cells) were sorted on a BD FACSAria II flow cytometer. Single plasmablast cells were sorted into 96-well plates containing a lysis buffer designed to extract and preserve the RNA (250 mM Tris-HCl pH 8.3, 375 mM KCl, 15 mM MgCl₂, 6.25 mM DTT, 250 ng/well yeast tRNA, Life Technologies; 20 U RNAse inhibitor, New England Biolabs [NEB]; 0.0625 μl/well IGEPAL CA-630, Sigma). After sorting, the RNA plates were immediately frozen in dry ice for subsequent cloning of the Ab chains.

Ab Repertoire Analysis

We conducted reverse transcription followed by a nested PCR to amplify the variable region of the Immunoglobulin (Ig) chains using described protocols with minor modifications⁴⁶. Briefly, cDNA was synthesized in a 25 μl reaction using the original sort plates. Each reaction contained 1 μl of 150 ng random hexamer (IDT), 2 μl of 10 mM dNTP (Life Technologies), 1 μl of SuperScript III Reverse Transcriptase (Life technologies), 1 μl molecular biology grade water, and 20 μl of single sorted cell sample in lysis buffer (described above). The reverse transcription reaction was performed at 42° C. for 10 min, 25° C. for 10 min, 50° C. for 60 min, 94° C. for 10 min. After the reaction was completed, cDNA was stored at −20° C. Heavy and light chains were amplified in three different nested PCR reactions, using a mix of 5′ V-specific primers with matching 3′ primers to the constant regions of IgG, IgL, and IgK. PCR reactions were conducted using HotStarTaq Plus DNA Polymerase (Qiagen). The second set of PCR reactions was carried out with primers redesigned to incorporate restriction sites compatible with subcloning into rhesus IgG1 expression vectors, instead of the original human vectors⁴⁶. We sequenced the amplified and cloned products using primers complementary to the Ig constant regions. Sequences were analyzed using IgBLAST and IMGT/V-QUEST to identify V (D) J gene rearrangements, as well as SHM levels^{47,48}.

Ab Expression and Purification

We expressed mAbs in Expi293F (ThermoFisher) human cell lines. The plasmids encoding heavy and light chains were co-transfected using the ExpiFectamine 293 Transfection kit (A14525, ThermoFisher). After 5-6 days, we harvested the secreted mAb in the supernatant. Ig concentration in the supernatant was determined by an anti-rhesus IgG ELISA, before we proceeded with the functional assays. For the experiments with purified mAbs, we used Protein A Plus (Pierce)-containing columns to remove the impurities. The concentration of purified protein was determined by measurement of absorbance at 280 nm (NanoDrop, Thermo Scientific).

Virus Capture Assay and Recombinant E Protein ELISA

P1F12 binding was determined by both virus capture assay (VCA) and recombinant (r)E ELISAs. The VCA plates were coated overnight with the mouse-anti-Flavivirus monoclonal antibody 4G2 (clone D1-4G2-4-15, EMD Millipore) followed by incubation with viral stocks (ZIKV or DENV). The rE ELISA plates were coated with ZIKV E Protein (MyBiosource, MBS596001) diluted to 5 μg/ml in PBS. After the coating step, the plates were washed with PBS and mAb samples diluted to 1 μg/ml were added to designated wells and incubated for 1 h at 37° C. Subsequently, the plates were washed and detection was carried out using a goat anti-human IgG HRP secondary Ab (Southern Biotech), which was added to all wells at a dilution of 1:10,000. Following a 1 h incubation at 37C, the plates were washed and developed with TMB substrate at room temperature for 3-4 min. The plates were developed with TMB substrate at room temperature for 3-4 min. The reaction was stopped with TMB solution and absorbance was read at 450 nm.

Flow Cytometry-Based Neutralization Assay

The neutralizing potency of the mAbs was measured using a flow cytometry-based assay^{49,50}. In brief, recombinant mAbs (transfection supernatant or purified) were diluted and pre-incubated with ZIKV (Paraiba) or the reference DENV serotypes in a final volume of 220 μL for 1 h at 37° C. The virus and mAb mixture (100 μL) was added onto wells of a 24-well plate of 100% confluent Vero cell monolayers in duplicate. A new seed of Vero cells (CCL-81TM) was obtained from the American Type Culture Collection (ATCC) repository for this study. The inoculum was incubated in a 37° C. incubator at 5% CO₂ for one hour with agitation of the plates every 15 min. After one hour, the virus and mAb-containing supernatants were aspirated and the wells were washed with media. Fresh media was then added and the plates were incubated for a total of 24 hours. Cells were trypsinized with 0.5% trypsin (Life Technologies), fixed (BD cytofix), and permeabilized (BD cytoperm). Viral infection was detected with the 4G2 antibody (Millipore) recognizing ZIKV or DENV, followed by staining with an anti-mouse IgG2a APC fluorophore-conjugated secondary reagent (Biolegend). The concentration to achieve half-maximal neutralization (Neut₅₀) was calculated using a nonlinear regression analysis with Prism 7.0 software (GraphPad Software, Inc.). The following strains were used in our neutralization assays: ZIKV Paraiba 2015 (KX280026.1), DENV1-West Pac (U88535.1), DENV2-NGC (AF038403.1), DENV3-Sleman/78 (AY648961), and DENV4-Dominica (AF326573.1)

Plaque Reduction Neutralization Test (PRNT)

PRNTs were conducted as previously described⁵¹. Briefly, purified P1F12 was serially diluted in OptiMEM supplemented with 2% human serum albumin (VWR), 2% fetal bovine serum, and gentamicin. ZIKV Paraiba 2015 was diluted to a final concentration of ^{˜}500-1000 PFU/mL in the same diluent added to equal volumes of the diluted Ab. The virus/mAb mixture was incubated at 37° C. for 30 min. Cell culture medium was removed from 90% confluent monolayer cultures of Vero cells on 24-well plates and 100 μl of the virus/Ab mixture was transferred onto duplicate cell monolayers. Cell monolayers were incubated for 60 min at 37° C. and overlaid with 1% methylcellulose in OptiMEM supplemented with 2% FBS 2 mM glutamine+50 μg/ml gentamicin. Samples were incubated at 37° C. for four days after which plaques were visualized by immunoperoxidase staining, and a 50% plaque-reduction neutralization titer was calculated.

P1F12-ZIKV Binding Inhibition Assay

Inhibition of P1F12 mAb binding was determined by ELISA. To begin, the ELISA plate was coated with mouse anti-Flavivirus monoclonal antibody 4G2 (EMD Millipore) diluted 1:1,000 in carbonate binding buffer and incubated overnight at 4° C. The next day, the plate was washed five times with PBS-Tween20 and wells were blocked with 5% skim milk in PBS for 1 h at 37° C. After the block step, the plate was washed and virus samples were added to designated wells for 1 h incubation at room temperature. Subsequently, the plate was washed with PBS only and corresponding blocking plasma samples were added for 1 h at 37° C. Following the plasma block, the plate was washed and P1F12 was added to corresponding wells for 1 h at 37° C. P1F12 was detected using a rhesus IgG-specific antibody (mouse anti-monkey IgG-HRP clone SB108a; Southern Biotech). Thereafter, the plate was washed and wells were developed with TMB substrate at room temperature for 3-5 min before the reaction was stopped with TMB Stop Solution. Absorbance was determined at 450 nm.

### Example 10

Zika virus (ZIKV) has caused the world to take note of long forgotten tropical diseases¹⁻². Diagnostic tests are critical for the accurate diagnosis of ZIKV, and the ability to accurately distinguish ZIKV from other tropical diseases has been challenging³. ZIKV is closely related to several other tropical arboviruses including West Nile virus (WNV), Yellow Fever virus (YFV), and most importantly dengue virus (DENV)⁴. Many people infected with these other arboviruses can often be misinterpreted as having ZIKV and vice versa. The proper diagnosis of ZIKV is of paramount importance, as ZIKV is the only virus among these other diseases that can have higher rates of adverse fetal outcomes⁵. Furthermore, with only 20% of infected individuals showing any symptoms, knowledge of ZIKV sero-status is critical for family planning and epidemiological purposes⁶.

While the 15 FDA EUA approved PCR based tests have shown excellent results in diagnosing ZIKV when the virus is present, they often miss the short window in which the virus is present in the blood and urine^{7,8}. This brief window of opportunity for acute diagnosis means that the bulk of diagnostic testing will need to be performed serologically. Because ZIKV and DENV are both from the flavivirus family, they share many structural features, making serological diagnosis particularly challenging⁴. False-positives are the norm, with the highest cross-reactivity seen in secondary DENV infections⁹.

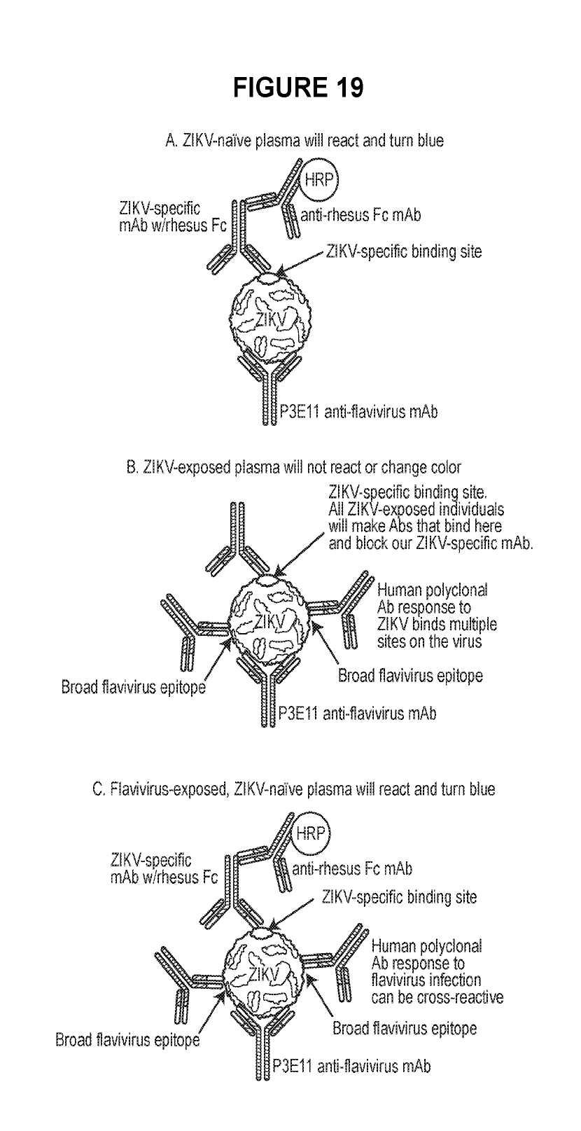

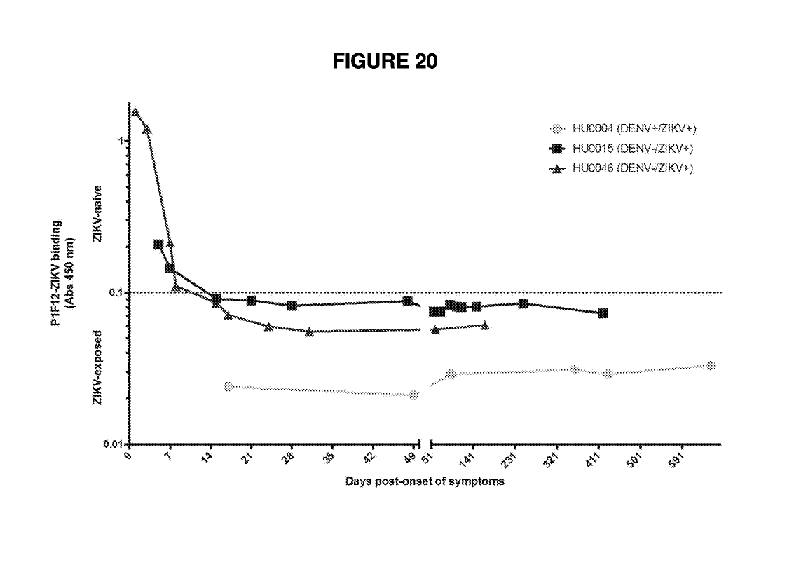

Fortunately, we have developed a serological ELISA test (the P1F12 test) to determine prior ZIKV infection with the previously isolated ZIKV-specific monoclonal antibody (mAb) P1F12¹⁰. The P1F12 test is based on the principle of an immunodominant Ab response to ZIKV, and the patient's plasma blocking the binding of our highly ZIKV-specific P1F12 mAb to whole ZIKV (FIG. 19). If the patient is P1F12 test positive, the patient has not developed a ZIKV-specific immune response. Even in cases of other prior flavivirus exposure, this unique P1F12 binding site remains open and available for our P1F12 mAb to bind. If the patient is P1F12 negative, then the patient has been previously exposed, as they have mounted a ZIKV-specific immune response and blocked the unique P1F12 binding site. We also tested several patients during the acute and convalescent phase with serial draws to determine when our test becomes effective (FIG. 20). Here, we see that among patients tested, after day 14 post-onset of symptoms, the blocking response is established and does not decrease with time.

More than 809 blinded convalescent (day 10 post-onset of symptoms or later) clinical samples from the United States as well as from different parts of Brazil (Table 5) were tested. Of these clinical samples, 112 were true ZIKV positives, as determined by either clinical ZIKV-PCR or ZIKV-FRNT testing. The test was accurate in detecting prior ZIKV infection, as there were only 4 false positives and 5 false negatives from the 809 samples tested. We recorded values of 0.96 and 0.99 for sensitivity and specificity respectively (Table 6). The positive predictive value was 0.97 and the negative predicative value was 0.99.

TABLE 5
Human plasma or sera samples tested for ZIKV.

| | | | | ZIKV | ZIKV | False | False |
|---|---|---|---|---|---|---|---|
| | Country of | | History | posi- | nega- | posi- | nega- |
| Group | Origin | Samples | of DENV | tive* | tive | tive | tive |
| Fleury | Brazil | 38 | 19 | 8 | 27 | 3 | 0 |
| NIH | USA | 51 | 15 | 9 | 42 | 0 | 0 |
| Esper21 | Brazil | 21 | 12 | 7 | 13 | 1 | 0 |
| Brazil37 | Brazil | 37 | 16 | 10 | 25 | 0 | 2 |
| Santos | Brazil | 175 | 165 | 71 | 101 | 0 | 3 |
| FDA | USA | 175 | 15 | 0 | 175 | 0 | 0 |
| Miami | USA | 312 | 3 | 7 | 305 | 0 | 0 |
| Totals | X | 809 | 245 | 112 | 688 | 4 | 5 |

*Samples were determined ZIKV positive through clinical ZIKV-PCR or ZIKV-PRNT tests.

TABLE 6
Statistical analysis of tests performed

| Statistic | Result |
|---|---|
| ZIKV Sensitivity | 0.957 |
| ZIKV Specificity | 0.993 |
| Positive | 0.965 |
| predictive value | |
| Negative | 0.991 |
| predictive value | |

Since ZIKV co-circulates with other diseases, we tested a large panel of other common infections (3-15 different samples of each) with symptoms similar to ZIKV (Table 7). These include WNV, DENV, Chikungunya, Malaria (Plasmodium falciparum), Parvovirus B-19, Varicella zoster virus, Epstein-Barr virus, Cytomegalovirus, Hepatitis C, anti-Rheumatoid factor, St. Louis encephalitis virus, anti-nuclear antibody, YFV vaccines, and Japanese Encephalitis virus exposed plasma or sera samples. All samples were tested for Ab against HIV 1/2, HBsAg, HIVag, HIV 1 RNA, HBV DNA, ZIKV RNA and found to be negative. Each sample was from a different donor and had been characterized positive for the given disease state using a FDA-approved test.

TABLE 7
Potentially confounding disease state samples

| | | No. | ZIKV+ |
|---|---|---|---|
| | | of | with |
| | | Sam- | P1F12 |
| Disease State Sample | Clinical Details | ples | test |
| Cytomegalovirus | IgM+/IgG+ plasma | 4 | 0/4 |
| Epstein-Barr virus | IgM+/IgG+ plasma | 4 | 0/4 |
| Parvovirus B19 | IgM+/IgG+ plasma | 3 | 0/3 |
| Varicella zoster virus | IgM+/IgG+ plasma | 3 | 0/3 |
| Malaria | IgM+/IgG+ plasma | 3 | 0/3 |
| (plasmodium falciparum) | | | |
| Chikungunya virus | IgM+/IgG+ plasma | 8 | 0/8 |
| Dengue virus | IgM+/IgG+ plasma | 15 | 0/15 |
| Yellow Fever virus | IgM+/IgG+ plasma | 15 | 0/15 |
| (vaccine recipients) | >30 days | | |
| | post-immunization | | |
| West Nile virus | IgM+/IgG+ plasma | 15 | 0/15 |
| Rheumatoid factor | 1001-2000 IU/mL | 3 | 0/3 |
| | plasma | | |
| Japanese Encephalitis virus | IgM+/IgG+ plasma | 1 | 0/1 |
| St. Louis Encephalitis virus | IgM+/IgG+ plasma | 3 | 0/3 |
| Anti-nuclear antibody (ANA) | IgM+/IgG+ plasma | 3 | 0/3 |

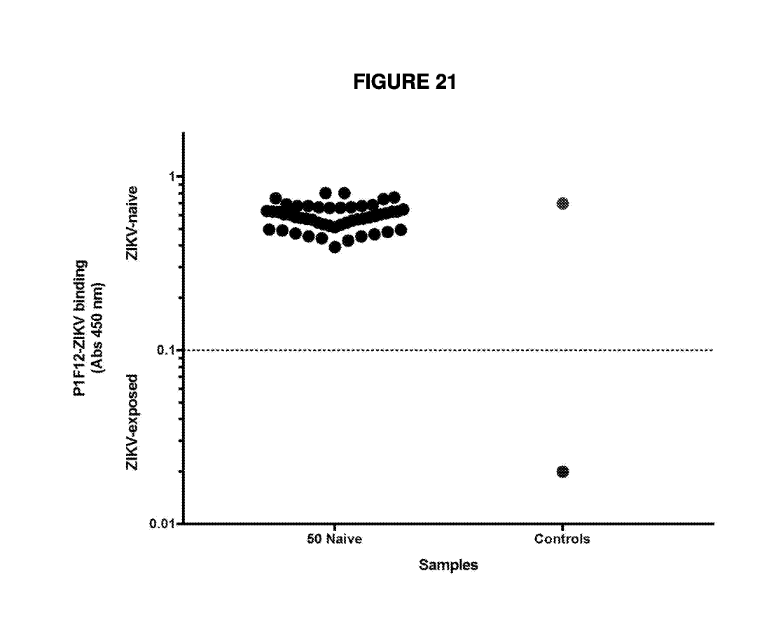

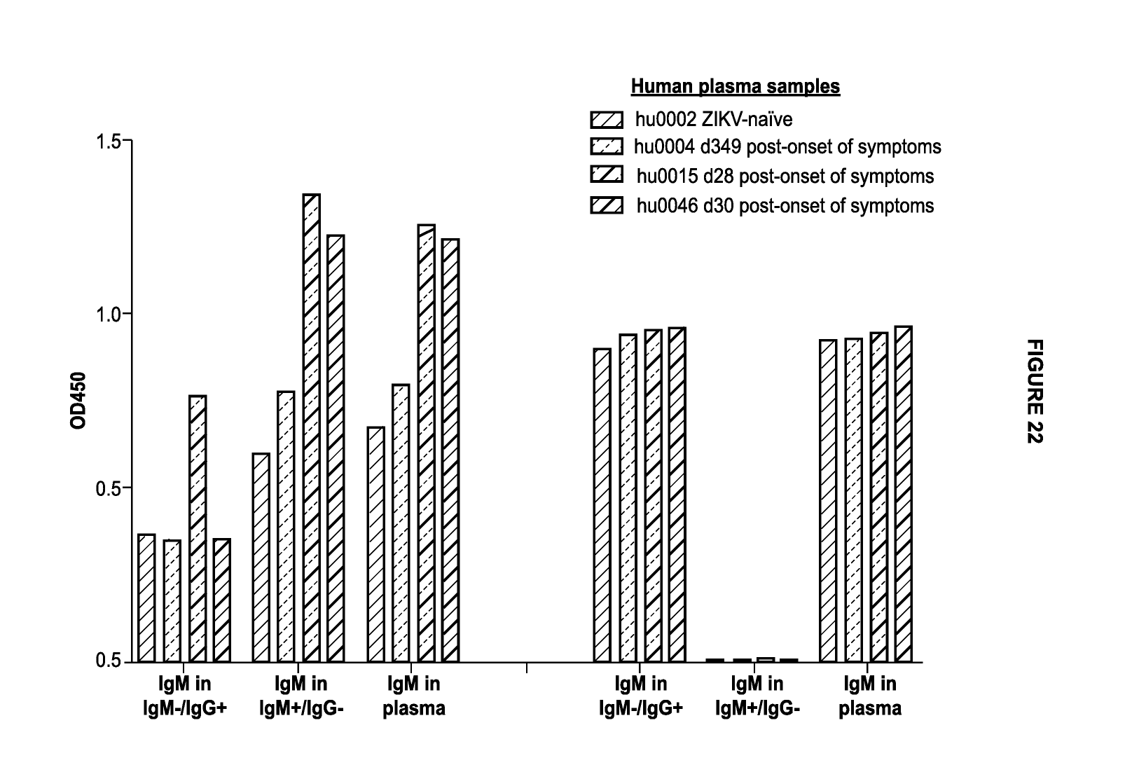

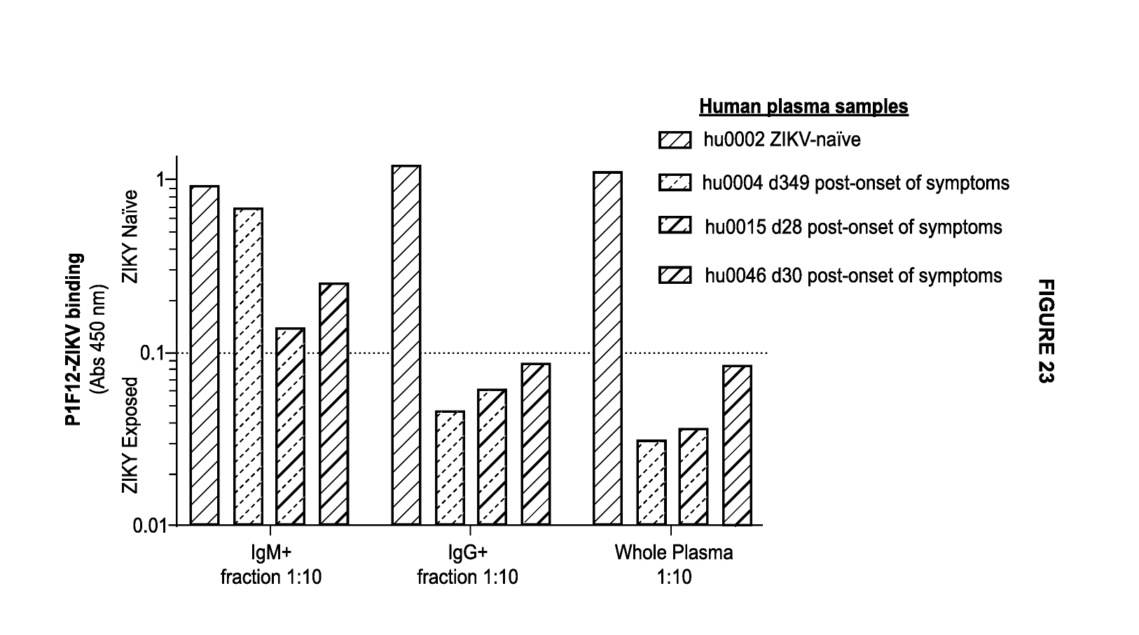

In addition to testing other co-circulating diseases, we also tested various concentrations of commonly found potentially interfering substances which were spiked into 4 separate human ZIKV naïve plasma samples (Table 8). These substances included hemoglobin, bilirubin, cholesterol, rheumatoid factor, humanized anti-mouse antibodies (HAMA), and albumin. Lastly, we tested 50 ZIKV true naïve individuals from an area without local ZIKV infection (FIG. 21). All samples were found to be negative for our diagnostic P1F12 test, thus limiting the risk of false positives.

TABLE 8
Potentially interfering substance samples

| Interfering | Concentrations | ZIKV+ |
|---|---|---|
| Substances | tested | with P1F12 test |
| Hemoglobin | 250 mg/mL; 125 mg/mL; 75 mg/mL | 0/3; 0/3; 0/3 |
| Albumin | 250 mg/mL; 125 mg/mL; 75 mg/mL | 0/3; 0/3; 0/3 |
| Cholesterol | 10 mg/mL; 5 mg/mL; 2.5 m/mL | 0/3; 0/3; 0/3 |
| Bilirubin | 1 mg/mL; 0.5 mg/mL; 0.25 mg/mL | 0/3; 0/3; 0/3 |
| Human anti- | 1 mg/mL; 0.5 mg/mL; 0.25 mg/mL | 0/3; 0/3; 0/3 |
| mouse antibody | | |
| (HAMA) | | |

The entire P1F12 test can be performed with as little as 25 μL of plasma or serum in several hours, and with automation, hundreds of tests can be performed in a single day with remarkable reproducibility. In stark contrast to the four currently FDA EUA approved ZIKV-IgM tests, our test is strikingly specific. The current ZIKV-IgM tests have high cross-reactivity with the sera from patients with prior and active DENV infections, as well as other co-circulating arbovirus infections since they utilize ZIKV NS1 protein instead of our whole virus particle approach. Furthermore, ZIKV-IgM testing is only viable for the extent of ZIKV-specific IgM in the blood, where our test has shown blocking of IgG for over 1.5 years post infection. Our rapid, highly specific ZIKV diagnostic test has immediate application for the accurate clinical diagnosis of prior ZIKV infection in the millions of at-risk individuals around the world.

### Example 11

This example demonstrates a method of differentiating between new and old ZIKV infection using fractionated plasma.

In addition to the P1F12 test serving as a serological measure of prior ZIKV infection, we have generated preliminary data to support the notion that the P1F12 test may also be used to differentiate new (within 1-6 months post infection) and old (greater than 6 months post infection) ZIKV infection in healthy adults. This is due to the ability of P1F12 to block not only total immunoglobulin (Ig) in plasma, but also the ability to block IgM during the peri-acute phase of ZIKV infection when ZIKV-specific IgM is present in high levels in the patient's plasma.

IgM against ZIKV is expressed roughly 2-5 days post-onset of symptoms and generally remains in circulation for 60-90 days post-onset of symptoms. This is also known as the peri-acute phase. IgG against ZIKV is expressed roughly between days 10-20 post-onset of symptoms and can remains for years. Under the premise of these immunological principles, we attempted to block the IgG fraction of the patients' plasma with our P1F12 mAb. Separately, we also attempted to block the IgM fraction of the patients' plasma with our P1F12 mAb. These test results would allow us to determine if the infection was recent or old.

We took total plasma from a ZIKV-naïve individual (hu0002), a ZIKV-exposed individual (hu0004) approximately one year post infection, and two ZIKV-exposed individuals (hu0015 and hu0046) approximately one month post infection. Using protein G agarose beads, we separated the IgM from the IgG fractions of the plasma. We then ran these separated fractions, and some of the original plasma, in a semi-quantitative Ig ELISA (FIG. 1). Here, we detected IgM in the IgM−/IgG+ fraction, IgM+/IgG− fraction, and total plasma. IgM was retained in the IgM+/IgG− fraction when compared to the total plasma, however, some IgM was also extracted into the IgM−/IgG+ fraction where it should not be. We also saw that in the IgM+/IgG− fraction from all patients, IgG was almost completely removed and in the IgM−/IgG+ fraction it was almost entirely retained. Thus, leaving us with a very pure IgM fraction and a relatively pure IgG fraction with some IgM impurity.

The next step was to run these fractions and total plasma from each patient in our P1F12 test (FIG. 2). Here we saw our ZIKV-naïve sample, hu0002, fail to block P1F12 binding in all fractions. Our ZIKV− exposed sample, hu0004, blocked P1F12 binding in the IgM−/IgG+ fraction and whole plasma, and failed to block the IgM+/IgG− fraction. This is anticipated as the sample from hu0004 was collected 349 days post-onset of symptoms. IgM directed against ZIKV should not still be present in the patient's plasma. Next, hu0015 and hu0046, both recent ZIKV infections, blocked P1F12 binding in the IgM−/IgG+ fraction and whole plasma. Unfortunately, while these samples did not block the IgM+/IgG− fraction as well as the IgG and whole plasma fractions, they did significantly decrease the ability of P1F12 to bind when compared to the naïve control and the one year previously exposed control. These results show promise that the P1F12 test may have the capability, based on running it on IgM and IgG fractions, to differentiate between recent and old ZIKV infections.

The value of this is immense in countries where there has been prior ZIKV outbreaks, as much of the population will already be seropositive. Unfortunately, without a test to distinguish recent and old infections, patients receiving a positive test would likely want to wait to become pregnant as ZIKV can be sexually transmitted by semen for up to 6 months post-infection. Furthermore, there is no test currently available capable of serologically differentiating new and old ZIKV infections.

### REFERENCES

The following is a list of references cited throughout the application, except for Examples 8-10:
1. Brasil P, et al., N Engl J Med. Mar. 4, 2016. Epub ahead of print. PubMed PMID 26943629
2. Mlakar et al., N Engl J Med. 2016; 374(10):951-8.
3. Dick G W. Trans R Soc Trop Med Hyg. 1952; 46(5):521-34.
4. Dick et al., Trans R Soc Trop Med Hyg. 1952; 46(5):509-20.
5. Faye et al., PLoS Negl Trop Dis. 2014; 8(1):e2636.
6. Bearcroft W G. Trans R Soc Trop Med Hyg. 1956; 50(5):442-8.
7. Macnamara F N. Trans R Soc Trop Med Hyg. 1954; 48(2):139-45.
8. Boorman et al., Trans R Soc Trop Med Hyg. 1956; 50(3):238-42.
9. Haddow et al., Bull World Health Organ. 1964; 31:57-69.
10. Bell et al., Arch Gesamte Virusforsch. 1971; 35(2):183-93.
11. Duffy et al., N Engl J Med. 2009; 360(24):2536-43.
12. Hayes E B. Emerg Infect Dis. 2009; 15(9):1347-50.
13. Lanciotti et al., 2007. Emerg Infect Dis. 2008; 14(8):1232-9.
14. Besnard et al., Evidence of perinatal transmission of Zika virus, French Polynesia, December 2013 and February 2014. Euro Surveill. 2014; 19(13); PubMed PMID 24721538.
15. Cao-Lormeau et al., 2013. Emerg Infect Dis. 2014; 20(6):1085-6.
16. Musso et al., Clin Microbiol Infect. 2014; 20(10):0595-6.
17. Oehler et al., Euro Surveill. 2014; 19(9); PubMed PMID 24626205.
18. Dupont-Rouzeyrol et al., Emerging Infectious Diseases. 2015; 21(2):381-2.
19. Roth A, et al., Euro Surveill. 2014; 19(41). PubMed PMID: 25345518.
20. Campos et al., J Clin Virol. 2016; 77:69-70.
21. Gourinat et al., Emerg Infect Dis. 2015; 21(1):84-6.
22. Musso et al., J Clin Virol. 2015; 68:53-5.
23. Musso et al., Emerg Infect Dis. 2015; 21(2):359-61.
24. Foy et al., Emerg Infect Dis. 2011; 17(5):880-2.
25. Tognarelli et al., Arch Virol. 2016; 161(3):665-8.
26. Zanluca et al., Mem Inst Oswaldo Cruz. 2015; 110(4):569-72.
27. Calvet et al., J Clin Virol. 2016; 74:1-3.
28. Campos et al., Emerg Infect Dis. 2015; 21(10):1885-6.
29. Cardoso et al., Emerg Infect Dis. 2015; 21(12):2274-6.
30. Faccini-Martinez et al., J Infect Public Health. 2016; PubMed PMID 26837723.
31. Villamil-Gomez et al., J Infect Public Health. 2016; PubMed PMID 26754201.
32. Dyer O. BMJ. 2015; 351:h6983.
33. Gatherer et al., J Gen Virol. 2016; 97(2):269-73.
34. Hennessey et al., MMWR Morb Mortal Wkly Rep. 2016; 65(3):55-8.
35. Thomas et al., MMWR Morb Mortal Wkly Rep. 2016; 65(6):154-8.
36. Calvet et al., Lancet Infect Dis. 2016; 16(6):653-60.
37. de Paula Freitas B, et al., JAMA Ophthalmol. 2016. doi: 10.1001/jamaophthalmol.2016.0267. PubMed PMID: 26865554.
38. Martines et al., Lancet. 2016. doi: 10.1016/50140-6736(16)30883-2. PubMed PMID: 27372395.
39. Martines et al., MMWR Morb Mortal Wkly Rep. 2016; 65(6):159-60.
40. Mor, Am J Reprod Immunol. 2016; 75(4):421-2.
41. Oliveira Melo A S, et al., Ultrasound Obstet Gynecol. 2016; 47(1):6-7.
42. Sarno et al., PLoS Negl Trop Dis. 2016; 10(2):e0004517.
43. Schuler-Faccini et al., MMWR Morb Mortal Wkly Rep. 2016; 65(3):59-62.
44. Gulland, B M J. 2016; 352:i657.
45. Heymann et al., Lancet. 2016; 387(10020):719-21.
46. McCarthy M. BMJ. 2016; 352:i1180.
47. McCarthy M. BMJ. 2016; 352:i720.
48. Oduyebo et al., MMWR Morb Mortal Wkly Rep. 2016; 65(5):122-7.
49. Oster et al., MMWR Morb Mortal Wkly Rep. 2016; 65(5):120-1.
50. Petersen et al., MMWR Morb Mortal Wkly Rep. 2016; 65(2):30-3.
51. Staples et al., MMWR Morb Mortal Wkly Rep. 2016; 65(3):63-7.
52. http://www.nbcnews.com/storyline/zika-virus-outbreak/section-miami-neighborhood-local-zika-transmission-cleared-officials-say-n623301. Section of Miami Neighborhood with Local Zika Transmission Cleared: Officials 2016.
53. https://www.labcorp.com/wps/portal/lut/p/c1/04_SB8K8xLLM9MSSzPv8xBz9CPOos_hACzO_QCM_IwMLXyM3AyNjMycDU2dXQwN3M6B8JG55AwMCuv088nNT9Qtyl8oBPK_DWAII/d12/d1/L0IDU0NTQ1FvS1VRb0tVUSEvb0NvUUFBSVFKQUFNWWhSbkdjb3dVaFNvSUEhIS9ZQkpKdzQ1NGtzdXIsMHN0eW9RIS83X1VFNMxSTkzME9HUzlwSVMzTzROMk42NigwL3ZpZXdUZXN0L3Nob3dBbGxSZXN1bHRz/?testid=6040089&criterion=Zika+Virus+Comprehensive+Profile%2C+NAA%2C+Serum+and+Urine. 2016
54. https://www.labcorp.com/wps/portal/lut/p/c1/04_SB8K8xLLM9MSSzPy8xBz9CP0os_hACzO_QCM_IwMLXyM3AyNjMycDU2dXQwN3M6B8JG55AwMCuv088nNT9SP1o8zjQ11Ngg09LY0N_N2DjQw8g439TfyM_MzMLAz0Q_QinYGKlvEqKsiNKDfUOVQEAARgwHAI/dl2/d1/L0I.IwmltbUEhL3dQRUIGUUFOTlFBaERhQUVBWEtHL1UNXlsdyEhLzdfVUU0UzFJOTMwT0dTMjBJUzNPNE4yTjY2ODAvdmlid1Rlc3Q!/?testId=6207015. 2016
55. Fields B N, Knipe D M, Howley P M. Fields virology. 5th ed. Philadelphia: Wolters Kluwer Health/Lippincott Williams & Wilkins; 2007.
56. Barba-Spaeth et al., Nature. 2016. doi: 10.1038/nature18938. PubMed PMID: 27338953.
57. Stettler et al., Science. 2016.
58. Balmer et al., Vet Rec. 2015; 177(11):289.
59. Pereira et al., ACS Appl Mater Interfaces. 2010; 2(9):2602-10.
60. Deo et al., Anal Chem. 2001; 73(8):1903-8.
61. Doleman et al., Anal Chem. 2007; 79(11):4149-53.
62. Qu et al., Anal Biochem. 2007; 371(2):154-61.
63. Rowe et al., Bioconjugate Chem. 2007; 18(6):1772-7.
64. Scott et al., Annu Rev Anal Chem (Palo Alto Calif). 2011; 4:297-319.
65. Scott et al., Bioconjugate Chem. 2011; 22(3):475-81.
66. Shrestha et al., Bioconjugate Chem. 2002; 13(2):269-75.
67. Andreasson et al., A practical guide to immunoassay method validation. Front Neurol. 2015; 6. doi: UNSP 179.10.3389/fneur.2015.00179.
68. Newman et al., Anal Chim Acta. 1992; 262(1):13-7.
69. Sajid M, et al., Journal of Saudi Chemical Society. 2015; 19(6):689-705.

The following is a list of references cited throughout Examples 8-9:
1. Faye et al., PLoS Negl Trop Dis. 2014; 8(1):e2636
2. Boorman J P and Porterfield J S. Trans R Soc Trop Med Hyg. 1956; 50(3):238-42.
3. Haddow et al., Bull World Health Organ. 1964; 31:57-69.;
4. Duffy et al., N Engl J Med. 2009; 360(24):2536-43.
5. Hayes E B. Emerg Infect Dis. 2009; 15(9):1347-50.
6. Lanciotti et al., 2007. Emerg Infect Dis. 2008; 14(8):1232-9.
7. Dyer O., BMJ. 2015; 351:h6983
8. Gatherer D, Kohl A. J Gen Virol. 2016; 97(2):269-73.
9. Hennessey M, Fischer M, Staples J E. Zika Virus Spreads to New Areas-Region of the Americas, May 2015-January 2016. MMWR Morb Mortal Wkly Rep. 2016; 65(3):55-8.
10. Brasil et al., The New England journal of medicine. 2016; 375(24):2321-34.
11. Brasil et al., PLoS neglected tropical diseases. 2016; 10(4):e0004636 doi: 10.1371/journal.pntd.0004636
12. Honein et al., JAMA: the journal of the American Medical Association. 2016.
13. Calvet et al., The Lancet infectious diseases. 2016; 16(6):653-60.
14. de Paula Freitas B et al., JAMA Ophthalmol. 2016. doi: 10.1001/jamaopthalmol.2016.0267.
15. Martines et al., Lancet. 2016. doi: 10.1016/50140-6736(16)30883-2
16. Martines et al., MMWR Morb Mortal Wkly Rep. 2016; 65(6):159-60.
17. Mlakar et al., The New England journal of medicine. 2016; 374(10):951-8.
18. Mor G., Am J Reprod Immunol. 2016; 75(4):421-2.
19. Oliveira Melo A S, et al., Ultrasound Obstet Gynecol. 2016; 47(1):6-7.
20. Sarno et al. PLoS Negl Trop Dis. 2016; 10(2):e0004517
21. Schuler-Faccini et al., MMWR Morb Mortal Wkly Rep. 2016; 65(3):59-62. doi
22. Staples et al., MMWR Morb Mortal Wkly Rep. 2016; 65(3):63-7.
23. Petersen et al., MMWR Morb Mortal Wkly Rep. 2016; 65(2):30-3.
24. Oster et al., MMWR Morb Mortal Wkly Rep. 2016; 65(5):120-1.
25. McCarthy M., BMJ. 2016; 352:i1180
26. McCarthy M. BMJ. 2016; 352:i720
27. Oduyebo et al., MMWR Morb Mortal Wkly Rep. 2016; 65(5):122-7.
28. Likos et al., MMWR Morb Mortal Wkly Rep. 2016; 65(38):1032-8.
29. Qiu et al., Nature. 2014; 514(7520):47-53.
30. Corti et al., Science. 2016; 351(6279):1339-42.
31. Hessell et al., Nature. 2007; 449(7158):101-4.
32. Eisen H N. Cancer Immunol Res. 2014; 2(5):381-92.
33. Tiller et al., Immunity. 2007; 26(2):205-13.
34. West et al., Cell. 2014; 156(4):633-48.
35. Mascola and Haynes. Immunological reviews. 2013; 254(1):225-44.
36. Klein et al., Science. 2013; 341(6151):1199-204.
37. Burton et al., Annual review of immunology. 2016; 34:635-59.
38. Klein et al., Cell. 2013; 153(1):126-38.
39. Xiao et al., Biochem Biophys Res Commun. 2009; 390(3):404-9.
40. Sok et al., PLoS pathogens. 2013; 9(11):e1003754
41. Bonsignori et al., Cell. 2016; 165(2):449-63.
42. Stettler et al., Science. 2016; 353(6301):823-6.
43. Priyamvada et al., PNAS USA 2016; 113(28):7852-7.
44. Dejnirattisai et al., Nature immunology. 2015; 16(2):170-7.
45. Wang et al., Science translational medicine. 2016; 8(369):369ra179
46. Tiller et al., Journal of immunological methods. 2008; 329(1-2):112-24.
47. Ye et al., Nucleic acids research. 2013; 41(Web Server issue):W34-40.
48. Giudicelli et al., IMGT/V-QUEST: IMGT standardized analysis of the immunoglobulin (IG) and T cell receptor (TR) nucleotide sequences. Cold Spring Harb Protoc. 2011; 2011(6):695-715.
49. de Alwis R, de Silva A M. Methods in molecular biology. 2014; 1138:27-39.
50. Kraus et al., Journal of clinical microbiology. 2007; 45(11):3777-80.
51. Durbin et al., The American journal of tropical medicine and hygiene. 2001; 65(5):405-13.
52. Lefranc et al., Dev Comp Immunol. 2003; 27(1):55-77.
53. Kalinke et al., Immunity. 1996; 5(6):639-52.
54. Thornburg et al., The Journal of clinical investigation. 2016; 126(4):1482-94.
55. Kato et al., Journal of virology. 2015; 89(2):1468-73.
56. Godoy-Lozano et al., Genome Med. 2016; 8(1):23 doi
57. Kalinke et al., PNAS USA 2000; 97(18):10126-31.
58. Takemori et al., European journal of immunology. 2014; 44(5):1258-64.
59. Roost et al., PNAS USA. 1995; 92(5):1257-61.
60. Hangartner et al., Nature reviews Immunology. 2006; 6(3):231-43.
61. Wakerley et al., Nat Rev Neurol. 2014; 10(9):537-44.
62. Kaveri et al., Clin Exp Immunol. 1991; 86(2):192-8.
63. Wrammert et al., Journal of virology. 2012; 86(6):2911-8.
64. Fink K., Frontiers in immunology. 2012; 3:78
65. Sukupolvi-Petty et al., Journal of virology. 2010; 84(18):9227-39.
66. Davidson et al., Immunology. 2014; 143(1):13-20.
67. Sapparapu G, et al., Nature. 2016; 540(7633):443-7.
68. Rabe et al., MMWR Morb Mortal Wkly Rep. 2016; 65(21):543-6.
69. Fields B N, Knipe D M, Howley P M. Fields virology. 5th ed. Philadelphia: Wolters Kluwer Health/Lippincott Williams & Wilkins; 2007.
70. Barba-Spaeth et al., Nature. 2016. doi: 10.1038/nature18938.
71. Stettler et al., Science. 2016. doi: 10.1126/science.aaf8505
72. Huzly et al., Euro Surveill. 2016; 21(16):30203. https://doi.org/10.2807/1560
73. Speer S D, Pierson T C. VIROLOGY. Diagnostics for Zika virus on the horizon. Science. 2016; 353(6301):750-1.

The following is a list of references cited throughout Example 10:
1. Dick G W. Zika virus. II. Trans R Soc Trop Med Hyg. 1952; 46(5):521-34.
2. Dick et al., Trans R Soc Trop Med Hyg. 1952; 46(5):509-20.
3. Speer S D, Pierson T C. Science. 2016; 353(6301):750-1. doi: 10.1126/science.aah6187.
4. Fields B N, Knipe D M. Fields virology. 2nd ed. New York: Raven Press; 1990.
5. Brasil et al., N Engl J Med. 2016; 375(24):2321-34. doi: 10.1056/NEJMoa1602412.
6. Musso et al., Clin Microbiol Infect. 2014; 20(10):0595-6. doi: 10.1111/1469-0691.12707.
7. Food and Drug Administration. Zika Virus Emergency Use Authorization [cited 2017 Aug. 21]. Available from: https://www.fda.gov/MedicalDevices/Safety/EmergencySituations/ucm161496. htm-zika.
8. Paz-Bailey et al., N Engl J Med. 2017. doi: 10.1056/NEJMoa1613108. PubMed PMID: 28195756.
9. Granger et al., J Clin Microbiol. 2017; 55(7):2127-36.
10. Magnani et al., PLoS Negl Trop Dis. 2017; 11(6):e0005655. Epub 2017/06/13. doi: 10.1371/journal.pntd.0005655.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. An assay system comprising a porous matrix comprising at least three zones, Zone A, Zone B, and Zone C, wherein
Zone A comprises an antibody or antigen-binding fragment thereof that binds to a Zika virus (ZIKV) protein and does not bind to a Dengue virus (DENV) protein, wherein the antibody or antigen binding fragment thereof is not bound to a Zika virus,
Zone B comprises an antibody or antigen-binding fragment thereof that binds to a ZIKV protein and does not bind to a DENV protein, wherein the antibody or antigen binding fragment thereof is bound to a Zika virus, and
Zone C comprises a secondary antibody which binds the antibody or antigen-binding fragment thereof of Zone A and Zone B, optionally, wherein the secondary antibody binds to the Fc of the antibody of Zone A and Zone B.

2. The assay system of claim 1, wherein the porous matrix comprises nitrocellulose of polyvinylidene fluoride (PVDF).

3. The assay system of claim 1 or 2, wherein Zones A to C are arranged along a horizontal axis, wherein each of Zones A to C is flanked by an intervening zone of the porous matrix lacking the antibody or antigen-binding fragment thereof.

4. The assay system of any one of claims 1 to 3, further comprising a sample application pad, a particle conjugate zone, a wick, and/or a backing.

5. The assay system of claim 4, wherein the porous matrix, the sample application pad, the particle conjugate zone, and the wick are arranged along a horizontal axis, optionally, wherein the horizontal axis is the same as the horizontal axis along which Zones A to C are arranged.

6. The assay system of claim 5, arranged such that the sample application pad and the wick are located at opposite ends of the assay system along the horizontal axis, wherein the particle conjugate zone is flanked by the sample application pad and the porous matrix and the porous matrix is flanked by the particle conjugate and the wick.

7. The assay system of any one of claims 4 to 6, wherein the backing is positioned below the porous matrix, the sample application pad, the particle conjugate zone, and the wick.

8. The assay system of any one of claims 4 to 7, wherein the particle conjugate zone is bound to a conjugate comprising an antibody or antigen-binding fragment thereof that binds to a Zika virus (ZIKV) protein and does not bind to a Dengue virus (DENV) protein, bound to an element or polymer.

9. The assay system of claim 8, wherein the element is a gold particle or the polymer is polystyrene.

10. The assay system of claim 8 or 9, wherein the conjugate comprises an antibody comprising the amino acid sequences of any one or more of SEQ ID NOs: 1-6, optionally, comprising the amino acid sequences of any one or more of SEQ ID NOs: 11-16.

11. The assay system of claim 10, wherein the conjugate comprises an antibody comprising the amino acid sequence of SEQ ID NO: 9 or 10 or comprising both SEQ ID NOs: 9 and 10.

12. The assay system of any one of claims 4 to 11, wherein the sample application pad comprises cellulose or glass fiber.

13. The assay system of any one of claims 4 to 12, wherein the wick comprises nitrocellulose.

14. The assay system of any one of claims 4 to 13, wherein each of Zone A and Zone B is bound to an antibody according to any one of claims 17-19.

15. The assay system of any one of claims 8 to 14, wherein the antibody bound to each of Zone A and Zone B has an Fc which is the same as the Fc of the antibody of the conjugate bound to the particle conjugate zone.

16. The assay system of any one of claims 1-15, wherein the antibody or antigen-binding fragment of claim 1, which does not bind to a protein of any one of DENV subtype 1, DENV subtype 2, DENV subtype 3, and DENV subtype 4.

17. The assay system of claim 16, which does not bind to any flavivirus other than ZIKV.

18. The assay system of any one of the previous claims, wherein the ZIKV protein is from a ZIKV comprising the genome of GenBank Accession No. KU926309.1 (SEQ ID NO: 54).

19. The assay system of any one of claims 1-9, comprising the amino acid sequences of any one or more of SEQ ID NOs: 21-26, 29-34, 37-42, 45-50, 58-63, or 106-111.

20. The assay system of any one of claims 1-9, comprising the amino acid sequence of SEQ ID NO: 27, 28, 35, 36, 43, 44, 51, 52, 64, 65, 67, or 69.

21. The assay system of any one of claims 1-9, comprising the amino acid sequence of SEQ ID NO: 27 and 28, SEQ ID NOs: 35 and 36, SEQ ID NOs: 43 and 44, SEQ ID NOs: 51 and 52, SEQ ID NOs: 64 and 65, or SEQ ID NOs: 67 and 69.

22. The assay system of any one of the previous claims, comprising a non-human heavy chain constant region and/or a non-human light chain constant region.

23. The assay system of claim 22, comprising a mouse, goat, rabbit, or non-human primate heavy chain constant region and/or a mouse, goat, rabbit, or non-human primate non-human light chain constant region.

24. The assay system of claim 23, comprising a heavy chain constant region and a light chain constant region of the Cercopithecidae family of primates.

25. The assay system of claim 24, comprising a heavy chain constant region and a light chain constant region of Rhesus monkey.

26. The assay system of any one of the previous claims, wherein the heavy chain constant region comprises the amino acid sequence of SEQ ID NO: 19 and/or 20.

27. A method of detecting a Zika virus (ZIKV) infection and ZIKV immunity in a subject, comprising adding a sample obtained from a subject to the assay system according to any one of claims 1-26, wherein, when the assay system exhibits a single band in Zone C, the subject is determined as having neither a ZIKV infection nor ZIKV immunity, when the assay system exhibits a band in each of Zone A and Zone B, the subject is determined as having both a ZIKV infection and ZIKV immunity, and when the assay system exhibits a band in Zone B and a band is absent in Zone A, the subject is determined as not having a ZIKV infection but having ZIKV immunity.

28. The method of claim 27, wherein the sample is blood, plasma, serum, or urine.

29. The method of claim 28, wherein the sample is blood or urine.

30. The method of any one of claims 27 to 29, wherein the method of detecting Zika virus (ZIKV) immunity or ZIKV exposure detects the presence or absence of ZIKV-specific antibodies made by the subject.

31. The method of any one of claims 27 to 30, wherein the subject is a mammal.

32. The method of claim 31, wherein the mammal is a human.

33. The method of claim 32, wherein the human is female.

34. The method of claim 33, wherein the female human is pregnant or is considering whether or not to become pregnant.

35. A method of assessing efficacy of a Zika virus (ZIKV) vaccine in a subject who has received a ZIKV vaccine, comprising (i) adding a sample obtained from the subject to the assay system according to any one of claims 1-26, wherein, when the assay system exhibits (A) a band in each of Zone A and Zone B or (B) a band in Zone B and a band is absent in Zone A, the ZIKV vaccine is determined as effective in the subject, and when when the assay system exhibits a single band in Zone C, the ZIKV vaccine is determined as ineffective in the subject.


SEQUENCE LISTING (111 sequences)

SEQ ID NO: 1 - protein, 8 aa, Artificial Sequence
  Synthetic peptide
  MISC_FEATURE: CLONE CODE 17 HCDR1
GFTFSSYW

SEQ ID NO: 2 - protein, 8 aa, Artificial Sequence
  Synthetic peptide
  MISC_FEATURE: CLONE CODE 17 HCDR2
IKQDGSEK

SEQ ID NO: 3 - protein, 22 aa, Artificial Sequence
  Synthetic peptide
  MISC_FEATURE: CLONE CODE 17 HCDR3
AGNGWDDSSGYYYRNYYYGMDV

SEQ ID NO: 4 - protein, 6 aa, Artificial Sequence
  Synthetic peptide
  MISC_FEATURE: CLONE CODE 17 LCDR4
QGISSY

SEQ ID NO: 5 - protein, 3 aa, Artificial Sequence
  Synthetic peptide
  MISC_FEATURE: CLONE CODE 17 LCDR5
AAS

SEQ ID NO: 6 - protein, 9 aa, Artificial Sequence
  Synthetic peptide
  MISC_FEATURE: CLONE CODE 17 LCDR6
QQYYSYPRT

SEQ ID NO: 7 - DNA, 387 bp, Artificial Sequence
  Synthetic polynucleotide
  misc_feature: CLONE CODE 17 heavy variable
caggtgcagctggtgcagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcggggaatggatgggacgacagtagtggttattactaccggaactactactacggtatggacgtctggggccaagggaccacggtcaccgtctcctca

SEQ ID NO: 8 - DNA, 321 bp, Artificial Sequence
  Synthetic polynucleotide
  misc_feature: CLONE CODE 17 light variable
gacatcgtgatgacccagtctccatcctcattctctgcatctacaggagacagagtcaccatcacttgtcgggcgagtcagggtattagcagttatttagcctggtatcagcaaaaaccagggaaagcccctaagctcctgatctatgctgcatccactttgcaaagtggggtcccatcaaggttcagcggcagtggatctgggacagatttcactctcaccatcagctgcctgcagtctgaagattttgcaacttattactgtcaacagtattatagttaccctcgaactttcggcggagggaccaaagtggatatcaaa

SEQ ID NO: 9 - protein, 129 aa, Artificial Sequence
  Synthetic peptide
  MISC_FEATURE: CLONE CODE 17 heavy variable
QVQLVQSGGGLVQPGGSLRLSCAASGFTFSSYWMSWVRQAPGKGLEWVANIKQDGSEKYYVDSVKGRFTISRDNAKNSLYLQMNSLRAEDTAVYYCAGNGWDDSSGYYYRNYYYGMDVWGQGTTVTVSS

SEQ ID NO: 10 - protein, 107 aa, Artificial Sequence
  Synthetic peptide
  MISC_FEATURE: CLONE CODE 17 light variable
DIQMTQSPSSFSASTGDRVTITCRASQGISSYLAWYQQKPGKAPKLLIYAASTLQSGVPSRFSGSGSGTDFTLTISCLQSEDFATYYCQQYYSYPRTFGGGTKVDIK

SEQ ID NO: 11 - protein, 24 aa, Artificial Sequence
  Synthetic peptide
  MISC_FEATURE: CLONE CODE 17 FR1
VQLVQSGGGLVQPGGSLRLSCAAS

SEQ ID NO: 12 - protein, 17 aa, Artificial Sequence
  Synthetic peptide
  MISC_FEATURE: CLONE CODE 17 FR2
MSWVRQAPGKGLEWVAN

SEQ ID NO: 13 - protein, 38 aa, Artificial Sequence
  Synthetic peptide
  MISC_FEATURE: CLONE CODE 17 FR3
YYVDSVKGRFTISRDNAKNSLYLQMNSLRAEDTAVYYC

SEQ ID NO: 14 - protein, 25 aa, Artificial Sequence
  Synthetic peptide
  MISC_FEATURE: CLONE CODE 17 FR4
IVMTQSPSSFSASTGDRVTITCRAS

SEQ ID NO: 15 - protein, 17 aa, Artificial Sequence
  Synthetic peptide
  MISC_FEATURE: CLONE CODE 17 FR5
LAWYQQKPGKAPKLLIY

SEQ ID NO: 16 - protein, 36 aa, Artificial Sequence
  Synthetic peptide
  MISC_FEATURE: CLONE CODE 17 FR6
TLQSGVPSRFSGSGSGTDFTLTISCLQSEDFATYYC

SEQ ID NO: 17 - protein, 672 aa, Zika virus
  ref <308>: DATABASE ACCESSION NUMBER: GenBank / KU926309.1
  ref <309>: DATABASE ENTRY DATE: 2016-03-21
  ref <313>: RELEVANT RESIDUES IN SEQ ID NO: (1)..(672)
AEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 18 - protein, 234 aa, Zika virus
  ref <308>: DATABASE ACCESSION NUMBER: GenBank / KU926309.1
  ref <309>: DATABASE ENTRY DATE: 2016-03-21
  ref <313>: RELEVANT RESIDUES IN SEQ ID NO: (90)..(323)
RSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVND

SEQ ID NO: 19 - protein, 333 aa, Artificial Sequence
  Synthetic peptide
  MISC_FEATURE: Rhesus heavy chain constant
ASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCPAPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSVSPGK

SEQ ID NO: 20 - protein, 107 aa, Artificial Sequence
  Synthetic peptide
  MISC_FEATURE: Rhesus light chain constant
RAVAAPSVFIFPPSEDQVKSGTVSVVCLLNNFYPREASVKWKVDGVLKTGNSQESVTEQDSKDNTYSLSSTLTLSSTDYQSHNVYACEVTHQGLSSPVTKSFNRGEC

SEQ ID NO: 21 - protein, 8 aa, Artificial Sequence
  Synthetic peptide
  MISC_FEATURE: CLONE CODE 27 HCDR1
GYTFTSYD

SEQ ID NO: 22 - protein, 8 aa, Artificial Sequence
  Synthetic peptide
  MISC_FEATURE: CLONE CODE 27 HCDR2
MNPNSGNT

SEQ ID NO: 23 - protein, 21 aa, Artificial Sequence
  Synthetic peptide
  MISC_FEATURE: CLONE CODE 27 HCDR3
ARGGLYDFWSGYHYYYYGMDV

SEQ ID NO: 24 - protein, 6 aa, Artificial Sequence
  Synthetic peptide
  MISC_FEATURE: CLONE CODE 27 HCDR4
QSVSSN

SEQ ID NO: 25 - protein, 3 aa, Artificial Sequence
  Synthetic peptide
  MISC_FEATURE: CLONE CODE 27 HCDR5
GAS

SEQ ID NO: 26 - protein, 10 aa, Artificial Sequence [sequence omitted]

SEQ ID NO: 27 - protein, 128 aa, Artificial Sequence
  Synthetic peptide
  MISC_FEATURE: CLONE CODE 27 heavy variable
QVQLVESGAEVKKPGASVKVSCKASGYTFTSYDINWVRQATGQGLEWMGRMNPNSGNTGYAQKFQGRVTMTRNTSISTAYMELSSLRSEDTAVYYCARGGLYDFWSGYHYYYYGMDVWGQGTTVTVSS

SEQ ID NO: 28 - protein, 108 aa, Artificial Sequence
  Synthetic peptide
  MISC_FEATURE: CLONE CODE 27 light variable
EIVMTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVDIK

SEQ ID NO: 29 - protein, 8 aa, Artificial Sequence
  Synthetic peptide
  MISC_FEATURE: CLONE CODE 21 HCDR1
GFTFSSYW

SEQ ID NO: 30 - protein, 8 aa, Artificial Sequence
  Synthetic peptide
  MISC_FEATURE: CLONE CODE 21 HCDR2
INSDGSST

SEQ ID NO: 31 - protein, 24 aa, Artificial Sequence
  Synthetic peptide
  MISC_FEATURE: CLONE CODE 21 HCDR3
ARDLRPAYYYDSSGYYRTVYGMDV

SEQ ID NO: 32 - protein, 9 aa, Artificial Sequence
  Synthetic peptide
  MISC_FEATURE: CLONE CODE 21 HCDR4
SSDVGGYNY

SEQ ID NO: 33 - protein, 3 aa, Artificial Sequence
  Synthetic peptide
  MISC_FEATURE: CLONE CODE 21 HCDR5
DVS

SEQ ID NO: 34 - protein, 10 aa, Artificial Sequence
  Synthetic peptide
  MISC_FEATURE: CLONE CODE 21 HCDR6
SSYTSSSTLV

SEQ ID NO: 35 - protein, 131 aa, Artificial Sequence
  Synthetic peptide
  MISC_FEATURE: CLONE CODE 21 heavy variable
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARDLRPAYYYDSSGYYRTVYGMDVWGQGTTVTVSS

SEQ ID NO: 36 - protein, 110 aa, Artificial Sequence
  Synthetic peptide
  MISC_FEATURE: CLONE CODE 21 light variable
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLVFGGGTKLTVL

SEQ ID NO: 37 - protein, 8 aa, Artificial Sequence
  Synthetic peptide
  MISC_FEATURE: CLONE CODE 28 HCDR1
GGSFGGYY

SEQ ID NO: 38 - protein, 7 aa, Artificial Sequence
  Synthetic peptide
  MISC_FEATURE: CLONE CODE 28 HCDR2
IDSSGSA

SEQ ID NO: 39 - protein, 23 aa, Artificial Sequence
  Synthetic peptide
  MISC_FEATURE: CLONE CODE 28 HCDR3
ARGLLNDTDSGGYYRGGFYYFDY

SEQ ID NO: 40 - protein, 12 aa, Artificial Sequence
  Synthetic peptide
  MISC_FEATURE: CLONE CODE 28 LCDR4
QSVLYSSNNKNY

SEQ ID NO: 41 - protein, 3 aa, Artificial Sequence
  Synthetic peptide
  MISC_FEATURE: CLONE CODE 28 LCDR5
WAS

SEQ ID NO: 42 - protein, 9 aa, Artificial Sequence
  Synthetic peptide
  MISC_FEATURE: CLONE CODE 28 LCDR6
QQYSSFPPS

SEQ ID NO: 43 - protein, 129 aa, Artificial Sequence
  Synthetic peptide
  MISC_FEATURE: CLONE CODE 28 heavy variable
QLQLQEWGAGLLKPSETLSLTCAVYGGSFGGYYWTWIRQSPGKGLEWIGKIDSSGSANYNPSLKSRLTISVESSKNQFSLELNSVTAADTAVYYCARGLLNDTDSGGYYRGGFYYFDYWGQGTLVTVSS

SEQ ID NO: 44 - protein, 113 aa, Artificial Sequence
  Synthetic peptide
  MISC_FEATURE: CLONE CODE 28 light variable
DIVMTQSPDSLAVSLGERATINCKSSQSVLYSSNNKNYLAWYQQKPGQPPKLLIYWASTRESGVPARFSGRGSGTDFTLTISSLQAEDVAVYFCQQYSSFPPSFGQGTKVEIK

SEQ ID NO: 45 - protein, 8 aa, Artificial Sequence
  Synthetic peptide
  MISC_FEATURE: CLONE CODE 29 HCDR1
GGSISSYY

SEQ ID NO: 46 - protein, 7 aa, Artificial Sequence
  Synthetic peptide
  MISC_FEATURE: CLONE CODE 29 HCDR2
IYYSGST

SEQ ID NO: 47 - protein, 15 aa, Artificial Sequence
  Synthetic peptide
  MISC_FEATURE: CLONE CODE 29 HCDR3
ARHGGVPSYYYGMDV

SEQ ID NO: 48 - protein, 9 aa, Artificial Sequence
  Synthetic peptide
  MISC_FEATURE: CLONE CODE 29 LCDR4
SSNIGAGYD

SEQ ID NO: 49 - protein, 3 aa, Artificial Sequence
  Synthetic peptide
  MISC_FEATURE: CLONE CODE 29 LCDR5
GNS

SEQ ID NO: 50 - protein, 13 aa, Artificial Sequence
  Synthetic peptide
  MISC_FEATURE: CLONE CODE 29 LCDR6
QSYDSSLSGSNWV

SEQ ID NO: 51 - protein, 121 aa, Artificial Sequence
  Synthetic peptide
  MISC_FEATURE: CLONE CODE 29 heavy variable
EVQLVESGPGLVKPSETLSLTCTVSGGSISSYYWSWIRQPPGKGLEWIGYIYYSGSTNYNPSLKSRVTISVDTSKNQFSLKLSSVTAADTAVYYCARHGGVPSYYYGMDVWGQGTTVTVSS

SEQ ID NO: 52 - protein, 113 aa, Artificial Sequence
  Synthetic peptide
  MISC_FEATURE: CLONE CODE 29 light variable
QPVLTQPPSVSGAPGQRVTISCTGSSSNIGAGYDVHWYQQLPGTAPKLLIYGNSNRPSGVPDRFSGSKSGTSASLAISGLQAEDEADYYCQSYDSSLSGSNWVFGGGTKLTVL

SEQ ID NO: 53 - protein, 3423 aa, Zika virus
  ref <308>: DATABASE ACCESSION NUMBER: GenBank / AMQ48981.1
  ref <309>: DATABASE ENTRY DATE: 2016-03-21
  ref <313>: RELEVANT RESIDUES IN SEQ ID NO: (1)..(3423)
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPIAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMRRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 54 - DNA, 10795 bp, Zika virus
  ref <308>: DATABASE ACCESSION NUMBER: GenBank / KU926309.1
  ref <309>: DATABASE ENTRY DATE: 2016-03-21
  ref <313>: RELEVANT RESIDUES IN SEQ ID NO: (1)..(10795)
gatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactacatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcgagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggataaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattgccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggcctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatcggtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatagccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccttttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctgggttcaacacaagttggagtgggagttatgcaagagggggtctttcacactatgtggcacgtcacaaaaggatccgcactgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgagaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgttgggaacagtctcgctgggaatctttttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctacggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgcagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgacaggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccatagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatg

SEQ ID NO: 55 - protein, 168 aa, Artificial Sequence
  Synthetic peptide
  ref <308>: DATABASE ACCESSION NUMBER: GenBank / KU926309.1
  ref <309>: DATABASE ENTRY DATE: 2016-03-21
  ref <313>: RELEVANT RESIDUES IN SEQ ID NO: (1)..(168)
AEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYS

SEQ ID NO: 56 - protein, 75 aa, Artificial Sequence
  Synthetic peptide
  ref <308>: DATABASE ACCESSION NUMBER: GenBank / KU926309.1
  ref <309>: DATABASE ENTRY DATE: 2016-03-21
  ref <313>: RELEVANT RESIDUES IN SEQ ID NO: (94)..(168)
AVTLPSHSTRKLQTRSQTWLESREYTKHLIRVENWIFRNPGFALAAAAIAWLLGSSTSQKVIYLVMILLIAPAYS

SEQ ID NO: 57 - protein, 504 aa, Artificial Sequence
  Synthetic peptide
  ref <308>: DATABASE ACCESSION NUMBER: GenBank / KU926309.1
  ref <309>: DATABASE ENTRY DATE: 2016-03-21
  ref <313>: RELEVANT RESIDUES IN SEQ ID NO: (169)..(672)
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 58 - protein, 8 aa, Artificial Sequence
  Synthetic peptide
  MISC_FEATURE: CC4 HCDR1
GGSISSYY

SEQ ID NO: 59 - protein, 7 aa, Artificial Sequence
  Synthetic peptide
  MISC_FEATURE: CC4 HCDR2
IYTSGST

SEQ ID NO: 60 - protein, 11 aa, Artificial Sequence
  Synthetic peptide
  MISC_FEATURE: CC4 HCDR3
ARESGSLYMDV

SEQ ID NO: 61 - protein, 6 aa, Artificial Sequence
  Synthetic peptide
  MISC_FEATURE: CLONE CODE LCDR4
QSVSSY

SEQ ID NO: 62 - protein, 3 aa, Artificial Sequence
  Synthetic peptide
  MISC_FEATURE: CC4 LCDR5
DAS

SEQ ID NO: 63 - protein, 9 aa, Artificial Sequence
  Synthetic peptide
  MISC_FEATURE: CC4 LCDR6
QQRSNWPLT

SEQ ID NO: 64 - protein, 118 aa, Artificial Sequence
  Synthetic peptide
  MISC_FEATURE: CC4 variable heavy
QVQLVQESGPGPVKPSETLSLTCTVSGGSISSYYWSWIRQPAGKGLEWIGRIYTSGSTNYNPSLKSRVTMSVDTSKNQFSLKLSSVTAADTAVYYCARESGSLYMDVWGKGTAVTVSS

SEQ ID NO: 65 - protein, 107 aa, Artificial Sequence
  Synthetic peptide
  MISC_FEATURE: CC4 variable light
DVVMTQSPATLSLPPGERATLSCRASQSVSSYLAWYQQKPGQAPRLLIYDASNRATGIPARFSGSGSGTDFTLTISSLEPEDFAVYYCQQRSNWPLTFGGGTKVDIK

SEQ ID NO: 66 - DNA, 375 bp, Artificial Sequence
  Synthetic Polynucleotide
  misc_feature: 1306D13_P3E11
caggtgcagctggtggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgcccctcaagagtcgagttaccatatcagtagacacgtctaagaaccagttctccctgaagctgagctctgtgactgccgcggacacggccgtgtattactgtgcgagagccccccccgtttgggggagttatcgtccctactactttgactactggggccagggaaccctggtcaccgtctcctca

SEQ ID NO: 67 - protein, 125 aa, Artificial Sequence
  Synthetic Polypeptide
  MISC_FEATURE: 1306D13_P3E11
QVQLVESGPGLVKPSQTLSLTCTVSGGSISSGGYYWSWIRQHPGKGLEWIGYIYYSGSTYYNPPLKSRVTISVDTSKNQFSLKLSSVTAADTAVYYCARAPPVWGSYRPYYFDYWGQGTLVTVSS

SEQ ID NO: 68 - DNA, 324 bp, Artificial Sequence
  Synthetic polynucleotide
  misc_feature: 1306D13_P3E11
gacatcgtgatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgtggacgttcggccaagggaccaaggtggaaatcaaa

SEQ ID NO: 69 - protein, 108 aa, Artificial Sequence
  Synthetic Polypeptide
  MISC_FEATURE: 1306D13_P3E11
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPWTFGQGTKVEIK

SEQ ID NO: 70 - protein, 18 aa, Artificial Sequence
  Synthetic Polypeptide
  MISC_FEATURE: IGHV5-51*01
CARHQPQGDTASHGMDVW

SEQ ID NO: 71 - protein, 11 aa, Artificial Sequence
  Synthetic Polypeptide
  MISC_FEATURE: IGKV1-39*01
CQQSFNRLWTF

SEQ ID NO: 72 - protein, 22 aa, Artificial Sequence
  Synthetic Polypeptide
  MISC_FEATURE: IGHV1-18*01
CTKIDLHWDGVNGYDVSYFENW

SEQ ID NO: 73 - protein, 11 aa, Artificial Sequence [sequence omitted]

SEQ ID NO: 74 - protein, 13 aa, Artificial Sequence
  Synthetic Polypeptide
  MISC_FEATURE: IGHV4-4*07
CARESGSLYMDVW

SEQ ID NO: 75 - protein, 11 aa, Artificial Sequence [sequence omitted]

SEQ ID NO: 76 - protein, 20 aa, Artificial Sequence
  Synthetic Polypeptide
  MISC_FEATURE: IGHV3-23*04
CAKGMYYDFWSGSNDAFDIW

SEQ ID NO: 77 - protein, 11 aa, Artificial Sequence
  Synthetic Polypeptide
  MISC_FEATURE: IGKV1-39*01
CQQSYSTPRTF

SEQ ID NO: 78 - protein, 15 aa, Artificial Sequence
  Synthetic Polypeptide
  MISC_FEATURE: IGHV4-39*07
CARTGSRRWYGMDVW

SEQ ID NO: 79 - protein, 11 aa, Artificial Sequence
  Synthetic Polypeptide
  MISC_FEATURE: IGKV3-20*01
CQQYGSSVWAF

SEQ ID NO: 80 - protein, 20 aa, Artificial Sequence
  Synthetic Polypeptide
  MISC_FEATURE: IGHV3-23*04
CAKSFYRDFWSGSNDAFDIW

SEQ ID NO: 81 - protein, 11 aa, Artificial Sequence
  Synthetic Polypeptide
  MISC_FEATURE: IGKV1-39*01
CQQSYSTPRTF

SEQ ID NO: 82 - protein, 25 aa, Artificial Sequence
  Synthetic Polypeptide
  MISC_FEATURE: IGHV4-34*01
CARRGGRCSSTSCYPYYYYYYMDVW

SEQ ID NO: 83 - protein, 13 aa, Artificial Sequence [sequence omitted]

SEQ ID NO: 84 - protein, 20 aa, Artificial Sequence
  Synthetic Polypeptide
  MISC_FEATURE: IGHV4-4*07
CAKGGVTPGGGTSGTWFNPW

SEQ ID NO: 85 - protein, 12 aa, Artificial Sequence
  Synthetic Polypeptide
  MISC_FEATURE: IGKV3-15*01
CQQYDHWPPYTF

SEQ ID NO: 86 - protein, 20 aa, Artificial Sequence
  Synthetic Polypeptide
  MISC_FEATURE: IGHV3-23*04
CAKSFYRDFWSGSNDAFDIW

SEQ ID NO: 87 - protein, 11 aa, Artificial Sequence
  Synthetic Polypeptide
  MISC_FEATURE: IGKV1-39*01
CQQSYSTPRTF

SEQ ID NO: 88 - protein, 24 aa, Artificial Sequence
  Synthetic Polypeptide
  MISC_FEATURE: IGHV3-7*01
CAGNGWDDSSGYYYRNYYYGMDVW

SEQ ID NO: 89 - protein, 11 aa, Artificial Sequence [sequence omitted]

SEQ ID NO: 90 - protein, 16 aa, Artificial Sequence
  Synthetic Polypeptide
  MISC_FEATURE: IGHV4-59*08
CVRHEIAGTTGAFDIW

SEQ ID NO: 91 - protein, 11 aa, Artificial Sequence [sequence omitted]

SEQ ID NO: 92 - protein, 15 aa, Artificial Sequence
  Synthetic Polypeptide
  MISC_FEATURE: IGHV3-21*01
CARGRYGYSYALDYW

SEQ ID NO: 93 - protein, 12 aa, Artificial Sequence [sequence omitted]

SEQ ID NO: 94 - protein, 12 aa, Artificial Sequence
  Synthetic Polypeptide
  MISC_FEATURE: IGHV4-39*07
CARKAGYYYDYW

SEQ ID NO: 95 - protein, 10 aa, Artificial Sequence
  Synthetic Polypeptide
  MISC_FEATURE: IGLV3-1*01
CQTGDTTTVF

SEQ ID NO: 96 - protein, 19 aa, Artificial Sequence
  Synthetic Polypeptide
  MISC_FEATURE: IGHV3-21*01
CARDFRGGYYGSGDAFDIW

SEQ ID NO: 97 - protein, 14 aa, Artificial Sequence [sequence omitted]

SEQ ID NO: 98 - protein, 17 aa, Artificial Sequence
  Synthetic Polypeptide
  MISC_FEATURE: GHV4-34*01
CARKRWQQLRRNWFDPW

SEQ ID NO: 99 - protein, 13 aa, Artificial Sequence [sequence omitted]

SEQ ID NO: 100 - protein, 18 aa, Artificial Sequence
  Synthetic Polypeptide
  MISC_FEATURE: IGHV3-23*04
CAKVRRVVVIVHDAFDVW

SEQ ID NO: 101 - protein, 11 aa, Artificial Sequence
  Synthetic Polypeptide
  MISC_FEATURE: IGKV1-16*02
CQQYSFYPLTF

SEQ ID NO: 102 - protein, 16 aa, Artificial Sequence
  Synthetic Polypeptide
  MISC_FEATURE: IGHV3-23*04
CAKVVGLRLDDAFDIW

SEQ ID NO: 103 - protein, 10 aa, Artificial Sequence
  Synthetic Polypeptide
  MISC_FEATURE: IGKV1-5*03
CQQYLSYCTF

SEQ ID NO: 104 - protein, 22 aa, Artificial Sequence
  Synthetic Polypeptide
  MISC_FEATURE: IGHV3-23*04
CAKDRGYYDSSGYYYSEAFDYW

SEQ ID NO: 105 - protein, 12 aa, Artificial Sequence
  Synthetic Polypeptide
  MISC_FEATURE: IGKV3-11*01
CQQRSNWSGITF

SEQ ID NO: 106 - protein, 10 aa, Artificial Sequence
  Synthetic Polypeptide
  MISC_FEATURE: P3E11 Heavy CDR-1
GGSISSGGYY

SEQ ID NO: 107 - protein, 7 aa, Artificial Sequence
  Synthetic Polypeptide
  MISC_FEATURE: P3E11 Heavy CDR-2
IYYSGST

SEQ ID NO: 108 - protein, 17 aa, Artificial Sequence
  Synthetic Polypeptide
  MISC_FEATURE: P3E11 Heavy CDR-3
ARAPPVWGSYRPYYFDY

SEQ ID NO: 109 - protein, 17 aa, Artificial Sequence
  Synthetic Polypeptide
  MISC_FEATURE: P3E11 Light CDR-1
ARAPPVWGSYRPYYFDY

SEQ ID NO: 110 - protein, 3 aa, Artificial Sequence
  Synthetic Polypeptide
  MISC_FEATURE: P3E11 Light CDR-2
AAS

SEQ ID NO: 111 - protein, 10 aa, Artificial Sequence
  Synthetc Polypeptide
  MISC_FEATURE: P3E11 Light CDR-3
QQSYSTPPWT